(19) United States
(12) Patent Application Publication
(10) Pub. No.: US 2025/0327101 A1
(43) Pub. Date: Oct. 23, 2025

(54) BIO-RECYCLING OF POLYESTERS INTO PHA

(71) Applicant: Société des Produits Nestlé S.A., Vevey (CH)

(72) Inventors: Rosa Maria Monica Aragão Peralta Börner, Epalinges (CH); Tim Börner, Epalinges (CH); Raúl Muñoz Torre, Valladolid (ES); Fernando Santos Beneit, Valladolid (ES); Sergio Bordel Velasco, Valladolid (ES)

(73) Assignee: Société des Produits Nestlé S.A., Vevey (CH)

(21) Appl. No.: 19/121,107

(22) PCT Filed: Oct. 18, 2023
(86) PCT No.: PCT/EP2023/078940

§ 371 Date: Apr. 14, 2025

(30) Foreign Application Priority Data
Oct. 20, 2022 (EP) 22202651.0

(51) Int. Cl. C12P 7/625; C08J 11/04; C12R 1/01

(52) U.S. Cl. CPC C12P 7/625; C08J 11/04; C08J 2333/02; C08J 2367/02; C12R 2001/01

(57) ABSTRACT

A method for producing polyhydroxyalkanoate (PHA) from polyester waste is provided. The method includes the steps of: (a) providing a culture broth comprising polyester waste; and (b) cultivating a microbe in the culture broth to produce PHA. The method may utilize a wide variety of monomers and may proceed via a one-step process.

### SEQUENCE LISTING

This application contains a sequence listing in computer readable form (file name: 19192-010001_SequenceListing.xml; date of creation: Mar. 5, 2025; file size: 90 kb) which is incorporated herein by reference in its entirety and forms part of the disclosure.

### FIELD OF THE INVENTION

The present invention concerns a method for producing polyhydroxyalkanoate (PHA) from polyester waste. The present invention also concerns PHA produced by said method and articles made using said PHA.

### BACKGROUND OF THE INVENTION

Plastics enjoy widespread use due to their adaptability, light weight, durability, and flexibility. However, none of the commonly used plastics are biodegradable. As a result, they accumulate, rather than decompose, in landfills or the natural environment. As of 2015, approximately 6300 Mt of plastic waste had been generated, only around 9% of which had been recycled, 12% was incinerated, and 79% was accumulated in landfills or the natural environment (see Geyer, R., et al., 2017. Science advances, 3 (7), p.e1700782).

The degradation of plastics generally occurs slowly in nature and involves various environmental factors such as temperature, moisture, pressure and action of microorganisms. To accelerate degradation, plastic waste can be degraded with physical process such as soil burial or combustion, or chemical processes such as by photo-oxidation hydrolysis or degradation with specific and harsh chemicals. However, both physical and chemical methods are associated with significant drawbacks (see Bano, K., et al, 2017. Current pharmaceutical biotechnology, 18 (5), pp. 429-440).

An alternative to petroleum-based plastics is bio-based polyesters. Polyhydroxyalkanoates (PHAs) are structurally diverse microbial polyesters synthesized by numerous prokaryotic microorganisms. Since PHAs are biocompatible, bioresorbable, and biodegradable, they have a reduced impact on the environment. When PHA-based products are left in the environment, they are degraded into CO₂, H₂O, and CH₄, which facilitate the natural cycle of circulatory and renewability. However, the bacterial synthesis of PHA is currently not cost-effective compared to petroleum-based plastics.

Current technologies employ a two-step microbial process that converts organic waste, including PHA waste, into volatile fatty acids (VFA) under anoxic conditions and then in a second aerobic fermentation step converts the VFA into PHA (see Riaz, S., et al., 2021. Polymers, 13 (2), p.253).

Accordingly, there is a demand for new processes to degrade plastic waste and also for new processes to produce PHA.

### SUMMARY OF THE INVENTION

The present inventors have developed a direct and efficient method for producing polyhydroxyalkanoate (PHA) from polyester waste.

The present inventors have shown that the method allows the utilisation of a wide variety of polyester monomers, including polyester monomers that are difficult for biological metabolism, such as 1,4-butanediol. The method allows the utilisation of mixed polyester waste comprising biodegradable polyesters (e.g. PHA, PHB, PHBH) as well as non-biodegradable polyesters (e.g. PET).

The present inventors have shown that the method may proceed via a one-step process, carrying out cultivation using a single microorganism. This is in contrast to the two-step processes currently carried out. The present inventors have also shown that the cultivation can be carried out under aerobic or anoxic conditions. This flexibility may reduce the need for oxygen in fermentation tanks, which is usually a limitation in large scale fermentation.

The present inventors have also identified a microorganism with suitable pathways for utilising mixed polyester waste by constructing a new genomic metabolic model. The present inventors have identified the genes involved in said pathways.

In one aspect, the present invention provides a method for producing polyhydroxyalkanoate (PHA) from polyester waste, the method comprising the steps of: (a) providing a culture broth comprising polyester waste; and (b) cultivating a microbe in the culture broth to produce PHA.

The microbe may utilise one or more polyester monomer from the polyester waste to produce the PHA. The microbe may utilise a plurality of polyester monomers from the polyester waste to produce the PHA. Suitably, the microbe utilises at least three, at least four, at least five, at least six, at least seven, or at least eight polyester monomers from the polyester waste to produce the PHA. Suitably, the microbe utilises polyester monomers from a plurality of polyesters from the polyester waste to produce the PHA. Suitably, the microbe utilises polyester monomers from at least three, at least four, at least five, at least six, at least seven, or at least eight polyesters from the polyester waste to produce the PHA. In some embodiments, the microbe utilises 1,4-butanediol from the polyester waste to produce the PHA.

Any suitable microbe may be used in the method of the present invention. Suitably, the microbe is from the genus Paracoccus. Suitably, the microbe is a Paracoccus denitrificans. Suitably, the microbe is Paracoccus denitrificans DSM 413, or a derivative thereof. Suitably, the microbe is Paracoccus denitrificans DSM 413, Paracoccus denitrificans PD1222, Paracoccus denitrificans CNCM I-5881, Paracoccus denitrificans ATCC 19367, Paracoccus denitrificans ATCC 17741, Paracoccus denitrificans ATCC 13543, Paracoccus denitrificans NCIB 8944, Paracoccus denitrificans NRRL B-3785, Paracoccus denitrificans CCM 982, Paracoccus denitrificans LMD 22.21, Paracoccus denitrificans JCM 21484, Paracoccus denitrificans NBRC 102528, Paracoccus denitrificans NCCB 22021, Paracoccus denitrificans NBRC 13301, Paracoccus denitrificans NCIMB 8944, Paracoccus denitrificans DSM 15418, Paracoccus denitrificans DSM 415, Paracoccus denitrificans NCIMB 11627, Paracoccus denitrificans NCIMB 9722, Paracoccus denitrificans IMET 10380, Paracoccus denitrificans VKM B-1324, or Paracoccus denitrificans ICPB 3979.

The microbe may comprise genes encoding for two or more pathways, three or more pathways, four or more pathways, five or more pathways, six or more pathways, or seven or more pathways selected from: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol. The microbe may comprise genes encoding for each of: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol.

Any suitable polyester waste may be utilised. Suitably, the polyester waste comprises two or more, three or more, four or more, five or more, six or more, or seven or more polyester monomers selected from succinic acid, lactic acid, ethylene glycol, adipic acid, 6-hydroxycaproic acid, 3-hydroxybutyric acid, 3-hydroxyvaleric acid, and 1,4-butanediol. Suitably, the polyester waste comprises succinic acid, lactic acid, ethylene glycol, adipic acid, 6-hydroxycaproic acid, 3-hydroxybutyric acid, 3-hydroxyvaleric acid, and 1,4-butanediol. In some embodiments, the polyester waste comprises 1,4-butanediol. Suitably, the polyester waste comprises the polyester monomers in the form of free monomers. Suitably, the polyester waste comprises one or more, two or more, three or more, four or more, five or more, six or more, seven or more, or eight or more polyesters selected from: polybutylene succinate (PBS), polybutylene succinate adipate (PBSA), poly(butylene succinate-co-terephthalate) (PBST), poly(butylene succinate/terephthalate/isophthalate)-co-(lactate) (PBSTIL), polybutylene terephthalate (PBT), polybutylene adipate terephthalate (PBAT), polyethylene terephthalate (PET), poly(ethylene adipate) (PEA), polylactic acid (PLA), polycaprolactone (PCL), polyhydroxybutyrate (PHB), and poly(3-hydroxybutyrate-co-3-hydroxyvalerate) (PHBV). The polyester waste may be pre-treated. Suitably, the polyester waste is mechanically treated and/or chemically treated.

The method of the present invention may further comprise a step of pre-treating the polyester waste. Any suitable pre-treatment may be used. Suitably, the method further comprises a step of mechanically treating the polyester waste (e.g. the polyester waste may be shredded). Suitably, the method further comprises a step of chemically treating the polyester waste (e.g. the polyester waste may undergo alkaline treatment).

Any suitable culture conditions may be used. Suitably, the culture broth comprises the polyester waste in an amount of from about 1 g/L to about 100 g/L, from about 1 g/L to about 50 g/L, from about 1 g/L to about 20 g/L, from about 2 g/L to about 10 g/L, or from about 2 g/L to about 5 g/L. Suitably, the culture broth comprises mineral salt medium. Suitably, the microbe is cultivated under aerobic or anoxic conditions. In some embodiments, the microbe is cultivated under anoxic conditions. Suitably, the microbe is cultivated for from about one to about seven days, from about two to about six days, or from about three to about five days. Suitably a single microbial strain is cultivated. Suitably, the method comprises a single cultivation step.

In some embodiments, at least about 50 wt %, at least about 60 wt %, at least about 70 wt %, at least about 75 wt %, or at least about 80 wt % of the polyester waste is utilised during the cultivation. In some embodiments, at least about 0.01 mg/ml, at least about 0.02 mg/ml, at least about 0.03 mg/ml, at least about 0.04 mg/ml, at least about 0.05 mg/ml, or at least about 0.1 mg/ml PHA is produced. In some embodiments, at least about 10 μg PHA/mg dry cell weight (DCW), at least about 20 μg PHA/mg DCW, at least about 30 μg PHA/mg DCW, at least about 40 μg PHA/mg DCW, or at least about 50 μg PHA/mg DCW is produced. The PHA may comprise or consist of polyhydroxybutyrate (PHB) or a co-polymer thereof and/or polyhydroxyvalerate (PHV) or a co-polymer thereof. In some embodiments, the PHA comprises or consists of polyhydroxybutyrate (PHB) or a co-polymer thereof. In some embodiments, the PHA comprises or consists of poly(3-hydroxybutyrate-co-3-hydroxyvalerate) (PHBV).

The method may further comprise any other suitable steps. Suitably, the method further comprises a step of recovering the PHA.

In another aspect, the present invention provides a culture broth comprising polyester waste and a microbe, wherein the microbe is capable of utilising a plurality of polyester monomers from the polyester waste to produce PHA.

In another aspect, the present invention provides a polyhydroxyalkanoate (PHA) produced by the method according to the present invention.

In another aspect, the present invention provides an article comprising or consisting of the PHA produced by the method according to the present invention. The article may be packaging.

In another aspect, the present invention provides use of a microbe for producing polyhydroxyalkanoate (PHA) from polyester waste, wherein the microbe is capable of utilising a plurality of polyester monomers from the polyester waste to produce PHA.

In another aspect, the present invention provides a microbe for producing polyhydroxyalkanoate (PHA) from polyester waste, wherein the microbe is capable of utilising a plurality of polyester monomers from the polyester waste to produce PHA. The microbe may have been genetically engineered to express at least part of one or more of the pathways.

In another aspect, the present invention provides a vector comprising a gene encoding an enzyme for producing polyhydroxyalkanoate (PHA) from polyester waste.

In another aspect, the present invention provides a cell comprising the vector according to the present invention.

### BRIEF DESCRIPTION OF THE DRAWINGS

Additional features and advantages of the present invention are described in, and will be apparent from, the description of the presently preferred embodiments which are set out below with reference to the drawings in which:

FIG. 1 is a schematic showing the microbial recycling of polyester-based plastic waste and their related monomers into polyhydroxyalkanoates (PHAs) by Paracoccus denitrificans based on its genetic capabilities.

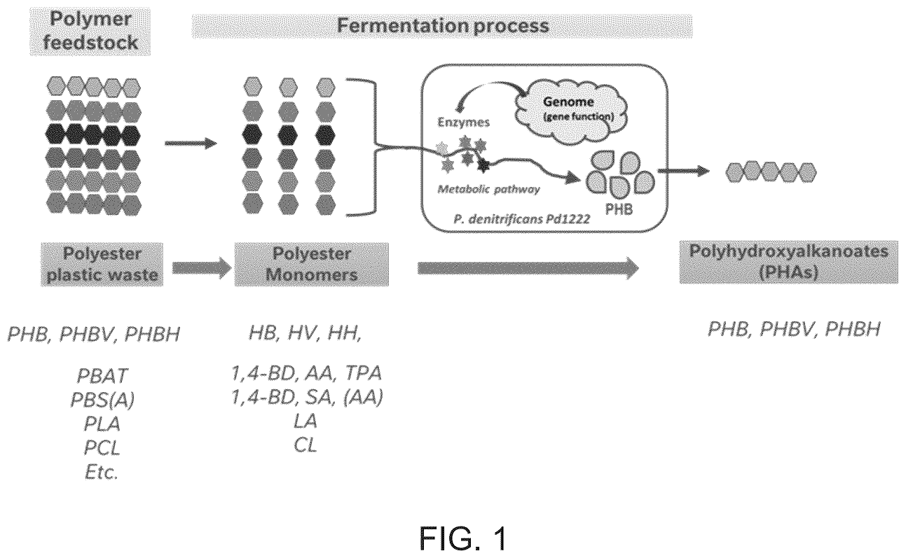

FIG. 2 is a visualization of the metabolic capabilities for conversion of polyester monomers by Paracoccus denitrificans based on a constructed GSM (Genome Scale Model). Metabolic routes identified for the conversion of different polyester monomers to PHA (PHB) by Paracoccus denitrificans and its associated genes and enzymes respectively. Metabolic routes for utilization of monomers from polymers building blocks: A) Connected to central carbon metabolism (TCA cycle) for PBS (succinic acid), PLA (lactic acid), PHV (3-Hydroxyvaleric acid), PBAT (adipic acid), hydroxycaproic acid, PHB (3 hydroxy butyric acid), PBS (1,4 butanediol). Arrows also indicating the route for production of PHB from the central carbon metabolism (TCA cycle) B) metabolic route for assimilation of PET (ethylene glycol).

FIG. 3 shows the biomass formation (cell mass dry weight) of Paracoccus denitrificans when supplying different monomers as sole carbon source as in 0.3% (w/v) of the media.

FIG. 4 shows the amount of poly(hydroxy butyrate-co-valerate) (PHBV) produced by P. denitrificans using the different plastic monomers as sole carbon source.

FIG. 5 shows A) cell growth of P. denitrificans and B) consumption of plastic monomer and production of PHB under anoxic conditions.

FIG. 6 shows A) cell growth of P. denitrificans and B) production of PHB from mechanically and chemically pretreated polymers.

### DETAILED DESCRIPTION OF THE INVENTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise. The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around.

Unless otherwise indicated, any nucleic acid sequences are written left to right in 5′ to 3′ orientation; amino acid sequences are written left to right in amino to carboxy orientation, respectively.

All publications mentioned in the specification are herein incorporated by reference. The publications discussed herein are provided solely for their disclosure prior to the filing date of the present application. Nothing herein is to be construed as an admission that such publications constitute prior art to the claims appended hereto.

Method for Producing PHA from Polymer Waste

In one aspect, the present invention provides a method for producing polyhydroxyalkanoate (PHA) from polyester waste, the method comprising the steps of: (a) providing a culture broth comprising polyester waste; and (b) cultivating a microbe in the culture broth to produce PHA.

Microbe for Producing PHA from Polyester Waste

Any suitable microbe described herein (e.g. in the section entitled “Microbe”) may be used to produce the PHA from the polyester waste. A mixture of microbes may be used or a single microbe. In some embodiments, a single microbe (e.g. a single microbial strain) is used.

The microbe may utilise one or more polyester monomer from the polyester waste to produce the PHA. Suitably, the microbe utilises two or more, three or more, four or more, five or more, six or more, or seven or more polyester monomers from the polyester waste to produce the PHA. The polyester monomers may be in the form of free polyester monomers, oligoesters, or polyesters. Suitably, the polyester monomers are in the form of free polyester monomers or oligoesters. Suitably, the polyester monomers are in the form of free polyester monomers.

In some embodiments, the microbe utilises one or more, two or more, three or more, four or more, five or more, six or more, or seven or more polyester monomers from the polyester waste selected from: succinic acid, lactic acid, ethylene glycol, adipic acid, 6-hydroxycaproic acid, 3-hydroxybutyric acid, 3-hydroxyvaleric acid, and 1,4-butanediol to produce the PHA. In some embodiments, the microbe utilises each of succinic acid, lactic acid, ethylene glycol, adipic acid, 6-hydroxycaproic acid, 3-hydroxybutyric acid, 3-hydroxyvaleric acid, and 1,4-butanediol from the polyester waste to produce the PHA.

The microbe may utilises polyester monomers from one or more polyester from the polyester waste to produce the PHA. Suitably, the microbe is capable of utilising polyester monomers from two or more, three or more, four or more, five or more, six or more, seven or more, or eight or more polyesters from the polyester waste to produce the PHA.

In some embodiments, the microbe utilises polyester monomers from one or more, two or more, three or more, four or more, five or more, six or more, seven or more, or eight or more polyesters from the polyester waste selected from: polybutylene succinate (PBS), polybutylene succinate adipate (PBSA), poly(butylene succinate-co-terephthalate) (PBST), poly(butylene succinate/terephthalate/isophthalate)-co-(lactate) (PBSTIL), polybutylene terephthalate (PBT), polybutylene adipate terephthalate (PBAT), polyethylene terephthalate (PET), poly(ethylene adipate) (PEA), polylactic acid (PLA), polycaprolactone (PCL), polyhydroxybutyrate (PHB), poly(3-hydroxybutyrate-co-3-hydroxyvalerate) (PHBV), and poly(3-hydroxybutyrate-co-3-hydroxyhexanoate) (PHBH) to produce the PHA.

In some embodiments, the microbe utilises polyester monomers from one or more, two or more, three or more, four or more, five or more, six or more, or seven polyesters selected from: polybutylene succinate (PBS), polybutylene adipate terephthalate (PBAT), polylactic acid (PLA), polycaprolactone (PCL), polyhydroxybutyrate (PHB), poly(3-hydroxybutyrate-co-3-hydroxyvalerate) (PHBV), and poly(3-hydroxybutyrate-co-3-hydroxyhexanoate) (PHBH) to produce the PHA. In some embodiments, the microbe utilises polyester monomers from one or more, two or more, or three polyesters selected from: polyhydroxybutyrate (PHB), poly(3-hydroxybutyrate-co-3-hydroxyvalerate) (PHBV), and poly(3-hydroxybutyrate-co-3-hydroxyhexanoate) (PHBH) to produce the PHA.

In some embodiments, the microbe utilises at least about 20 wt %, at least about 30 wt %, at least about 40 wt %, at least about 50 wt %, at least about 60 wt %, at least about 70 wt %, at least about 75 wt %, at least about 80 wt %, at least about 85 wt %, at least about 90 wt %, at least about 95 wt % of the polyester waste.

### Polyester Waste

Any suitable polyester waste may be used. As described above, the present inventors have shown that the method allows the utilisation of a wide variety of polyester monomers, including polyester monomers that are difficult for biological metabolism, such as 1,4-butanediol. The method allows the utilisation of mixed polyester waste comprising biodegradable polyesters (e.g. PHA, PHB, PHBH) as well as non-biodegradable polyesters (e.g. PET). The polyester waste may be polyester plastic waste.

Polyesters are polymers that contain the ester functional group in every repeat unit of their main chain. Polyesters may include naturally occurring polymers as well as synthetic polymers. Natural polyesters and a few synthetic ones are biodegradable, but most synthetic polyesters are not biodegradable. Polyesters include polyethylene terephthalate (PET), polytrimethylene terephthalate (PTT), polybutylene terephthalate (PBT), polyethylene isosorbide terephthalate (PEIT), polylactic acid (PLA), polyhydroxyalkanoates (PHA) such as polyhydroxybutyrate (PHB) and poly(3-hydroxybutyrate-co-3-hydroxyvalerate) (PHBV), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polybutylene succinate terephthalate (PBST), polyethylene succinate (PES), and poly(butylene succinate/terephthalate/isophthalate)-co-(lactate) (PBSTIL).

Polyesters are synthesised from polyester monomers. For example: PET may be synthesised from ethylene glycol and terephthalic acid; PTT may be synthesised from 1,3-propanediol and terephthalic acid; PBT may be synthesised from 1,4-butanediol and terephthalic acid; PLA may be synthesised from lactic acid; PHB may be synthesised from 3-hydroxybutyric acid; PHBV may be synthesised from 3-hydroxybutyric acid and 3-hydroxyvaleric acid; PBS may be synthesised from succinic acid and 1,4-butanediol; PBSA may be synthesised from succinic acid, 1,4-butanediol, and adipic acid; PBAT may synthesised from 1,4-butanediol and adipic acid; PEF may be synthesised from 2,5-furandicarboxylic acid and ethylene glycol; PCL may be synthesised from 6-hydroxycaproic acid; PEA may be synthesised from adipic acid and ethylene glycol; PBST may be synthesised from succinic acid, terephthalic acid and 1,4-butanediol; PES may be synthesised from ethylene glycol and succinic acid; and PBSTIL may be synthesised from succinic acid, lactic acid, 1,4-butanediol, terephthalic acid, and isophthalic acid. Conversely polyester polymers may be degraded to their polyester monomers e.g. by hydrolytic cleavage of the ester bonds. Hydrolytic cleavage may occur passively or can be catalysed by chemical processes or enzymatic processes.

Suitably, the polyester waste comprises one or more, two or more, three or more, four or more, five or more, six or more, or seven or more polyester monomers. In preferred embodiments, the polyester waste comprises a plurality of polyester monomers. The polyester monomers may be in the form of free polyester monomers, oligoesters, or polyesters. Suitably, the polyester monomers are in the form of free polyester monomers or oligoesters. Suitably, the polyester monomers are in the form of free polyester monomers. The present inventors have shown that the method of the invention allows either free polyester monomers, oligomers or polymers (e.g. following a pre-treatment step) to be utilised.

In some embodiments, the polyester waste comprises one or more, two or more, three or more, four or more, five or more, six or more, or seven or more polyester monomers selected from succinic acid, lactic acid, ethylene glycol, adipic acid, 6-hydroxycaproic acid, 3-hydroxybutyric acid, 3-hydroxyvaleric acid, and 1,4-butanediol. In preferred embodiments, the polyester waste comprises 1,4-butanediol. In some embodiments, the polyester waste comprises each of succinic acid, lactic acid, ethylene glycol, adipic acid, 6-hydroxycaproic acid, 3-hydroxybutyric acid, 3-hydroxyvaleric acid, and 1,4-butanediol (in the form of free polyester monomers or in the form of polyester polymers).

Suitably, the polyester waste comprises one or more, two or more, three or more, four or more, five or more, six or more, seven or more, or eight or more polyesters. In preferred embodiments, the polyester waste comprises a plurality of polyesters.

In some embodiments, the polyester waste comprises one or more, two or more, three or more, four or more, five or more, six or more, seven or more, or eight or more polyesters selected from: polybutylene succinate (PBS), polybutylene succinate adipate (PBSA), poly(butylene succinate-co-terephthalate) (PBST), poly(butylene succinate/terephthalate/isophthalate)-co-(lactate) (PBSTIL), polybutylene terephthalate (PBT), polybutylene adipate terephthalate (PBAT), polyethylene terephthalate (PET), poly(ethylene adipate) (PEA), polylactic acid (PLA), polycaprolactone (PCL), polyhydroxybutyrate (PHB), and poly(3-hydroxybutyrate-co-3-hydroxyvalerate) (PHBV), poly(3-hydroxybutyrate-co-3-hydroxyhexanoate) (PHBH), or copolymers thereof.

In some embodiments, the polyester waste comprises one or more, two or more, three or more, four or more, five or more, six or more, or seven polyesters selected from: polybutylene succinate (PBS), polybutylene adipate terephthalate (PBAT), polylactic acid (PLA), polycaprolactone (PCL), polyhydroxybutyrate (PHB), poly(3-hydroxybutyrate-co-3-hydroxyvalerate) (PHBV), and poly(3-hydroxybutyrate-co-3-hydroxyhexanoate) (PHBH). In some embodiments, the polyester waste comprises one or more, two or more, or three polyesters selected from: polyhydroxybutyrate (PHB), poly(3-hydroxybutyrate-co-3-hydroxyvalerate) (PHBV), and poly(3-hydroxybutyrate-co-3-hydroxyhexanoate) (PHBH).

### Pre-Treatment

The polyester waste may be pre-treated, i.e. treated prior to adding to the culture broth. The pre-treated polyester waste may comprise polyester monomers and/or oligomers, particularly polyester monomers. The present inventors have shown that pre-treatment may promote degradation of the polyester waste into smaller particles and make the polyester monomers more accessible for utilisation. Any suitable pre-treatment may be used (see e.g. Ragaert, K., et al., 2017. Waste management, 69, pp. 24-58 and WO2002036675A2). Any combination of pre-treatment steps may be used, and each step may be repeated one or more times.

In some embodiments, the method of the present invention comprises the steps of: (a) pre-treating a polyester waste; (b) providing a culture broth comprising the pre-treated polyester waste; and (c) cultivating a microbe in the culture broth to produce PHA. In some embodiments, the polyester waste is mechanically treated and/or chemically treated (prior to adding to the culture broth).

In some embodiments, the polyester waste is mechanically treated (prior to adding to the culture broth). For example, the polyester waste may be separated and/or sorted; bailed; washed; ground, shredded and/or cut; and/or compounded and/or pelletized prior to adding to the culture broth.

In some embodiments, the polyester waste is separated and/or sorted (prior to adding to the culture broth). This may occur based on shape, density, size, colour or chemical composition. The polyester waste may be manually sorted or automatically sorted (e.g. by flotation). In some embodiments, the polyester waste is bailed (prior to adding to the culture broth). If the plastic is not processed where it is sorted, it is often bailed in between for transport purposes. In some embodiments, the polyester waste is washed (prior to adding to the culture broth). Washing may be used to remove contaminants, such as organic contaminants.

In preferred embodiments, the polyester waste is ground, shredded and/or cut (prior to adding to the culture broth). This may reduce the size of the polyester waste, e.g. to produce flakes. In some embodiments the polyester waste is shredded (prior to adding to the culture broth). Suitably, the polyester waste may have a particle size of from about 0.1 mm to about 20 mm, from about 0.2 mm to about 10 mm, from about 0.3 mm to about 5 mm, from about 0.4 mm to about 2 mm, or from about 0.5 mm to about 1 mm. Suitably, the polyester waste may have a particle size of from about 100 μm to about 5000 μm, from about 200 μm to about 4000 μm, from about 300 μm to about 3000 μm, from about 400 μm to about 2000 μm, or from about 500 μm to about 1000 μm.

In some embodiments, the polyester waste is chemically treated (prior to adding to the culture broth). Chemical treatment may be used to partially or completely depolymerise the polyester into its polyester monomers. Different depolymerization routes such as methanolysis, glycolysis, hydrolysis, ammonolysis, aminolysis, and/or hydrogenation may be used depending on the chemical agent and polyester. In some embodiments, the polyester waste is hydrolysed (prior to adding to the culture broth). During hydrolysis the polyester may be reacted with water under neutral or acidic conditions to break the polyester chains. High temperatures and/or pressures may be used to accelerate hydrolysis. In some embodiments, the polyester waste undergoes alkaline treatment (prior to adding to the culture broth). Suitably, the polyester waste may be incubated in an alkaline solution (e.g. about 0.5M to about 2M NaOH or about 1M to about 2M NaOH) at about 30 to about 40° C. (e.g. about 37° C.) for about 5 to about 20 days (e.g. about 7 to about 15 days), optionally with shaking or stirring at about 200 rpm to about 500 rpm (e.g. about 300 rpm to about 400 rpm). Suitably, the polyester waste is neutralised following the alkaline treatment. Suitably, the polyester waste is neutralised with an acidic solution (e.g. containing hydrochloric acid). Suitably, the polyester waste is neutralised to about pH 7.

In some embodiments, the polyester waste is mechanically treated and chemically treated (prior to adding to the culture broth). In some embodiments, the polyester waste is ground, shredded and/or cut and undergoes alkaline treatment (prior to adding to the culture broth).

### Culture Broth

In one aspect, the present invention provides a culture broth comprising polyester waste and a microbe. The microbe may be any suitable microbe described herein for producing PHA from polyester waste (e.g. in the section entitled “Microbe”). The polyester waste may be any polyester waste described herein (e.g. in the sub-section entitled “polyester waste”). Suitably, the culture broth may further comprise PHA, suitably any PHA described herein (e.g. in the sub-section entitled “production of PHA”).

The polyester waste may be added to the culture broth in any suitable amount. Suitably, the culture broth comprises the polyester waste in an amount of at least about 1 g/L, at least about 2 g/L, at least about 3 g/L, at least about 4 g/L, or at least about 5 g/L. Suitably, the culture broth comprises the polyester waste in an amount of about 100 g/L or less, about 90 g/L or less, about 80 g/L or less, about 70 g/L or less, about 60 g/L or less, about 50 g/L or less, about 40 g/L or less, about 30 g/L or less, about 25 g/L or less, about 20 g/L or less, about 15 g/L or less, about 10 g/L or less, about 9 g/L or less, about 8 g/L or less, about 7 g/L or less, about 6 g/L or less, or about 5 g/L or less. Suitably, the culture broth comprises the polyester waste in an amount of from about 1 g/L to about 100 g/L, from about 1 g/L to about 90 g/L, from about 1 g/L to about 80 g/L, from about 1 g/L to about 70 g/L, from about 1 g/L to about 60 g/L, from about 1 g/L to about 50 g/L, from about 1 g/L to about 40 g/L, from about 1 g/L to about 30 g/L, from about 1 g/L to about 25 g/L, from about 1 g/L to about 20 g/L, from about 2 g/L to about 10 g/L, or from about 2 g/L to about 5 g/L.

Any suitable culture broth may be used to cultivate the microbe. Suitably, the culture broth is about pH 7 and comprise all the nutrients and trace elements necessary to cultivate the microbe. The culture broth may depend on the microbe and/or culture conditions used. For example, an optimum culture broth for anaerobic growth of Paracoccus denitrificans is described in Hahnke, S. M., et al., 2014. Frontiers in microbiology, 5, p.18.

In some embodiments, the culture broth comprises mineral salt medium. Suitably, the culture broth comprises mineral salt medium in an amount of at least about 80% (v/v), at least about 85% (v/v), at least about 90% (v/v), or at least about 95% (v/v). Suitably, the mineral salt medium may comprise about 22.7 g/L dipotassium hydrogen phosphate, about 0.95 g/L potassium dihydrogen phosphate, about 0.67 g/L ammonium sulfate and about 2 ml/L trace metals solution. Suitably, a trace metals solution may comprise sodium, zinc, calcium, iron, molybdenum, copper, cobalt, manganese, and magnesium

### Cultivation Conditions

Any suitable cultivation conditions may be used. As described above, the present inventors have shown that the method may proceed via a one-step process, carrying out cultivation using a single microbe. The present inventors have also shown that the cultivation can be carried out under aerobic or anoxic conditions. The culture conditions may depend on the microbe used.

Suitably, the microbe is cultivated under aerobic or anoxic conditions. As used herein, “aerobic” conditions are rich in free oxygen (O₂). As used herein, “anoxic” conditions are characterised by limited free oxygen or a lack of free oxygen but containing other electron acceptors (e.g. ammonium sulfate, potassium nitrate, and/or sodium sulfate). Suitably, anoxic conditions may be generated by replacing oxygen with another gas, for example an inert gas (e.g. helium). Suitably, anoxic conditions may be generated by helium washing. In some embodiments, the microbe is cultivated under aerobic conditions. In some embodiments, the microbe is cultivated under anoxic conditions. The microbe may cultivated partly under aerobic conditions and partly under anoxic conditions.

The microbe may cultivated for any suitable duration. Suitably, the microbe is cultivated for at least about one day, at least about two days, at least about three days, at least about four days, or at least about five days. Suitably, the microbe is cultivated for about 10 days or less, about 9 days or less, about 8 days or less, about 7 days or less, about 6 days or less, or about 5 days or less. Suitably, the microbe is cultivated for from about one to about seven days, from about two to about six days, or from about three to about five days. Suitably, the microbe is cultivated until a cell biomass of at least about 0.1 mg/ml, at least about 0.2 mg/ml, at least about 0.3 mg/ml, at least about 0.4 mg/ml, or at least about 0.5 mg/ml is reached.

The microbe may cultivated at any suitable temperature. For example, Paracoccus denitrificans may be cultivated at temperature from 11-45° C. (Hahnke, S. M., et al., 2014. Frontiers in microbiology, 5, p.18). Suitably, the microbe is cultivated at from about 30° C. to about 40° C. Suitably, the microbe is cultivated at about 30° C., about 31° C., about 32° C., about 33° C., about 34° C., about 35° C., about 36° C., or at about 37° C. Suitably, the microbe is cultivated at about 30° C., about 34° C., or at about 37° C.

The microbe may be cultivated under static, shaking, or stirring conditions. In some embodiments, the microbe is cultivated under shaking or stirring conditions. In some embodiments, the microbe is cultivated under shaking conditions. Any suitable shaking conditions may be used (see e.g. Klöckner, W. and Büchs, J., 2012. Trends in biotechnology, 30 (6), pp. 307-314). Suitably, the shaking conditions may be from about 100 rpm to about 400 rpm, or from about 200 rpm to about 300 rpm.

The cultivation broth may be inoculated with the microbe. Any suitable inoculation may be used. Suitably, inoculum is added in an amount of at least about 1% (v/v) or at least about 2% (v/v). Suitably, inoculum is added in an amount of about 1% (v/v) or about 2% (v/v). Suitably, the inoculum may be derived from a cultivation of the microbe in a rich medium (e.g. LB medium).

The method may comprise one or more cultivation steps. In some embodiments, the method comprises a single cultivation step. In some embodiments, the method does not comprise a second cultivation step with a second microbe (or a second mixture of microbes). During a single cultivation step, the cultivation conditions may be adjusted, for example to decrease or increase the temperature and/or the culture conditions may be monitored and maintained, e.g. to maintain the pH and/or minimum level of nutrients. A single cultivation step may consist of cultivating a single microbe (or a single mixture of microbes) in the culture broth. A further cultivation step may consist of cultivating another microbe (or another mixture of microbes) in the culture broth.

### Production of PHA

The type and/or quantity of PHA produced may depend on e.g. the polyester waste utilised, the microbe, and/or the cultivation conditions. The skilled person would be able to optimise the method to achieve the desired type and/or quantity of PHA.

Polyhydroxyalkanoates (PHAs) comprise a group of natural biodegradable polyesters that are synthesized by microorganisms and may have the following general molecular formula where, typically x=1-8 and n=100 to 1000 (see Li, Z., et al., 2016. NPG Asia Materials, 8 (4), pp.e265-e265):

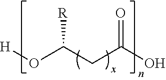

More than 150 different PHA monomers have been identified, which renders them the largest group of natural polyester. Commonly synthesized PHA monomers include: 3-hydroxybutyrate, 3-hydroxyvalerate, 3-hydroxyhexanoate, 3-hydroxyoctanoate, 3-hydroxydecanoate, 3-hydroxydodecanoate. Exemplary PHAs include poly(3-hydroxybutyrate) (PHB), poly(3-hydroxyvalerate) (PHV), poly(3-hydroxybutyrate-co-3-hydroxyvalerate) (PHBV), poly(3-hydroxybutyrate-co-3-hydroxyhexanoate) (PHBH), and poly(3-hydroxyoctanoate) (PHO), poly(3-hydroxynonanoate) (PHN), and their copolymers with 3-hydroxyhexanoate (HHx), 3-hydroxyheptanoate (HH) and/or 3-hydroxydecanoate (HD).

The PHA produced by the present invention will vary depending on the polyester waste. For example, if the polyester waste is rich in polyester monomers such as lactic acid, succinic acid, ethylene glycol, adipic acid, 3-hydroxybutyric acid, 6-hydroxycaproic acid, and/or 1,4-butanediol, then the PHA produced may be rich in 3-hydroxybutyrate monomers. For example, if the polyester waste is rich in 3-hydroxyvaleric acid, then the PHA produced may be rich in 3-hydroxyvalerate monomers.

The PHA produced by the present invention may comprise or consist of PHAs comprising 3-hydroxybutyrate monomers, 3-hydroxyvalerate monomers, and/or 3-hydroxyhexanoate monomers. The PHA produced by the present invention may comprise or consist of PHAs comprising 3-hydroxybutyrate monomers and/or 3-hydroxyvalerate monomers. The PHA produced by the present invention may comprise or consist of PHAs comprising 3-hydroxybutyrate monomers and/or 3-hydroxyvalerate monomers. The PHA produced by the present invention may comprise 3-hydroxybutyrate monomers. In some embodiments, the PHA produced by the present invention comprises or consists of from about 90% to about 95% (e.g. about 90%, about 91%, about 92%, about 93%, about 94%, or about 95%) 3-hydroxybutyrate monomers and from about 5% to about 10% (e.g. about 5%, about 6%, about 7%, about 8%, about 9%, or about 10%) 3-hydroxyvalerate monomers. In other embodiments, the PHA produced by the present invention comprises or consists of from about 5% to about 10% (e.g. about 5%, about 6%, about 7%, about 8%, about 9%, or about 10%) 3-hydroxybutyrate monomers and from about 90% to about 95% (e.g. about 90%, about 91%, about 92%, about 93%, about 94%, or about 95%) 3-hydroxyvalerate monomers, for example when the polyester waste is rich in 3-hydroxyvaleric acid.

The PHA produced by the present invention may comprise or consist of poly(3-hydroxybutyrate) (PHB), poly(3-hydroxyvalerate) (PHV), and poly(3-hydroxybutyrate-co-3-hydroxyvalerate) (PHBV). The PHA produced by the present invention may comprise or consist of poly(3-hydroxybutyrate) (PHB) or a co-polymer thereof and/or poly(3-hydroxyvalerate) (PHV) or a copolymer thereof. The PHA produced by the present invention may comprise or consist of poly(3-hydroxybutyrate) (PHB) or a co-polymer thereof. PHB co-polymers may include poly(3-hydroxybutyrate-co-3-hydroxyvalerate) (PHBV) and/or poly(3-hydroxybutyrate-co-3-hydroxyhexanoate) (PHBH).

The PHA produced by the present invention may comprise or consist of poly(3-hydroxybutyrate-co-3-hydroxyvalerate) (PHBV). In some embodiments, the PHBV comprises or consists of from about 90% to about 95% (e.g. about 90%, about 91%, about 92%, about 93%, about 94%, or about 95%) 3-hydroxybutyrate and from about 5% to about 10% (e.g. about 5%, about 6%, about 7%, about 8%, about 9%, or about 10%) 3-hydroxyvalerate. In other embodiments, the PHBV comprises or consists of from about 5% to about 10% (e.g. about 5%, about 6%, about 7%, about 8%, about 9%, or about 10%) 3-hydroxybutyrate monomers and from about 90% to about 95% (e.g. about 90%, about 91%, about 92%, about 93%, about 94%, or about 95%) 3-hydroxyvalerate monomers.

The method of the present invention may be used to produce PHA in an amount of at least about 0.01 mg/ml, at least about 0.02 mg/ml, at least about 0.03 mg/ml, at least about 0.04 mg/ml, at least about 0.05 mg/ml, or at least about 0.1 mg/ml. The method of the present invention may be used to produce PHA in an amount of at least about 10 μg PHA/mg dry cell weight (DCW), at least about 20 μg PHA/mg DCW, at least about 30 μg PHA/mg DCW, at least about 40 μg PHA/mg DCW, or at least about 50 μg PHA/mg DCW.

Recovery, Separation and/or Purification of PHA

Once the cultivation step has concluded, the PHA may be recovered, separated and/or purified from the resulting microbial slurry using any suitable method known in the art (see e.g. Pagliano, G., et al., 2021. Frontiers in Bioengineering and Biotechnology, 9, p.54; Pérez-Rivero, C., et al., 2019. Biochemical Engineering Journal, 150, p.107283; and López-Abelairas, M., et al., 2015. Biochemical Engineering Journal, 93, pp. 250-259).

In some embodiments, the method of the present invention comprises the steps of: (a) providing a culture broth comprising polyester waste; (b) cultivating a microbe in the culture broth to produce a microbial slurry comprising PHA; and (c) recovering the PHA from the microbial slurry. In some embodiments, the method of the present invention comprises the steps of: (a) pre-treating a polyester waste; (b) providing a culture broth comprising the pre-treated polyester waste; (c) cultivating a microbe in the culture broth to produce a microbial slurry comprising PHA; and (d) recovering the PHA from the microbial slurry. The PHA may be recovered by solvents and/or by cell lysis.

Suitably, the PHA may be recovered from the microbial slurry by solvent extraction (see e.g. Pagliano, G., et al., 2021. Frontiers in Bioengineering and Biotechnology, 9, p.54). Solvent extraction of PHA from the microbial slurry may involve the following steps: (i) contacting and mixing the microbial slurry with solvent; (ii) heating up the mixture; (iii) separating the extraction residues (non-PHA biomass and water) from the PHA-enriched phase (PHA dissolved in the solvent); and (iv) separating PHA from the solvent by evaporation of PHA precipitation.

Suitably, the PHA may be recovered from the microbial slurry by cell lysis action (see e.g. Pagliano, G., et al., 2021. Frontiers in Bioengineering and Biotechnology, 9, p.54). Cell lysis may involve the following steps: (i) mixing the microbial slurry and an additive (e.g. alkali, surfactant, oxidant) to solubilise non-PHA constituents; (ii) separating solid PHA from the liquid (containing the non-PHA constituents); and (iii) drying and purifying the PHA.

Once recovered, the PHA may be subjected to one or more further downstream processing steps, such as separation and/or purification (see e.g. Pérez-Rivero, C., et al., 2019. Biochemical Engineering Journal, 150, p.107283). In some embodiments, the PHA is separated. Conventional separation methods include sedimentation, crystallization, centrifugation, decantation, filtration or a combination thereof. In some embodiments, the PHA is purified. Suitably, PHA may be purified by washing crude PHA with a solvent e.g. with ethanol, acetone, diethyl ether, or any combination thereof.

In some embodiments, the method of the present invention comprises the steps of: (a) providing a culture broth comprising polyester waste; (b) cultivating a microbe in the culture broth to produce a microbial slurry comprising PHA; (c) recovering the PHA from the microbial slurry to provide a crude PHA; and (d) separating and/or purifying the crude PHA to provide purified PHA. In some embodiments, the method of the present invention comprises the steps of: (a) pre-treating a polyester waste; (b) providing a culture broth comprising the pre-treated polyester waste; (c) cultivating a microbe in the culture broth to produce a microbial slurry comprising PHA; (d) recovering the PHA from the microbial slurry to provide a crude PHA; and (e) separating and/or purifying the crude PHA to provide purified PHA.

### PHA and Articles

In one aspect, the present invention provides a polyhydroxyalkanoate (PHA) obtained by or obtainable by the method according to the present invention.

The PHA may comprise or consist of poly(3-hydroxybutyrate-co-3-) hydroxyvalerate) (PHBV). PHBV is the most promising biopolymer for petroleum-based plastics replacement, the low processes productivity as well as the high sale price represent a major barrier for its widespread usage. In some embodiments, the PHBV comprises or consists of from about 90% to about 95% (e.g. about 90%, about 91%, about 92%, about 93%, about 94%, or about 95%) 3-hydroxybutyrate and from about 5% to about 10% (e.g. about 5%, about 6%, about 7%, about 8%, about 9%, or about 10%) 3-hydroxyvalerate. In other embodiments, the PHBV comprises or consists of from about 5% to about 10% (e.g. about 5%, about 6%, about 7%, about 8%, about 9%, or about 10%) 3-hydroxybutyrate monomers and from about 90% to about 95% (e.g. about 90%, about 91%, about 92%, about 93%, about 94%, or about 95%) 3-hydroxyvalerate monomers.

The PHA may be provided in any suitable form, for example, the PHA may be provided in the form of a resin, sealant, adhesive, granules, powder, microbeads, spheres, sheets, films, pellets, etc.

In one aspect, the present invention provides an article comprising or consisting of a polyhydroxyalkanoate (PHA) produced by the method according to the present invention.

PHA may have a wide range of applications. For example, as packaging materials for food and other perishable goods (see e.g. Bugnicourt, E., et al., 2014. eXPRESS Polymer Letters Vol.8, No.11 (2014) 791-808); in the medical and pharmaceutical field (see e.g. Valappil, S. P., et al., 2006. Expert Review of Medical Devices, 3 (6), pp. 853-868); and in the transportation sector, e.g., in automobiles or aircraft. Applications include packaging, moulded goods, paper coatings, non-woven fabrics, adhesives, films and performance additives

Suitably, the PHA produced by the method according to the present invention may be used as packaging material, plastic bags, cutlery, and food containers. In some embodiments, the article of the present invention is packaging, films, and/or bags. For example, the article of the present invention may be food packaging, fresh film, mulching film, laminating film, wrapping film, heat-shrinkable film, shopping bags, garbage bags, gift bags, and produce bags. In some embodiments, the article of the present invention is a vial, a bottle, or a container.

### Microbe

In one aspect, the present invention provides a microbe for producing polyhydroxyalkanoate (PHA) from polyester waste. The microbe may be isolated from its natural environment or produced by means of a technical process (e.g. genetic engineering).

As used herein, a “microbe” or “microorganism” may refer to an organism of microscopic size, which may exist in its single-celled form or as a colony of cells. Exemplary microbes include bacteria, archaea, fungi and protists.

Suitably, the microbe of the present invention may be a bacteria. Suitably, the microbe of the present invention is from the family Rhodobacteraceae. Suitably, the microbe of the present invention is from the genus Paracoccus. Species in the genus Paracoccus may include P. acridae, P. aeridis, P. aerius, P. aestuarii, P. aestuariivivens, P. alcaliphilus, P. alimentarius, P. alkanivorans, P. alkenifer, P. aminophilus, P. aminovorans, P. amoyensis, P. angustae, P. aquimaris, P. aurantiacus, P. baruchii, P. beibuensis, P. binzhouensis, P. bogoriensis, P. caeni, P. carotinifaciens, P. cavernae, P. chinensis, P. communis, P. contaminans, P. denitrificans, P. endophyticus, P. ferrooxidans, P. fistulariae, P. fontiphilus, P. gahaiensis, P. haematequi, P. haeundaensis, P. halophilus, P. halotolerans, P. hibisci, P. hibiscisoli, P. homiensis, P. huijuniae, P. indicus, P. isoporae, P. jeotgali, P. kamogawaensis, P. kawasakiensis, P. kocurii, P. kondratievae, P. koreensis, P. laeviglucosivorans, P. liaowanqingii, P. lichenicola, P. limosus, P. litorisediminis, P. luteus, P. lutimaris, P. mangrovi, P. marcusii, P. marinus, P. methylutens, P. mutanolyticus, P. niistensis, P. nototheniae, P. oceanense, P. onubensis, P. pacificus, P. panacisoli, P. pantotrophus, P. pueri, P. ravus, P. rhizosphaerae, P. salipaludis, P. saliphilus, P. sanguinis, P. sediminilitoris, P. sediminis, P. seriniphilus, P. shandongensis, P. siganidrum, P. simplex, P. solventivorans, P. sordidisoli, P. speluncae, P. sphaerophysae, P. stylophorae, P. subflavus, P. sulfuroxidans, P. suum, P. tegillarcae, P. thiocyanatus, P. thiophilus, P. tibetensis, P. versutus, P. xiamenensis, P. yeei, P. zeaxanthinifaciens, and P. zhejiangensis.

In some embodiments, the microbe of the present invention is a Paracoccus denitrificans, Paracoccus pantotrophus, or Paracoccus versutus. In some embodiments, the microbe of the present invention is a Paracoccus denitrificans or a Paracoccus pantotrophus.

In some embodiments, the microbe of the present invention is a Paracoccus denitrificans. Paracoccus denitrificans is a gram-negative, coccus, non-motile, denitrifying (nitrate-reducing) bacterium (Kelly, D. P. et al., 2006. International Journal of systematic and evolutionary microbiology, 56 (10), pp. 2495-2500).

In some embodiments, the microbe of the present invention is Paracoccus denitrificans DSM 413, Paracoccus denitrificans PD1222, Paracoccus denitrificans CNCM I-5881, Paracoccus denitrificans ATCC 19367, Paracoccus denitrificans ATCC 17741, Paracoccus denitrificans ATCC 13543, Paracoccus denitrificans NCIB 8944, Paracoccus denitrificans NRRL B-3785, Paracoccus denitrificans CCM 982, Paracoccus denitrificans LMD 22.21, Paracoccus denitrificans JCM 21484, Paracoccus denitrificans NBRC 102528, Paracoccus denitrificans NCCB 22021, Paracoccus denitrificans NBRC 13301, Paracoccus denitrificans NCIMB 8944, Paracoccus denitrificans DSM 15418, Paracoccus denitrificans DSM 415, Paracoccus denitrificans NCIMB 11627, Paracoccus denitrificans NCIMB 9722, Paracoccus denitrificans IMET 10380, Paracoccus denitrificans VKM B-1324, Paracoccus denitrificans ICPB 3979, or a derivative thereof.

In some embodiments, the microbe of the present invention is Paracoccus denitrificans DSM 413, Paracoccus denitrificans PD1222, Paracoccus denitrificans CNCM I-5881, Paracoccus denitrificans ATCC 19367, Paracoccus denitrificans ATCC 17741, Paracoccus denitrificans ATCC 13543, Paracoccus denitrificans NCIB 8944, Paracoccus denitrificans NRRL B-3785, Paracoccus denitrificans CCM 982, Paracoccus denitrificans LMD 22.21, Paracoccus denitrificans JCM 21484, Paracoccus denitrificans NBRC 102528, Paracoccus denitrificans NCCB 22021, or a derivative thereof.

In some embodiments, the microbe of the present invention is Paracoccus denitrificans DSM 413, Paracoccus denitrificans PD1222, Paracoccus denitrificans CNCM I-5881, or a derivative thereof.

In preferred embodiments, the microbe of the present invention is Paracoccus denitrificans DSM 413, or a derivative thereof. Paracoccus denitrificans DSM 413 was deposited at the German Collection of Microorganisms and Cell Cultures GmbH (DSMZ) before 22 Aug. 1990 and is a type strain. Derivatives of Paracoccus denitrificans DSM 413 may include Paracoccus denitrificans PD1222, Paracoccus denitrificans CNCM I-5881, Paracoccus denitrificans ATCC 19367, Paracoccus denitrificans ATCC 17741, Paracoccus denitrificans ATCC 13543, Paracoccus denitrificans NCIB 8944, Paracoccus denitrificans NRRL B-3785, Paracoccus denitrificans CCM 982, Paracoccus denitrificans LMD 22.21, Paracoccus denitrificans JCM 21484, Paracoccus denitrificans NBRC 102528, and Paracoccus denitrificans NCCB 22021.

Comparison of 16S rRNA gene sequences is routinely used to ensure the correct placement of strains of Paracoccus within the alpha-3 subgroup of the class Alphaproteobacteria (Kelly, D. P. et al., 2006. International Journal of systematic and evolutionary microbiology, 56 (10), pp. 2495-2500). Suitably, the 16S rRNA gene sequence of the microbe has at least 95%, at least 96%, at least 97%, at least 98.0%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, at least 99.8%, at least 99.9%, or 100.0% identity to the 16S rRNA gene sequence of Paracoccus denitrificans DSM 413 (see e.g. GenBank accession no. Y16929.1).

In some embodiments, the microbe of the present invention is Paracoccus denitrificans PD1222, or a derivative thereof. Paracoccus denitrificans PD1222 (NCBI: txid318586) is a derivative of DSM 413 and is model soil microorganism able to perform the complete denitrification pathway (Baker, S.C., et al., 1998. Microbiology and Molecular Biology Reviews, 62 (4), pp. 1046-1078). Paracoccus denitrificans PD1222 may also be known as Paracoccus denitrificans NCCB 97099 (Kelly, D. P. et al., 2006. International Journal of systematic and evolutionary microbiology, 56 (10), pp. 2495-2500).

In some embodiments, the microbe of the present invention is the Paracoccus denitrificans deposited by SOCIÉTÉ DES PRODUITS NESTLÉ at the Collection Nationale de Cultures de Microorganismes (CNCM) (Institut Pasteur, 25-28, rue du Docteur Roux, 75724 Paris Cedex 15) in accordance with the terms of the Budapest Treaty on 12 Sep. 2022, under the number CNCM I-5881. The deposited strain may be referred to herein as Paracoccus denitrificans CNCM I-5881.

In some embodiments, the microbe of the present invention has at least 98.0%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, at least 99.8%, at least 99.9%, or 100.0% sequence identity to the Paracoccus denitrificans having GenBank assembly accession no. GCA_000203895.1.

As used herein, a “derivative” of an existing strain may refer to a genetically engineered variant (e.g. in which one or more gene has been knocked-out and/or inserted) or a naturally-occurring variant (e.g. in which genetic drift has occurred). Typically, a derivative may have at least 98.0%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, at least 99.8%, at least 99.9%, or 100.0% sequence identity to the original strain.

### Utilisation of Polyester Waste

The microbe of the present invention may be capable of utilising one or more polyester monomers to produce PHA. Suitably, the microbe is capable of utilising two or more, three or more, four or more, five or more, six or more, or seven or more polyester monomers to produce PHA.

In some embodiments, the microbe is capable of utilising one or more, two or more, three or more, four or more, five or more, six or more, or seven or more polyester monomers selected from: succinic acid, lactic acid, ethylene glycol, adipic acid, 6-hydroxycaproic acid, 3-hydroxybutyric acid, 3-hydroxyvaleric acid, and 1,4-butanediol to produce PHA. In some embodiments, the microbe is capable of utilising each of succinic acid, lactic acid, ethylene glycol, adipic acid, 6-hydroxycaproic acid, 3-hydroxybutyric acid, 3-hydroxyvaleric acid, and 1,4-butanediol to produce PHA.

The microbe may comprise genes encoding for one or more pathway for the utilisation of polyester monomers. Suitably, the microbe comprises genes encoding for two or more pathways, three or more pathways, four or more pathways, five or more pathways, six or more pathways, or seven or more pathways for the utilisation of polyester monomers.

In some embodiments, the microbe comprises genes encoding for two or more pathways, three or more pathways, four or more pathways, five or more pathways, six or more pathways, or seven or more pathways selected from: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol.

In some embodiments, the microbe comprises genes encoding for each of: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol.

In preferred embodiments, the microbe comprises genes encoding a pathway for the utilisation of 1,4-butanediol. Suitably, the microbe comprises one or more gene encoding a methanol dehydrogenase, an aldehyde dehydrogenase, an alcohol dehydrogenase, and/or a succinate-semialdehyde dehydrogenase. In some embodiments, the microbe comprises one or more of: (i) a gene encoding a methanol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 31; (ii) a gene encoding a methanol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 33; (iii) a gene encoding an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 23; (iv) a gene encoding an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 35; (v) a gene encoding an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 37; (vi) a gene encoding an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 5; (vii) a gene encoding a succinate-semialdehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 39; (viii) a gene encoding a succinate-semialdehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 41; and (ix) a gene encoding a succinate-semialdehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 43. In some embodiments, the microbe comprises one or more of: (i) a gene encoding a methanol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 32; (ii) a gene encoding a methanol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 34; (iii) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 24; (iv) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 36; (v) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 38; (vi) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 6; (vii) a gene encoding a succinate-semialdehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 40; (viii) a gene encoding a succinate-semialdehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 42; and (ix) a gene encoding a succinate-semialdehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 44.

In some embodiments, the microbe comprises genes encoding a pathway for the utilisation of succinic acid. Suitably, the microbe comprises one or more gene encoding a succinate dehydrogenase. In some embodiments, the microbe comprises: (i) a gene encoding a succinate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 1. In some embodiments, the microbe comprises: (i) a gene encoding a succinate dehydrogenase and having least 70% sequence identity to the nucleotide sequence of SEQ ID NO: 2.

In some embodiments, the microbe comprises genes encoding a pathway for the utilisation of lactic acid. Suitably, the microbe comprises one or more gene encoding a D-lactate dehydrogenase. In some embodiments, the microbe comprises: (i) a gene encoding a D-lactate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 3. In some embodiments, the microbe comprises: (i) a gene encoding a D-lactate dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 4.

In some embodiments, the microbe comprises genes encoding a pathway for the utilisation of ethylene glycol. Suitably, the microbe comprises one or more gene encoding an alcohol dehydrogenase, an aldehyde dehydrogenase, and a glyoxylate reductase. In some embodiments, the microbe comprises one or more of: (i) a gene encoding an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 5; (ii) a gene encoding an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 7; and (iii) a gene encoding a glyoxylate reductase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 9. In some embodiments, the microbe comprises one or more of: (i) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 6; (ii) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 8; and (iii) a gene encoding a glyoxylate reductase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 10.

In some embodiments, the microbe comprises genes encoding a pathway for the utilisation of adipic acid. Suitably, the microbe comprises one or more gene encoding a long-chain-fatty-acid-CoA ligase, an acyl-CoA dehydrogenase, an enoyl-CoA hydratase, a 3-hydroxybutyryl-CoA dehydrogenase, and a 3-oxoadipyl-CoA thiolase. In some embodiments, the microbe comprises one or more of: (i) a gene encoding a long-chain-fatty-acid-CoA ligase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 11; (ii) a gene encoding an acyl-CoA dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 13; (iii) a gene encoding an enoyl-CoA hydratase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 15; (iv) a gene encoding a 3-hydroxybutyryl-CoA dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 17; and (v) a gene encoding a 3-oxoadipyl-CoA thiolase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 19. In some embodiments, the microbe comprises one or more of: (i) a gene encoding a long-chain-fatty-acid-CoA ligase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 12; (ii) a gene encoding an acyl-CoA dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 14; (iii) a gene encoding an enoyl-CoA hydratase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 16; (iv) a gene encoding a 3-hydroxybutyryl-CoA dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 18; and (v) a gene encoding a 3-oxoadipyl-CoA thiolase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 20.

In some embodiments, the microbe comprises genes encoding a pathway for the utilisation of 6-hydroxycaproic acid. Suitably, the microbe comprises one or more gene encoding an alcohol dehydrogenase and an aldehyde dehydrogenase. In some embodiments, the microbe comprises: (i) a gene encoding an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 21; and/or (ii) a gene encoding an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 23. In some embodiments, the microbe comprises: (i) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 22; and/or (ii) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 24.

In some embodiments, the microbe comprises genes encoding a pathway for the utilisation of 3-hydroxybutyric acid. Suitably, the microbe comprises one or more gene encoding an Acyl-CoA synthetase and a 3-hydroxybutyrate dehydrogenase.

In some embodiments, the microbe comprises: (i) a gene encoding an Acyl-CoA synthetase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 25; and/or (ii) a gene encoding a 3-hydroxybutyrate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 27. In some embodiments, the microbe comprises: (i) a gene encoding an Acyl-CoA synthetase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 26; and/or (ii) a gene encoding a 3-hydroxybutyrate dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 28.

In some embodiments, the microbe comprises genes encoding a pathway for the utilisation of 3-hydroxyvaleric acid. Suitably, the microbe comprises one or more gene encoding an Acyl-CoA synthetase, a 3-hydroxybutyrate dehydrogenase, and a 3-ketoacyl-CoA thiolase. In some embodiments, the microbe comprises one or more of: (i) a gene encoding an Acyl-CoA synthetase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 25; (ii) a gene encoding a 3-hydroxybutyrate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 27; and (iii) a gene encoding a 3-ketoacyl-CoA thiolase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 29. In some embodiments, the microbe comprises one or more of: (i) a gene encoding an Acyl-CoA synthetase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 26; (ii) a gene encoding a 3-hydroxybutyrate dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 28; and (iii) a gene encoding a 3-ketoacyl-CoA thiolase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 30.

The microbe may comprise genes encoding for one or more pathway for the synthesis of PHA. At present, a total of at least 14 pathways have been reported which lead to PHA synthesis. Suitably, the microbe comprises one or more gene encoding a PHA synthase. PHA synthase is the key enzyme involved in PHA biosynthesis and functions by polymerizing monomeric hydroxyalkanoate substrates. PHA synthases have been categorized into four major classes based on their primary sequences, substrate specificity, and subunit composition. Any suitable PHA synthase may be used, for example a natural PHA synthase or a genetically engineered PHA synthase (Chek, M. F., et al., 2017. Scientific reports, 7 (1), pp. 1-15).

Suitably, the microbe comprises one or more gene encoding a 3-ketoacyl-CoA thiolase, an acetoacetyl-CoA reductase and a PHA synthase. Suitably, the microbe comprises one or more gene encoding a 3-ketoacyl-CoA thiolase, an enoyl-CoA hydratase, and a PHA synthase.

In some embodiments, the microbe comprises one or more of: (i) a gene encoding a 3-ketoacyl-CoA thiolase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 45; (ii) a gene encoding an enoyl-CoA hydratase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 47; and (iii) a gene encoding a PHA synthase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 49. In some embodiments, the microbe comprises one or more of: (i) a gene encoding a 3-ketoacyl-CoA thiolase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 46; (ii) a gene encoding an enoyl-CoA hydratase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 48; and (iii) a gene encoding a PHA synthase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 50.

The microbe of the present invention may be capable of utilising polyester monomers from one or more polyester to produce PHA. Suitably, the microbe is capable of utilising polyester monomers from two or more, three or more, four or more, five or more, six or more, seven or more, or eight or more polyesters to produce PHA.

In some embodiments, the microbe is capable of utilising polyester monomers from one or more, two or more, three or more, four or more, five or more, six or more, seven or more, eight or more, nine or more, ten or more, eleven or more, or twelve or more polyesters selected from: polybutylene succinate (PBS), polybutylene succinate adipate (PBSA), poly(butylene succinate-co-terephthalate) (PBST), poly(butylene succinate/terephthalate/isophthalate)-co-(lactate) (PBSTIL), polybutylene terephthalate (PBT), polybutylene adipate terephthalate (PBAT), polyethylene terephthalate (PET), poly(ethylene adipate) (PEA), polylactic acid (PLA), polycaprolactone (PCL), polyhydroxybutyrate (PHB), poly(3-hydroxybutyrate-co-3-hydroxyvalerate) (PHBV), and poly(3-hydroxybutyrate-co-3-hydroxyhexanoate) (PHBH) to produce PHA. In some embodiments, the microbe is capable of utilising polyester monomers from each of PBS, PBSA, PBST, PBSTIL, PBT, PBAT, PET, PEA, PLA, PCL, PHB, PHBV, and PHBH, to produce PHA.

### Genetically Engineered Microbe

In some embodiments, the microbe has been genetically engineered. The microbe may be genetically engineered to improve the utilisation of one or more polyester monomer and/or to improve the synthesis of PHA. This may be achieved by e.g. introducing genes encoding part or all of a pathway, or by optimising promoters and/or RBSs to increase the expression of part or all of a pathway (see e.g. Zhang, X., et al., 2020. Trends in biotechnology, 38 (7), pp. 689-700). In some embodiments, the microbe is genetically engineered to overexpress all part or all of a pathway.

The microbe may be genetically engineered by any suitable method (see e.g. Keasling, J. D., 1999. Trends in biotechnology, 17 (11), pp. 452-460 and Yan, Q. and Fong, S. S., 2017. Frontiers in microbiology, 8, p.2060). Suitably, the microbe is genetically engineered by transfection, by transduction, or by gene-editing.

In some embodiments, the microbe is genetically engineered by transfection. The term “transfection” or “transformation” may refer to a type of genetic engineering in which a non-viral vector is used to deliver a gene to a target cell. Typical transformation methods for bacteria may use plasmid DNA.

In some embodiments, the microbe is genetically engineered by transduction. The term “transduction” may refer to a type of genetic engineering in which a viral vector is used to deliver a gene to a target cell. Typical transduction method for bacteria may use a bacteriophage.

In some embodiments, the microbe is genetically engineered by gene-editing. The term “gene editing” may refer to a type of genetic engineering in which a nucleic acid is inserted, deleted or replaced in a cell. Gene editing may be achieved using engineered nucleases, which may be targeted to a desired site in a polynucleotide (e.g. a genome). Such nucleases may create site-specific double-strand breaks at desired locations, which may then be repaired through non-homologous end-joining (NHEJ) or homologous recombination (HR), resulting in targeted mutations. Such nucleases may be delivered to a target cell using vectors. Examples of suitable nucleases known in the art include zinc finger nucleases (ZFNs), transcription activator like effector nucleases (TALENs), and the clustered regularly interspaced short palindromic repeats (CRISPR)/Cas system (see e.g. Gaj, T. et al. (2013) Trends Biotechnol. 31:397-405).

In some embodiments, the microbe has been genetically engineered to express, to enhance expression of, and/or to overexpress at least part of one or more of the pathways described. In some embodiments, the microbe has been genetically engineered to express, to enhance expression of, and/or to overexpress at least part of one or more pathway for the utilisation of polyester monomers and/or the microbe has been genetically engineered to express, to enhance expression of, and/or to overexpress at least part of one or more pathway for the synthesis of PHA.

In some embodiments, the microbe has been genetically engineered to express, to enhance expression of, and/or to overexpress at least part of a pathway for the utilisation of succinic acid. Suitably, the microbe has been genetically engineered to express, to enhance expression of, and/or to overexpress one or more gene encoding a succinate dehydrogenase. In some embodiments, the microbe has been genetically engineered to express, to enhance expression of, and/or to overexpress a succinate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 1. In some embodiments, the microbe has been genetically engineered to introduce a gene encoding a succinate dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 2.

In some embodiments, the microbe has been genetically engineered to express, to enhance expression of, and/or to overexpress at least part of a pathway for the utilisation of lactic acid. Suitably, the microbe has been genetically engineered to express, to enhance expression of, and/or to overexpress one or more gene encoding a D-lactate dehydrogenase. In some embodiments, the microbe has been genetically engineered to express, to enhance expression of, and/or to overexpress a D-lactate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 3. In some embodiments, the microbe has been genetically engineered to introduce a gene encoding a D-lactate dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 4.

In some embodiments, the microbe has been genetically engineered to express, to enhance expression of, and/or to overexpress at least part of a pathway for the utilisation of ethylene glycol. Suitably, the microbe has been genetically engineered to express, to enhance expression of, and/or to overexpress one or more gene encoding an alcohol dehydrogenase, an aldehyde dehydrogenase, and/or a glyoxylate reductase. In some embodiments, the microbe has been genetically engineered to express, to enhance expression of, and/or to overexpress one or more of: (i) a gene encoding an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 5; (ii) a gene encoding an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 7; and (iii) a gene encoding a glyoxylate reductase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 9. In some embodiments, the microbe has been genetically engineered to introduce one or more of: (i) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 6; (ii) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 8; and (iii) a gene encoding a glyoxylate reductase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 10.

In some embodiments, the microbe has been genetically engineered to express, to enhance expression of, and/or to overexpress at least part of a pathway for the utilisation of adipic acid. Suitably, the microbe has been genetically engineered to express, to enhance expression of, and/or to overexpress one or more gene encoding a long-chain-fatty-acid-CoA ligase, an acyl-CoA dehydrogenase, an enoyl-CoA hydratase, a 3-hydroxybutyryl-CoA dehydrogenase, and/or a 3-oxoadipyl-CoA thiolase. In some embodiments, the microbe has been genetically engineered to express, to enhance expression of, and/or to overexpress one or more of: (i) a long-chain-fatty-acid-CoA ligase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 11; (ii) an acyl-CoA dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 13; (iii) an enoyl-CoA hydratase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 15; (iv) a 3-hydroxybutyryl-CoA dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 17; and (v) a 3-oxoadipyl-CoA thiolase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 19. In some embodiments, the microbe has been genetically engineered to introduce one or more of: (i) a gene encoding a long-chain-fatty-acid-CoA ligase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 12; (ii) a gene encoding an acyl-CoA dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 14; (iii) a gene encoding an enoyl-CoA hydratase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 16; (iv) a gene encoding a 3-hydroxybutyryl-CoA dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO:

18; and (v) a gene encoding a 3-oxoadipyl-CoA thiolase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 20.

In some embodiments, the microbe has been genetically engineered to express, to enhance expression of, and/or to overexpress at least part of a pathway for the utilisation of 6-hydroxycaproic acid. Suitably, the microbe has been genetically engineered to express, to enhance expression of, and/or to overexpress one or more gene encoding an alcohol dehydrogenase and/or an aldehyde dehydrogenase. In some embodiments, the microbe has been genetically engineered to express, to enhance expression of, and/or to overexpress: (i) an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 21; and/or (ii) an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 23. In some embodiments, the microbe has been genetically engineered to introduce: (i) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 22; and/or (ii) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 24.

In some embodiments, the microbe has been genetically engineered to express, to enhance expression of, and/or to overexpress at least part of a pathway for the utilisation of 3-hydroxybutyric acid. Suitably, the microbe has been genetically engineered to express, to enhance expression of, and/or to overexpress one or more gene encoding an Acyl-CoA synthetase, and/or a 3-hydroxybutyrate dehydrogenase. In some embodiments, the microbe has been genetically engineered to express, to enhance expression of, and/or to overexpress: (i) an Acyl-CoA synthetase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 25; and/or (ii) a a 3-hydroxybutyrate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 27. In some embodiments, the microbe has been genetically engineered to introduce: (i) a gene encoding an Acyl-CoA synthetase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 26; and/or (ii) a gene encoding a 3-hydroxybutyrate dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 28.

In some embodiments, the microbe has been genetically engineered to express, to enhance expression of, and/or to overexpress at least part of a pathway for the utilisation of 3-hydroxyvaleric acid. Suitably, the microbe has been genetically engineered to express, to enhance expression of, and/or to overexpress one or more gene encoding an Acyl-CoA synthetase, a 3-hydroxybutyrate dehydrogenase, and/or a 3-ketoacyl-CoA thiolase. In some embodiments, the microbe has been genetically engineered to express, to enhance expression of, and/or to overexpress one or more of: (i) an Acyl-CoA synthetase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 25; (ii) a 3-hydroxybutyrate dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 27; and (iii) a 3-ketoacyl-CoA thiolase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 29. In some embodiments, the microbe has been genetically engineered to introduce one or more of: (i) a gene encoding an Acyl-CoA synthetase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 26; (ii) a gene encoding a 3-hydroxybutyrate dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 28; and (iii) a gene encoding a 3-ketoacyl-CoA thiolase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 30.

In some embodiments, the microbe has been genetically engineered to express, to enhance expression of, and/or to overexpress at least part of a pathway for the utilisation of 1,4-butanediol. Suitably, the microbe has been genetically engineered to express, to enhance expression of, and/or to overexpress one or more gene encoding a methanol dehydrogenase, an aldehyde dehydrogenase, an alcohol dehydrogenase, and/or a succinate-semialdehyde dehydrogenase. In some embodiments, the microbe has been genetically engineered to express, to enhance expression of, and/or to overexpress one or more of: (i) a methanol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 31; (ii) a methanol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 33; (iii) an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 23; (iv) an aldehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 35; (v) an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 37; (vi) an alcohol dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 5; (vii) a succinate-semialdehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 39; (viii) a succinate-semialdehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 41; and (ix) a succinate-semialdehyde dehydrogenase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 43. In some embodiments, the microbe has been genetically engineered to introduce one or more of: (i) a gene encoding a methanol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 32; (ii) a gene encoding a methanol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 34; (iii) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 24; (iv) a gene encoding an aldehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 36; (v) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 38; (vi) a gene encoding an alcohol dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 6; (vii) a gene encoding a succinate-semialdehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 40; (viii) a gene encoding a succinate-semialdehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 42; and (ix) a gene encoding a succinate-semialdehyde dehydrogenase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 44.

In some embodiments, the microbe has been genetically engineered to express, to enhance expression of, and/or to overexpress at least part of a pathway for the synthesis of a PHA. Suitably, the microbe has been genetically engineered to express, to enhance expression of, and/or to overexpress one or more gene encoding a 3-ketoacyl-CoA thiolase, an enoyl-CoA hydratase, and/or a PHA synthase. In some embodiments, the microbe has been genetically engineered to express, to enhance expression of, and/or to overexpress one or more of: (i) a 3-ketoacyl-CoA thiolase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 45; (ii) an enoyl-CoA hydratase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 47; and (iii) a PHA synthase having at least 70%, at least 75%, at least 80%, at least 85%, at least 90%, at least 95%, at least 96%, at least 97%, at least 98%, at least 99% or 100% sequence identity to the amino acid sequence of SEQ ID NO: 49. In some embodiments, the microbe has been genetically engineered to introduce one or more of: (i) a gene encoding a 3-ketoacyl-CoA thiolase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 46; (ii) a gene encoding an enoyl-CoA hydratase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 48; and (iii) a gene encoding a PHA synthase and having at least 70%, at least 75%, at least 80%, at least 85%, at least 90%, at least 95%, at least 96%, at least 97%, at least 98%, at least 99% or 100% sequence identity to the nucleotide sequence of SEQ ID NO: 50.

### Vector

In one aspect, the present invention provides a vector comprising a gene encoding an enzyme for producing polyhydroxyalkanoate (PHA) from polyester waste.

A “vector” is a tool that allows or facilitates the transfer of an entity from one environment to another. In accordance with the present invention, and by way of example, some vectors used in recombinant nucleic acid techniques allow entities, such as a segment of nucleic acid, to be transferred into a target cell. The vector may serve the purpose of maintaining the heterologous nucleic acid within the cell, facilitating the replication of the vector comprising a segment of nucleic acid, or facilitating the expression of the protein encoded by a segment of nucleic acid.

Vectors may be non-viral or viral. Examples of vectors used in recombinant nucleic acid techniques include, but are not limited to, plasmids, cosmids, chromosomes, artificial chromosomes and viruses. The vector may be single stranded or double stranded. The vector may be a naked nucleic acid (e.g. DNA). The vectors used in the invention may be, for example, a naked nucleic acid, plasmid or viral vectors.

In one embodiment, the vector is a plasmid. A “plasmid” may refer to a small, extrachromosomal DNA molecule within a cell that is physically separated from chromosomal DNA and can replicate independently. Plasmids are most commonly found as small circular, double-stranded DNA molecules in bacteria, however, plasmids are sometimes present in archaea and eukaryotic organisms.

In one embodiment, the vector is a viral vector. Viral vectors were originally developed as an alternative to transfection of naked DNA for molecular genetics experiments. Compared to traditional methods of transfection, transduction efficiency can be higher and some viruses integrate into the cell genome facilitating stable expression. Suitably, the viral vector is a bacteriophage.

### Succinic Acid Pathway

In one embodiment, the vector comprises one or more genes encoding all or part of a pathway for the utilisation of succinic acid. Suitably, the vector comprises a gene encoding a succinate dehydrogenase.

In one embodiment, the vector comprises a gene encoding a succinate dehydrogenase. Succinate dehydrogenase (EC.1.3.5.1) is an enzyme that may catalyse the oxidation of succinate to fumarate. In one embodiment, the vector comprises a gene encoding a succinate dehydrogenase having at least 70%, at least 75%, at least 80%, at least 85%, at least 90%, at least 95%, at least 96%, at least 97%, at least 98%, at least 99% or 100% sequence identity to SEQ ID NO: 1. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 2.

(SEQ ID NO: 1)
MPKPDLPDLPDTCDLLVVGSGAAGMAAAISAHHHGLKPVIVEKSE
FFGGSTAVSGGAIWVPCNPIAAAAGMTDDREAARAYIRGETGNRF
NAELVDAFLDKSPEAIGFFHERTALKMAHRALSPDYHSDAPGATE
GGRALDALDYDGRRLGADLYRMRPPIADFTILGGMPLGRPDIFHF
LRMTRSVKSAAYATGAVLRYFRDRLTWGRNTRLVMGAAVSGRLAE
TVFARNIPLFTGHELVRLLQDESGRVVGAELKGPRGVCRIAAHRG
VVLAAGGYPHDAARRAQSFEHVRRGLPHYSMSPVSGTGGGIAAAE
AVGAAFVDTNPNAGFWTPVSLLRNADGSVRPFPHLFLDRAKPGVI
AVGHDGRRFVNEASSYHDFVQGLIAKLLADGEKSAWLVADHRAMR
RYGLGAAHAFPARIGRHVASGYLKRDATLEGLARQCGIDVATFRQ
TVALFNEAAARGEDPAFGKGSTSYQRYLGDGENRPNPCLRPLEGP
FYAVEIYPGDIGTSMGLDITAKGEVRDSRGRTIPGLYACGNDINS
VMSGAYPGPGITLGPALTFGYVIGQSAAA
Exemplary succinate dehydrogenase
(SEQ ID NO: 2)
atgccgaagcctgatctgccggacctgcccgatacctgcgacctt
ctggtcgtgggttcgggtgccgcgggcatggcggcggcgatctct
gcccatcatcacgggttgaagccggtcatcgtcgagaagtccgaa
ttcttcggcggctccaccgccgtttccggcggcgcgatctgggtg
ccgtgcaatccgatcgccgcggcggccggcatgaccgacgaccgc
gaggcggcgcgcgcctatatccggggcgagacgggcaatcgcttc
aatgccgagctggtggacgcctttctcgacaagagccccgaggcc
atcggctttttccacgaaagaaccgcgctcaagatggcgcatcgc
gcgctgtcgcccgactatcattcggatgcgccgggtgcgaccgaa
ggcggccgggcgctggacgcgctggattatgacggccgccggctg
ggggcggacctttatcggatgcgcccgcccatcgccgatttcacg
atcctcggcggaatgccgctggggcgcccggacatctttcacttc
ctgcgcatgacccggtcggtgaaatcggcggcctatgcgaccggg
gcagtgctgcgctatttccgcgaccggctgacctgggggcggaac
acgcggctggtgatgggcgcggcggtctcggggcgcctggccgag
acggtgttcgcaaggaacatcccgctttttaccgggcatgagctg
gtgcggctgctgcaggacgaatccgggcgggtcgtcggggccgaa
ctcaaggggccacggggcgtttgccggatcgcggcgcatcgcggc
gtggttcttgcggcgggcggctatccgcatgatgcggcgcgtcgg
gcgcaaagcttcgagcatgtcaggcgcggcctgccgcattattcg
atgtcgccggtctcgggcaccggcggcgggatcgccgcggccgag
gccgtcggagcggcctttgtcgacaccaaccccaatgccggtttc
tggacgccggtttcgctcttgcgcaatgccgacggatcggtgcgg
cccttcccgcatcttttcctggaccgtgccaagcccggcgtgatc
gccgtcggccatgacggccggcgcttcgtcaacgaggcttccagc
tatcatgacttcgtgcagggattgatcgcgaagctgctggccgac
ggagagaaatccgcctggctggtcgccgatcaccgggcgatgcgg
cgctatgggctgggtgccgcgcatgcctttccggcgcggatcggc
cggcatgtcgccagcggctatctgaagcgcgacgcgacgctggag
gggctggcccggcaatgcggcatcgacgtcgcgacgttccggcag
acggtcgccctgttcaacgaggcggcggcacgcggcgaagacccg
gccttcggcaagggatcgaccagctatcagcgctatctgggcgat
ggcgagaaccggccgaacccctgcctgcgcccgctggaggggccg
ttctacgccgtcgagatctatccgggcgacatcggcacctctatg
ggcctggacatcacggcgaaaggcgaggtcagggacagccggggc
aggacgatacccggcctctatgcctgcggcaacgacatcaattcg
gtgatgtccggcgcctatcccgggccgggcatcacgctgggtccg
gcgctgaccttcggatatgtcatcgggcaatcggcggccgcatga
Exemplary succinate dehydrogenase gene-peg.3652

### Lactic Acid Pathway

In one embodiment, the vector comprises one or more genes encoding all or part of a pathway for the utilisation of lactic acid. Suitably, the vector comprises a gene encoding a D-lactate dehydrogenase.

In one embodiment, the vector comprises a gene encoding a D-lactate dehydrogenase. D-lactate dehydrogenase (EC 1.1.1.28) is an enzyme that may catalyse the conversion of lactate into pyruvate. In one embodiment, the vector comprises a gene encoding a D-lactate dehydrogenase having at least 70%, at least 75%, at least 80%, at least 85%, at least 90%, at least 95%, at least 96%, at least 97%, at least 98%, at least 99% or 100% sequence identity to SEQ ID NO: 3. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 4.

(SEQ ID NO: 3)
MTRPGQAEAAADRTALLDRLRRIVGPAHVLTADRATRRYTRGFRY
GEGPVAAVVRPGSLVQMWRVLNAAVASGRAVILQAANTGLIGGST
PWGQDYDREIVLVSVMRLRGIHLIGAGEQVLCLPGATLDALEKRL
RPLGREPHSVIGSSCIGASVLGGICNNSGGALIQRGPAYTEMSLY
AEVREDGSVALVNHLGLDLGDDPEEILARVEAGELPAPAPTDAWA
SDREYADHVRDIEAETPARFNADPRRLHESSGCAGKLAVFAVRLD
TFQAEKDTAVFYVGSNDPDELTEIRRHILAHFQSLPIAGEYIHRE
AYDIAAKYGKDTFLFIRHAGTDRMPAFFAAKARMDALTERLGLGA
TLSDRLAQGVAALMPQHLPRRMNDFRDRYEHHLLLRMGGAGIAEA
RDYLGAIFPSASGAMFECTPDEGKAAFLHRFAVAGAAVRYRAIHA
REVQDIVALDIALRRNDRDWVERLPPDLDAKLEKKLYYGHFFCHV
FHQDYVVKKGQDCLAVEHEMWRLLDRRGAEYPAEHNVGHLYHAKP
ELAGFYRQLDPINSLNPGIGQTSKCAHWH
Exemplary D-lactate dehydrogenase
(SEQ ID NO: 4)
atgacgcgacccggccaggccgaggcagccgcagaccgcaccgca
ttgctggaccggctgcgccggatcgtgggaccggcgcatgtgctg
accgccgaccgcgccacccgccgctatacccgcggcttccgctat
ggcgaggggccggtggccgcggtggtccgccccggctctctggtc
cagatgtggcgggtcctgaacgccgccgtcgcctcggggcgcgcg
gtgatcctgcaggccgcgaataccgggctgaccggcggctcgacc
ccctggggccaggattacgaccgcgagatcgtgctggtctcggtc
atgcgcctgcgcggcatccacctcatcggcgcgggcgaacaggtg
ctctgcctgcccggcgccacgctcgacgcgctggaaaagcgcctg
cgccccctgggacgagagccgcattcggtcatcggctcgtcctgc
atcggcgcctctgtcctgggcggcatctgcaacaattccggcggc
gcgctgatccagcgcggcccggcctataccgagatgagcctttac
gccgaggtgcgcgaggacggctcggtggcgctggtcaaccatctg
ggcctcgacctcggcgacgatcccgaggaaatcctggcccgcgtc
gaggcgggcgaattgcccgcccccgcgcccaccgacgcctgggcc
tcggaccgcgaatatgccgaccatgtccgcgacatcgaggccgag
accccggcccgcttcaacgccgatccgcgccggctgcacgaatcc
tcgggctgcgccggcaagctcgcagtctttgccgtgcggctggac
acgttccaggccgaaaaggacacggcggtcttctatgtcggcagc
aacgacccggacgagttgaccgagatccgccgccatatcctggcg
catttccagagcctgcccatcgccggcgaatacatccaccgcgaa
gcctatgacatcgcagcgaaatacggcaaggacaccttcctgttc
atccgccatgccggcaccgaccgcatgccggcattcttcgccgcc
aaggcgcgcatggacgcgctgaccgagcggctggggctgggcgcc
accctctcggaccggctggcgcagggcgtcgcggcgctgatgccc
cagcacctgcccaggcgcatgaacgatttccgcgaccgctacgaa
caccacctgctgctgcgcatgggcggcgccggcatcgccgaggcg
cgcgactatctcggcgccatcttcccctcggccagcggcgccatg
ttcgaatgcacccccgacgagggcaaggccgccttcctgcaccgt
ttcgccgtggcgggcgccgccgtgcgctatcgcgccatccacgcc
cgcgaggtgcaggacatcgtggcgctggacatcgcgctgcgccgc
aacgaccgcgactgggtcgagcgcctgccgccggacctggacgcg
aaactcgagaaaaagctctattacggccatttcttctgccacgtc
ttccatcaggactacgtggtgaaaaagggccaggactgcctggcc
gtcgagcatgagatgtggcgcctcttggaccggcgcggcgccgaa
tacccggccgagcacaatgtcggccacctttaccacgccaagccg
gaactggccggcttctaccggcagctggacccgaccaacagcctg
aaccccggcatcggccagacctcgaaatgcgcgcattggcactag
Exemplary D-lactate dehydrogenase gene-peg.3013

### Ethylene Glycol Pathway

In one embodiment, the vector comprises one or more genes encoding all or part of a pathway for the utilisation of ethylene glycol. Suitably, the vector comprises a gene encoding an alcohol dehydrogenase, an aldehyde dehydrogenase, and/or a glyoxylate reductase.

In one embodiment, the vector comprises a gene encoding an alcohol dehydrogenase. Alcohol dehydrogenase (EC 1.1.1.1) is an enzyme that may catalyse the conversion of alcohol into an aldehyde or ketone (e.g. converts ethylene glycol into glycolaldehyde). In one embodiment, the vector comprises a gene encoding an alcohol dehydrogenase having at least 70%, at least 75%, at least 80%, at least 85%, at least 90%, at least 95%, at least 96%, at least 97%, at least 98%, at least 99% or 100% sequence identity to SEQ ID NO: 5. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 6.

(SEQ ID NO: 5)
MTFRANWSYPTTIKFGPGRVTELAEHCRAVGIARPLLVTDKALAS
LPITAQALDVLDASGLGRAVFSEVDPNPHEGNMEAGIAAYKAGGH
DGVICFGGGSALDLGKMIALMADQTVSVWDLEDIGDWWTRADAGK
IAPIIAVPTTAGTGSEVGRAGVLINSATHKKKIIFHPRLMPAVTI
CDPELTVGMPKFITAGTGMDAFAHCLEAFCSPHYHPMSQGIALEG
LRLVNEYLPRAYATPDDLEARAHMMSAAAMGAVAFQKGLGAIHSL
SHPVGAVYGTHHGITNAVVMPMVLDFNRSAIEDRLARAADYLGIK
GGFDGFRARVIQLRSELAIPQNLTRLGVQTERLDELTEMALEDPS
CGGNPVEMTRENTRALFESCM
Exemplary alcohol dehydrogenase
(SEQ ID NO: 6)
atgacgtttcgtgcgaactggtcctatccgaccaccatcaaattc
ggcccgggccgcgtcaccgaactggccgagcattgcagggccgtg
ggcatcgcgcgtccgctgctggtgaccgacaaggcattggcgagc
ctgcccataaccgcgcaggccctggatgttctggatgcctccggc
cttggccgcgcggtcttttccgaggtcgatccgaacccgcacgaa
ggcaatatggaggccggcatcgccgcctacaaagccggcggacat
gacggcgtgatctgctttggcggcggctcggcgctggatctgggc
aagatgatcgcgctgatggcggaccagaccgtatccgtttgggac
ttggaggacatcggcgactggtggacccgcgcggatgccggcaag
atcgcgcccatcattgccgtgccgacgaccgcgggcacgggctcc
gaggtcgggcgcgccggggtcctgacgaactcggccacgcacaag
aagaagatcatcttccaccccaggctgatgcctgccgtcacgatc
tgcgaccccgaactgaccgtgggcatgccgaaattcatcaccgcc
ggcaccggcatggatgccttcgcccattgcctcgaagcgttctgc
tcgccccattaccaccccatgtcgcagggcatcgcgcttgagggg
ctgcggctggtcaacgaatacctgccccgcgcctatgccacgccc
gacgacctcgaggcccgcgcgcacatgatgagcgccgccgccatg
ggggccgtcgccttccagaaggggctgggcgcgatccacagcctg
agccatccggtcggcgcggtctacggcacccatcacggcaccacg
aatgcggtggtcatgccgatggtgctggacttcaaccgatccgcc
atcgaggaccggcttgcccgcgccgccgactatcttggcatcaag
ggcggcttcgacggcttccgcgcacgggtgatccagctgcgcagc
gagcttgccatcccccagaacctcacccggctgggggtccagacc
gaacgtctggacgaattgaccgagatggcgctggaggacccgagc
tgcggcggcaacccggtcgagatgacgcgcgagaacacccgcgcg
ctgtttgaaagctgcatgtga
Exemplary alcohol dehydrogenase gene-peg.4723

In one embodiment, the vector comprises a gene encoding an aldehyde dehydrogenase. Aldehyde dehydrogenase (EC 1.2.1.3) is an enzyme that may catalyse the oxidation of aldehydes (e.g. transforms glycolaldehyde into glycolate). In one embodiment, the vector comprises a gene encoding an aldehyde dehydrogenase having at least 70%, at least 75%, at least 80%, at least 85%, at least 90%, at least 95%, at least 96%, at least 97%, at least 98%, at least 99% or 100% sequence identity to SEQ ID NO: 7.

In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 8.

(SEQ ID NO: 7)
MSKTIALISPATGRILVERQTLGIEDARAAVARARAAQPEWAALP
LDERIARIRAGIEALNAMKDAIVPELADQMGRPIRYGGEFGGVNE
RAGHMMKIAAQALAPTVVEDSDHFAREIRREPVGVVFVIAPWNYP
FLTAVNTVVPALVAGNAVILKHASQTLLAGERLAEALHRGGVPAE
VMQNVVLDHQTTEALIAGRSFGFVNFTGSVAGGRAIERAAAGTFT
ATGLELGGKDPGYVRADADLDAAVDGLMDGAMENSGQCCCGIERI
YVHESLFDAFVAKAVDWVNAQKLGNPRDPDTTMGPMAHRRFADLV
RAQVSEAVAQGARPLIDPANFPADDGGAYLAPQVLVDVTHDMRVM
REESFGPVVGIMPVRDDAEAIGLMNDCDYGLTASIWTADADAAAR
IGSRLETGTVYMNRCDYLDPALCWTGCKDTGRGAALSGLGYLAVT
RPKSYHLKKVTK
Exemplary aldehyde dehydrogenase
(SEQ ID NO: 8)
atgagcaagaccatcgccctgatatcgcccgccaccggcaggacc
cttgtcgaaaggcagaccctgggcatcgaggacgcgcgcgccgcc
gttgcccgcgcccgtgccgcccagcccgaatgggcggcgctgccg
ctggacgagcggatcgcccgcatccgcgccgggatcgaggcgctg
aacgccatgaaggatgcgatagtgcccgaactggccgatcagatg
ggccgcccgatccgttacggcggcgaattcggcggcgtcaacgaa
cgcgccggccatatgatgaagatcgcggcccaggctcttgccccg
accgtggtcgaagacagcgatcatttcgcgcgcgagatccggcgc
gagccggtgggcgtggtctttgtcatcgcgccctggaactatccc
ttcctgaccgccgtcaacacggtcgtgcccgccctggtcgccggc
aatgccgtgatcctgaaacatgcgagccagaccctgctggcgggc
gagcgtctggccgaggcgctgcatcggggcggcgttcccgccgag
gtgatgcagaatgtcgtcctggaccaccagacgaccgaggcgctg
atcgccgggcgcagcttcggcttcgtgaacttcacgggctcggtc
gccggagggcgtgcgatcgagcgggccgccgcggggacctttacc
gcgaccgggctggagctgggcggcaaggatcccggctatgtccgc
gccgatgccgatctggatgcggcggtggatgggctgatggacggc
gccatgttcaacagcggccagtgctgctgcgggatcgagcggatc
tatgtccacgaaagcctctttgacgccttcgtggccaaggccgtc
gattgggtgaacgcgcagaagctgggcaatccccgcgacccggat
acgacgatggggccgatggcgcaccggcgctttgccgatctggtc
cgcgcgcaggtctccgaggccgtggcgcagggggcaaggccgctg
atcgacccggcgaactttcccgccgatgatggcggcgcctatctt
gcgccgcaggtgctggtcgacgtgacccacgacatgcgcgtgatg
cgagaggaaagctttggccccgtcgtcggcatcatgccggttcgc
gacgatgccgaggcgatcgggctgatgaacgattgcgactatggg
ctgaccgcctcgatctggaccgccgatgccgatgccgccgcccgg
atcggcagccggcttgaaaccggcaccgtctacatgaaccgctgc
gactatctggacccggcgctgtgctggaccggctgcaaggacacc
ggacgcggcgcggcgttgtcgggccttggctatctggccgtgacg
cggccgaaatcctaccatctgaaaaaggtgacgaaatga
Exemplary aldehyde dehydrogenase gene-peg.4722

In one embodiment, the vector comprises a gene encoding a glyoxylate reductase. Glyoxylate reductase (EC 1.1.1.79) is an enzyme that may catalyse the transformation of glycolate to glyoxylate. In one embodiment, the vector comprises a gene encoding a glyoxylate reductase having at least 70%, at least 75%, at least 80%, at least 85%, at least 90%, at least 95%, at least 96%, at least 97%, at least 98%, at least 99% or 100% sequence identity to SEQ ID NO: 9. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 10.

(SEQ ID NO: 9)
MPAVHATDPTRSRLKVTVTRRLPEAVETRMSELFDVSLNAEDRRM
SREELVAAMRVSDVLVPTITDHIDAAMLAQAGDRLKLIANYGAGV
DHVDVHSARQRGILVSNTPGVVTEDTADVVMALILGVTRRLPEGM
AEMQAGRWQGWSPTAHLGGRLGGRRLGILGMGRIGQAVARRANAF
GMQVHYHNRRRLRPEVEAELQATYWESLDQMLARMDIVSVNAPHT
PSTFHLLNARRLKLLKPSAVVINISRGEVIDENALTRMLRAGEIA
GAGLDVFEHGHEINPRLRELPNVVLLPHMGSATIEGRVEMGEKVI
INIKTFADGHRPPDLVVPSML
Exemplary glyoxylate reductase
(SEQ ID NO: 10)
atgccagccgttcatgcgaccgatccgacccgctcgcgccttaaa
gttaccgtgacccggcgcctgcccgaggcggtcgagacccggatg
tccgagcttttcgacgtttccctgaacgccgaagaccgcaggatg
agtcgcgaggaactggtcgccgccatgcgcgtctcggatgtgctg
gtgccgacgatcaccgatcatatcgacgccgccatgctggcccag
gccggcgaccggctgaagctgatcgccaattacggcgcgggggtg
gaccatgtcgacgtgcattccgcccgccagcgcggcatcctggtc
agcaacaccccgggcgtcgtgaccgaggacacggccgatgtggtc
atggcgctgatcctgggcgttacccggcgcctgcccgagggcatg
gccgagatgcaggccggccgctggcagggctggtcgcccaccgcg
catctgggcgggcggctgggcgggcggcgcctgggcatcctgggc
atgggccgcatcgggcaggcggtggcgcggcgggcgaacgccttc
ggcatgcaggtgcattatcacaaccgccgccgcctgcgccccgag
gtcgaggccgagttgcaggcgacctactgggaaagcctggaccag
atgctggcgcgcatggatatcgtcagcgtgaacgcgccgcatacg
ccctcgaccttccatctgctgaatgcgcggcggttgaagctgctg
aagccctcggccgtggtgatcaacacctcgcgcggcgaggtcatc
gacgagaacgcgctgacccggatgctgcgcgcgggcgagatcgcg
ggtgccggcctggacgttttcgagcatgggcacgagatcaacccc
cgcctgcgcgagctgcccaatgtcgtgttgctgccgcatatgggt
tcggccaccatcgaggggcgggtcgagatgggcgagaaagtcatc
atcaacatcaagaccttcgccgacggccatcggccgccggacctg
gtggtgccctcgatgctgtga
Exemplary glyoxylate reductase gene-peg.1987

### Adipic Acid Pathway

In one embodiment, the vector comprises one or more genes encoding all or part of a pathway for the utilisation of adipic acid. Suitably, the vector comprises a gene encoding a long-chain-fatty-acid-CoA ligase, an acyl-CoA dehydrogenase, an enoyl-CoA hydratase, a 3-hydroxybutyryl-CoA dehydrogenase, and/or a 3-oxoadipyl-CoA thiolase.

In one embodiment, the vector comprises a gene encoding a long-chain-fatty-acid-CoA ligase. Long-chain-fatty-acid-CoA ligase (EC 6.2.1.3) is an enzyme that may ligate Acetyl-CoA to long-chain fatty acids (e.g. adipic acid). In one embodiment, the vector comprises a gene encoding a long-chain-fatty-acid-CoA ligase having at least 70%, at least 75%, at least 80%, at least 85%, at least 90%, at least 95%, at least 96%, at least 97%, at least 98%, at least 99% or 100% sequence identity to SEQ ID NO: 11. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 12.

(SEQ ID NO: 11)
MTPKGRIGGKGMARFASVADRDAVEAEMPYAERQVPHTVYQALTE
TRDRHPQRPAISFQLFSDPKAPARTLTWTELHERVTETANLFRSL
GVGPDDVVAYLLPNCIEAPVVLLAGATAGIVNPINPLLEPDHIAA
ILRETGAKVLVILKSFPKSEVAQKAADAVAQAPNVQTVLEVDLRG
YLTGVKRLLVPLMRPKVTARHHAKVMDFDAAASAQKHNRLTFDEP
AEDRVAAFFHIGGTTGMPKVAQHKQSGMIYNGWLGGTLLFTETDV
LMCPLPMFHVFAAYPVLMSCLMSGAQLVMPTPAGYRGEGVEDNEW
KLIERWQATFLITVPTAIAALMQRPVNADVSSLKTAISGSAPLPI
ELYNRFKAATGVEIAEGYGLTEATCLVSCNPINGLKKVGSVGIPL
PHTHVRILQRRNGGFHECATDEIGEICVANPGVFEGSTYTEADKN
HDLFAESRFLRTGDLGRMDADGYLWITGRAKDLIIRGGHNIDPAE
IEDALLSHPKVAAVAAIGQPDSFAGELPCAYVELIAGAEVGLDEL
MEHARTHIHERAAVPKHVEILPELPKITVGKIFKPDLRKLAIRRV
YDSALAEAGLAAEVGEVVDDRKRGLVAHIRPKGQVDRSAVEQLLG
QYALPWEWVG
Exemplary long-chain-fatty-acid-CoA ligase
(SEQ ID NO: 12)
atgaccccgaaagggagaacgggaggtaaaggaatggcaaggttc
gcaagcgtcgcagaccgcgatgcggtcgaggcggagatgccctat
gcggagcgccaggtgccgcatacggtctatcaggccctgaccgag
acccgtgaccgccatccccagcgccccgcaatcagcttccagctg
ttctcggaccccaaggctccggcccgcacgctgacctggaccgag
ctgcacgagcgggtgaccgagacggcgaacctgttccgcagcctg
ggcgtcgggccggacgatgtcgtcgcctatctgctgcccaactgc
atcgaggcgccggtcgtgctgctggcgggcgcgacggcggggatc
gtgaacccgatcaacccgttgctggagcccgaccacatcgccgcc
attctgcgcgagaccggcgccaaggtgctggtgacgctgaaaagc
tttcctaaatccgaggtcgcccagaaggcggccgatgcggtggcc
caggcgccgaacgtgcagaccgtgctggaggtcgacctgcgcggc
tacctgaccggggtgaaacgcctgctggtgccgctgatgcgaccc
aaggttaccgcgcggcatcacgccaaggtcatggatttcgacgcc
gccgccagcgcgcagaagcacaaccgcctgaccttcgacgaaccg
gccgaggatcgcgtcgccgccttcttccacaccgggggcaccacc
ggcatgcccaaggtcgcccagcacaagcagtcgggcatgatctat
aacggctggctgggcggaacgctgctgttcaccgaaaccgacgtg
ctgatgtgcccgctgccgatgtttcacgtcttcgcggcctatccg
gttctgatgtcctgcctgatgtcgggcgcgcaactggtcatgccg
acgcccgcgggatatcgcggcgaaggcgtgttcgacaacttctgg
aagctgatcgagcgctggcaggcgaccttcctgatcaccgtgccc
accgccatcgccgcgctgatgcagcgccccgtgaatgccgatgtc
tcttcgctcaagaccgcaatctcgggttctgcgcccttgccgatc
gagctttacaaccgcttcaaggcggcgaccggggtcgagatcgcc
gagggctacgggctgaccgaggcgacctgcctcgtgtcctgcaac
ccgatcaacgggctcaagaaggtcggctcggtcggcatccccctg
ccccatacccatgtgcgcatcctgcaacgccggaacggcggtttc
cacgaatgcgccaccgacgagatcggcgagatctgcgtggccaat
cccggcgtcttcgagggctcgacctataccgaggccgacaagaac
cacgatctcttcgccgaaagccgcttcctgcgcaccggagacctc
ggccggatggatgccgacggctacctgtggatcaccgggcgggcc
aaggacctgatcatccgcggcggccacaacatcgaccccgccgag
atcgaggatgcgctgctgtcccatcccaaggtcgccgcggtggcc
gccatcggccaacccgattcctttgccggcgagctgccctgcgcc
tatgtcgagctgatcgcgggcgccgaggtcgggctggacgagttg
atggagcatgcccgcacccatatccacgaacgcgccgcagtgccg
aagcatgtcgagattctgcccgaactgcccaagaccaccgtcggc
aagatcttcaagcccgacctgcgcaagctggcgatccgccgcgtc
tatgactccgccctggccgaggccgggctggcggccgaggtgggc
gaggtggtcgatgaccgcaagcgcggcctggtcgcccacatccgt
cccaagggccaggtcgaccgcagcgccgtcgaacagctgctgggc
caatacgccttgccgtgggagtgggtcggctag
Exemplary long-chain-fatty-acid-CoA ligase
gene-peg.2056

In one embodiment, the vector comprises a gene encoding an acyl-CoA dehydrogenase. Acyl-CoA dehydrogenase (EC 1.3.99.3) is an enzyme that may introduce a trans double-bond between C2 and C3 of a acyl-CoA thioester substrate (e.g. transform adipyl-CoA into 5-Carboxy-2-pentenoyl-CoA). In one embodiment, the vector comprises a gene encoding an acyl-CoA dehydrogenase having at least 70%, at least 75%, at least 80%, at least 85%, at least 90%, at least 95%, at least 96%, at least 97%, at least 98%, at least 99% or 100% sequence identity to SEQ ID NO: 13. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 14.

(SEQ ID NO: 13)
MSLDPETLAQFLETLDRFVRERLIPNEERVADGDAIPPELVQEIR
EMGLFGMSIPEEHGGIGLIMAEEVQAALVLGQASPVFRSLVGINN
GIGSQGIIIDGTPEQKAHYLPQLASGEMIASFALTEPDAGSDAGS
LRCSARLDGDHYVLNGTKRFITNAPHAGLFTVFARTDPDSKSAAG
VTAFLVEAGTPGLHLGPRDRKMGQKGSHTCDVILEDCRVPASAII
GGPDRLGQGFKTAMKVLDRGRLHISAVCVGAAERLIRDSLAYAME
RRQFGEPIAEKQLVQAMLADSRAEAYAARCMIEETARRKDAGLSV
STEAACCKMYASEMVGRVADRAVQIHGGAGYMAEYAVERFYRDVR
LFRIYEGTTQIQQLVIARNMIREASG
Exemplary acyl-CoA dehydrogenase
(SEQ ID NO: 14)
atgagcctcgaccccgaaacgcttgcgcaattcctcgagaccctg
gaccgcttcgtgcgcgaacgcctgatcccgaacgaagagcgggtg
gccgatggcgacgcgatcccgcccgaactggtgcaggagatccgc
gagatgggcctgttcggcatgtcgatccccgaggaacatggcggc
atcggcctgaccatggccgaagaggtgcaggccgcgctggtcctg
ggccaggcctcaccggtgttccgctcgctggtgggcacgaataac
ggcatcggctctcaggggatcatcatcgacggcacgcccgagcag
aaggcgcattacctgccacagctggcctcgggcgagatgatcgcc
tctttcgcgctgaccgagcctgatgccgggtcggacgcgggctcg
ctgcgctgttcggcgcggctggacggcgatcactatgtcctgaac
gggaccaagcggttcatcaccaacgcgccccatgccgggctgttc
accgtcttcgcccgcaccgatcccgacagcaagtcggccgccggc
gtcaccgccttcctggtcgaggccggcacccccggcctgcatctg
gggcccagggaccgcaagatgggccagaagggatcgcatacctgc
gatgtcatcctggaagattgccgcgtcccggcaagtgcgatcatc
ggcggccccgacaggctgggccagggcttcaagaccgcgatgaag
gtgctggaccggggccggctgcacatctcggcggtctgcgtgggc
gcggcggaacggctgatccgcgacagcctggcctatgcgatggag
cgcaggcagttcggcgagcccatcgccgaaaagcagctggtgcag
gcgatgctggccgacagccgcgccgaggcctatgccgcccgctgc
atgatcgaggaaaccgcccggcgcaaggatgccggcctgagcgtc
tcgaccgaggcggcctgctgcaagatgtatgccagcgagatggtc
ggccgcgtggccgaccgcgcggtgcagatccatggaggggcgggc
tacatggccgaatatgcggtcgaacgcttctatcgcgacgtgcgg
ctgttccgcatctatgaaggcacgacccagatccagcagctggtg
atcgcccgcaacatgatccgcgaggccagcggctga
Exemplary acyl-CoA dehydrogenase gene-p.200

In one embodiment, the vector comprises a gene encoding an enoyl-CoA hydratase. Enoyl-CoA hydratase (EC 4.2.1.17) is an enzyme that may hydrate the double bond between the second and third carbons on 2-trans/cis-enoyl-CoA. In one embodiment, the vector comprises a gene encoding an enoyl-CoA hydratase having at least 70%, at least 75%, at least 80%, at least 85%, at least 90%, at least 95%, at least 96%, at least 97%, at least 98%, at least 99% or 100% sequence identity to SEQ ID NO: 15. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 16.

(SEQ ID NO: 15)
MRDISQLNLTHLLFDMDGDGIATVTLNRAAKRNALNAETIEELVA
VFSALPASGARAVVLRAEGPHFCAGLDLVEHGREERSPAEFMRIC
LRWHEAFNKIEYGGIPVIAALKGAVVGGGLELASSVHIRVMDETT
YFGLPEGQRGLFTGGGATIRVPRLIGQARMMDMMLTGRLYSGDEA
VQVGLAQYRVADSEAQAYDLARRVAQNTPLSNFAVCSAISHMQNM
SGLDAAYAEAMVAGIVNTQDAARGRLDSFAQGTAQKIKPGEAG
Exemplary enoyl-CoA hydratase
(SEQ ID NO: 16)
atgcgcgacatttcgcagttgaacctgacccacctgctcttcgac
atggacggggacggcatcgccaccgtcaccctgaaccgcgccgcc
aagcgcaacgccctgaatgccgagacgatcgaggaactggtcgcg
gtcttttccgccctgcccgcctcgggcgcccgtgccgtggtgctg
cgcgccgaggggccgcatttctgcgccgggctggacctggtcgag
cacgggcgcgaggaacgcagccctgccgagttcatgcgcatctgc
ctgcgctggcacgaggcgttcaacaagatcgaatatggcggcatt
ccggtcatcgccgcgctcaagggcgcggtggtgggcggcgggctg
gaactggcctcgtcggtccatatccgggtgatggacgagaccacc
tatttcggcctgcccgaggggcagcgcgggctgttcaccggcggc
ggcgccacgatccgcgtgccccggctgatcggccaggcgcgcatg
atggacatgatgctgaccggccggctgtattccggggacgaggcg
gtgcaggtcgggctggcgcaataccgcgtggccgacagcgaggcg
caggcctacgaccttgcccgccgggtggcgcagaacacacccctg
tcgaatttcgcggtctgctcggcgatctcgcatatgcagaacatg
tcggggctggacgccgcctatgccgaggccatggtcgccggcatc
gtcaacacccaggacgccgccaggggaaggctggacagctttgcc
cagggcacggcgcaaaagatcaagccgggcgaggcaggctga
Exemplary enoyl-CoA hydratase gene-peg.2628

In one embodiment, the vector comprises a gene encoding a 3-hydroxybutyryl-CoA dehydrogenase. 3-hydroxybutyryl-CoA dehydrogenase (EC 1.1.1.157) is an enzyme that may transform 3-hydroxybutyryl-CoA into 3-oxoadipyl-CoA. In one embodiment, the vector comprises a gene encoding a 3-hydroxybutyryl-CoA dehydrogenase having at least 70%, at least 75%, at least 80%, at least 85%, at least 90%, at least 95%, at least 96%, at least 97%, at least 98%, at least 99% or 100% sequence identity to SEQ ID NO: 17. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 18.

(SEQ ID NO: 17)
MAIQSVGVIGAGQMGNGIAHVFALAGYDVLMTDISREALDKAVAQ
IDHNLERQVSRGKVSAEDKAAAMRRITTTMILSDLGKIDLIIEAA
TERETVKQAIFEDLLPHLKPETILTSNISSISITRLASRTDRPER
FMGFHFMNPVPVMQLVELIRGIATNEETYKALVEVVEKIGKISAS
AEDFPAFIVNRILMPMINEAVYTLYEGVGSVKSIDQSMKLGANHP
MGPLELADFIGLDTCLAIMNVLHEGLADTKYRPCPLLVKYVEAGW
LGRKIGRGFYDYSGEEPVPTR
Exemplary 3-hydroxybutyryl-CoA dehydrogenase
(SEQ ID NO: 18)
atggcgattcaatcggtgggcgtgatcggcgccggacagatgggc
aatggcatcgcgcatgtctttgccctggcgggttatgacgtgctc
atgaccgacatctcgcgcgaggcgctggacaaggccgtggcgcag
atcgaccacaacctggaacgccaggtcagccgcggcaaggtctcg
gccgaggacaaggccgcggcgatgcggcgcatcaccaccaccatg
acgctttccgacctgggcaagaccgacctgatcatcgaggccgcc
accgagcgcgagaccgtcaagcaggcgatcttcgaggatctgctg
ccgcatctgaagcccgagaccatcctgacctcgaacacctcgtcg
atctcgatcacccgccttgccagccgcaccgaccggcccgagcgc
ttcatgggcttccacttcatgaacccggttccggtcatgcagctg
gtcgagctgatccgcggcatcgccaccaacgaggagacctacaaa
gccctggtcgaagtggtcgaaaagatcggcaagacctcggccagc
gccgaggatttcccggccttcatcgtcaaccgcatcctgatgccg
atgatcaacgaggcggtctatacgctttacgagggcgtcggctcg
gtcaagtccatcgaccagtcgatgaagctgggcgccaaccacccg
atggggccgctggaactggcggatttcatcggcctcgacacctgc
ctggcgatcatgaacgtgctgcacgaggggctggcggacacgaaa
taccggccctgcccgctcttggtgaaatatgtcgaggcaggctgg
ctgggccgcaagaccgggcgtgggttctacgactattcgggcgag
gagccggtgccgacgcgatag
Exemplary 3-hydroxybutyryl-CoA dehydrogenase
gene-peg.2620

In one embodiment, the vector comprises a gene encoding a 3-oxoadipyl-CoA thiolase. 3-oxoadipyl-CoA thiolase (EC 2.3.1.174) is an enzyme that may transform 3-oxoadipyl-CoA into succinyl-CoA. In one embodiment, the vector comprises a gene encoding a 3-oxoadipyl-CoA thiolase having at least 70%, at least 75%, at least 80%, at least 85%, at least 90%, at least 95%, at least 96%, at least 97%, at least 98%, at least 99% or 100% sequence identity to SEQ ID NO: 19. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 20.

(SEQ ID NO: 19)
MTEVFICDYIRIPIGREGGALSSVRADDLGAIPLRALMARHAGLD
WQAVDDVIYGCANQAGEDNRNVARMSALLAGLPVEVPGITTINRL
CGSGMDAVLVAARQIAAGEAELMIAGGVESMSRAPFVLPKAESAF
SRHAEIHDTTIGWRFVNPAMHAAYGTDSMPQTGQNVADDYGISRE
AQDAMALASQQKAAAAIASGRLAAEIAPITIPQRKGEPIVVDTDE
HPRATTPEALAKLRPLFPNGSVTAGNASGVNDGAAALILASEAAA
RKHGLIPIARVLGGATAGVPPRIMGIGPAPASQKLMDRLGLIPAD
EDVIELNEAFAAQGLATLRQLGIADDDPRVNPNGGAIALGHPLGM
SGARITGTAALELALTGGKRSLSTMCIGVGQGIAVALERV
Exemplary 3-oxoadipyl-CoA thiolase
(SEQ ID NO: 20)
atgaccgaagtctttatctgcgactatatccgcacgcccatcggc
cggtttggcggcgcgctgtcctcggtgcgcgccgacgatctgggt
gccatccccttgcgcgcgctgatggcacgccatgccggcctcgac
tggcaggcggtggacgacgtgatctatggctgcgccaaccaggcc
ggcgaggacaaccgcaacgtcgcccgcatgtcagcgctgctggcc
gggctgccggtcgaggtgccaggcacgacgatcaaccggctctgc
ggctcgggcatggacgcggtgctggtcgccgcgcgccagatcgcg
gcgggcgaggccgagctgatgatcgccggcggcgtcgagtcgatg
tcgcgcgcgcccttcgtgctgcccaaggccgaaagcgccttcagc
cgccatgccgagatccacgacaccaccatcggctggcgcttcgtg
aacccggcgatgcatgcggcctatggcaccgactcgatgccgcag
accggccagaacgtcgccgacgactacggcatctcgcgcgaggcg
caggacgcgatggcgctggcctcgcagcagaaggccgcggcggcc
atcgccagcggccggctcgcagccgagatcgcgccgatcacgatc
ccgcagcgcaagggcgagcccatcgtggtcgataccgacgagcat
ccccgcgccaccacgcccgaggcgctggccaagctgcgaccgctg
ttcccgaacggctcggtcacggccggcaatgcctcgggggtgaac
gacggggcggcggcgctgatcctggcctcggaggcggcggcgagg
aagcatggactgacgcccatcgcccgcgtgctgggcggggcgacg
gcgggcgtgccgccgcgcatcatgggcatcggcccggcgccggcc
tcgcaaaaactgatggatcggctgggcctgaccccggccgatttc
gacgtgatcgagctgaatgaggccttcgccgcccagggcctcgcc
acgctgcgccagctgggcatcgccgacgacgacccgcgcgtgaac
ccgaacggcggcgccatcgcgctggggcatccgctgggcatgtcg
ggcgcccggatcaccggcaccgcggcgctggaactggcgctgacg
ggtggcaaacgctcgctctcgaccatgtgcatcggggtgggacaa
ggcatcgccgtggcgctggaaagggtctga
Exemplary 3-oxoadipyl-CoA thiolase gene-peg.5206

### 6-Hydroxycaproic Acid Pathway

In one embodiment, the vector comprises one or more genes encoding all or part of a pathway for the utilisation of 6-hydroxycaproic acid. Suitably, the vector comprises a gene encoding an alcohol dehydrogenase and/or an aldehyde dehydrogenase.

In one embodiment, the vector comprises a gene encoding an alcohol dehydrogenase. Alcohol dehydrogenase may transform 6-hydroxycaproic acid into 6-oxocaproic acid. In one embodiment, the vector comprises a gene encoding an alcohol dehydrogenase having at least 70%, at least 75%, at least 80%, at least 85%, at least 90%, at least 95%, at least 96%, at least 97%, at least 98%, at least 99% or 100% sequence identity to SEQ ID NO: 21. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 22.

(SEQ ID NO: 21)
MAKTMKAAVVREFGKPLTIDEVPVPEPGPGMIQVRIQASGVCHTD
LHAAEGDWPVKPNPPFIPGHEGVGFVSAVGAGVKHVKEGDRVGVP
WLYTACGHCRHCLGGWEILCESQLNIGYSVNGGFADYVVADPNYV
GHLPKNVDFLDIAPVLCAGVTVYKGLKVTDTKPGDWVVISGIGGL
GHMAVQYAKAMGMNVAAVDIDDEKLALARKLGATVTVNAATEPDP
AAAIRKQTDGGAQGVLVTAVGRKAFEQAIGMVARGGTVALNGLPP
GDFPLDIFGMVLNGITVRGSIVGIRLDLQESLDFAGDGKVKATVH
KAKLEDINNIFGQMHKGQIEGRMVLDMAG
Exemplary alcohol dehydrogenase
(SEQ ID NO: 22)
atggccaaaaccatgaaagccgccgtcgtgcgcgaattcggcaag
cccctgaccatcgacgaggtgccggtccccgaacccggccccggc
atgatccaggtcaggatccaggcctcgggcgtctgccataccgac
ctgcacgcggccgagggcgactggccggtcaagccgaacccgccc
ttcattcccggccatgaaggcgtgggcttcgtctcggccgtgggc
gccggggtcaagcacgtcaaggagggcgaccgcgtcggcgtgccc
tggctctacaccgcctgcggccattgccggcattgcctgggcggc
tgggaaacgctgtgcgaaagccagctcaacaccggctattcggtg
aacggtggctttgccgattacgtcgtggccgatccgaactatgtc
ggccacctgccgaagaacgtcgatttcctggacatcgccccggtg
ctctgcgcgggcgtcacggtctacaaggggctgaaggtcacggat
accaagcccggcgactgggtggtgatctcgggcatcggcgggctg
gggcatatggcggtgcaatatgccaaggccatgggcatgaacgtc
gccgccgtggacatcgacgacgaaaagctggcactggcccgcaag
ctgggcgccacggtgacggtgaacgccgcgaccgaacccgacccg
gcggcggcgatcaggaaacagaccgatggcggcgcacagggcgtg
ctggtgacggcggtcggccgcaaggccttcgagcaggccatcggc
atggtcgcacgcggcggcacggtggcgctgaacggcctgcccccg
ggcgacttcccgctggacatcttcggcatggtgctgaacggcatc
accgtgcgcggctcgatcgtgggcacgcggctggacctgcaggaa
tcgctggatttcgccggcgacggcaaggtcaaggccaccgtgcac
aaggcaaagctcgaagacatcaacaacatcttcggccagatgcac
aaaggccagatcgaaggccgcatggtgctggacatggcgggctga
Exemplary alcohol dehydrogenase gene-peg.2426

In one embodiment, the vector comprises a gene encoding an aldehyde dehydrogenase. Aldehyde dehydrogenase may transform 6-oxocaproic acid into adipic acid. In one embodiment, the vector comprises a gene encoding an aldehyde dehydrogenase having at least 70%, at least 75%, at least 80%, at least 85%, at least 90%, at least 95%, at least 96%, at least 97%, at least 98%, at least 99% or 100% sequence identity to SEQ ID NO: 23. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 24.

(SEQ ID NO: 23)
MPNDQTHPFRGVNALPFEERYDNFIGGEWVAPVSGRYFINITPIT
GAEIGQIARSEAGDIELALDAAHAAKEKWGAISPAERANIMLKIA
DRMERNLELLATAETWDNGKPIRETMAADLPLAIDHERYFAGVLR
AQEGSISQIDDDTVAYHFHEPLGVVGQIIPWNFPLLMACWKLAPA
IAAGNCVVLKPAEQTPAGIMVWANLIGDLLPPGVLNIVNGFGLEA
GKPLASSNRIAKIAFTGETTIGRLIMQYASENLIPVTLELGGKSP
NIFFADVAREDDDFFDKALEGFTMFALNQGEVCTCPSRVLIQESI
YDKFMERAVQRVQAIKQGDPRESDTMIGAQASSEQKEKILSYLDI
GKKEGAEVLTGGKAADLGGELSGGYYIEPTIFRGNNKMRIFQEEI
FGPVVSVTTFKDQAEALEIANDTLYGLGAGVWSRDANTCYRMGRG
IKAGRVWINCYHAYPAHAAFGGYKQSGIGRETHKMMLDHYQQTKN
MLVSYSPKKLGFF
Exemplary aldehyde dehydrogenase
(SEQ ID NO: 24)
atgccgaacgaccagacgcatcccttccggggcgtgaacgcgctg
cccttcgaggaacgctacgacaatttcatcggcggcgaatgggtg
gcgcccgtctcgggcaggtatttcaccaacaccaccccgatcacc
ggcgccgagatcgggcagatcgcgcgctcggaggccggcgacatc
gagctggcgctggacgccgcccatgccgccaaggagaaatggggc
gccaccagcccggccgagcgcgccaacatcatgctgaagatcgcc
gaccggatggagcggaacctggagctgctcgccaccgccgagacc
tgggacaacggcaagccgatccgcgagaccatggccgccgacctg
ccgctggccatcgaccatttccgctatttcgccggcgtgctgcgg
gcgcaggagggctcgatcagccagatcgacgacgacaccgtcgcc
tatcacttccacgagccgctgggcgtggtgggccagatcatcccc
tggaactttccgctgctgatggcctgctggaagctggcccccgcg
atcgccgccggcaactgcgtggtgctgaaaccggccgagcagacc
ccggccggcatcatggtctgggccaacctgatcggcgacctgctg
ccgccgggcgtgctgaacatcgtcaacggcttcgggctggaagcc
ggcaagccgctggcatcctcgaaccgcatcgccaagatcgccttc
accggcgagacgacgaccggccggctgatcatgcaatatgccagc
gagaacctgatcccggtgacgctggaactgggcggcaagtctccg
aacatcttctttgccgatgtcgcgcgcgaggatgacgatttcttc
gacaaggcgctggaaggctttaccatgttcgcgctgaaccagggc
gaggtctgcacctgcccgtcgcgcgtgctgatccaggaatcgatc
tatgacaagttcatggaacgcgcggtccagcgcgtacaggcgatc
aagcagggcgatccgcgcgaaagcgacaccatgatcggcgcgcag
gcgtcgagcgagcagaaggaaaagatcctcagctatctcgacatc
ggcaagaaggaaggcgccgaggtgctgaccggcggcaaggcggcc
gacctgggcggcgagctttcgggcggctactacatcgagccgacg
atctttcgcggcaacaacaagatgcggatcttccaggaggaaatc
ttcggcccggtggtctcggtcaccaccttcaaggaccaggccgag
gcgctggagatcgccaacgacacgctttacggccttggcgccggc
gtgtggtcgcgcgatgccaatacctgctatcgcatgggccgcggc
atcaaggccggccgggtctggaccaactgctaccacgcctatccg
gcccatgcggccttcggcggctacaagcagtcgggcatcgggcgc
gagacgcacaagatgatgctggaccactatcagcagaccaagaac
atgctggtcagctattcgcccaagaagctgggcttcttctga
Exemplary aldehyde dehydrogenase gene-peg.2425

### 3-Hydroxybutyric Acid Pathway

In one embodiment, the vector comprises one or more genes encoding all or part of a pathway for the utilisation of 3-hydroxybutyric acid. Suitably, the vector comprises a gene encoding an Acyl-CoA synthetase, and/or a 3-hydroxybutyrate dehydrogenase.

In one embodiment, the vector comprises a gene encoding an Acyl-CoA synthetase. Acyl-CoA synthetase is an enzyme that may catalyse the activation of free fatty acids) to CoA esters (e.g. transform 3-hydroxybutyric acid to 3-hydroxybutyril-CoA and/or transform 3-hydroxyvaleric acid into 3-hydroxyvaleryl-CoA). In one embodiment, the vector comprises a gene encoding an Acyl-CoA synthetase having at least 70%, at least 75%, at least 80%, at least 85%, at least 90%, at least 95%, at least 96%, at least 97%, at least 98%, at least 99% or 100% sequence identity to SEQ ID NO: 25. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 26.

(SEQ ID NO: 25)
MSATTLSRSRPGYEQAVAQFRIEDAIAGLRGDLETGLNACVECCD
RHCGADRVALRCLSADEALVEYTFEDLRALSARAANLMRDKGIRP
GDVVAGLLPRIVELVATVLGAWRLGAVYQPLFTAFGPKAIEHRLK
TSGAKLVVTNAAQRPKLDEVEDCPLVATLRGDGPLAPGDVDFREA
LAMASDQFEPVMRRGEDLFMMMSTSGTTGLPKGVPVPLRALLAFG
AYMRDAIDLRETDVFWNIADPGWAYGLYYALTGPLLLGQPTILYE
GGFTAETTYRIIERMGVTSLAGSPTAYRLLIAAGPEAAGAVKGRL
RVVSSAGEPLNPEIIRWFGEHLAVPIHDHYGQTEMGMCVNNHHGL
EHPVRPGSAGLAMPGYRIVVLDDDGNELGPNQPGVLAIDMKRSPL
MWFSGYLNQATPALAGGYYRTGDSVEFEPDGSISFIGRSDDVITS
SGYRIGPFDVESALIEHPAVVEAAVVGVPDPERTEIVKAFVVLAK
GVEGTEALREELAQHVKKRLSAHAYPRMIDFVADLPKTPSGKIQR
FVLRKAEVEKLARE
Exemplary Acyl-CoA synthetase
(SEQ ID NO: 26)
atgtctgcgacgaccctgtcccggtcgcggcccggctatgagcag
gccgtggcgcaattccgcatcgaggacgccatcgccgggctgcgc
ggcgacctggagaccgggctgaacgcctgcgtcgaatgctgcgac
cgccattgcggcgcggaccgggtggcgctgcgctgcctgtcggcg
gacgaggcgctggtggaatacacattcgaggatctgcgcgcgctg
tcggcgcgggcggcgaacctgatgcgcgacaagggcatccggccg
ggcgacgtggtggcggggctgttgccgcgcacggtcgagctggtc
gcgacggtgctgggcgcctggcggctgggcgcggtctatcagccg
ctgttcaccgcctttggtcccaaggccatcgagcaccggctgaag
accagcggcgccaagctggtggtgacgaatgccgcgcagcggccc
aagctggacgaggtcgaggactgcccgctggtcgccaccctgcgc
ggcgacgggccgctggcgccgggcgatgtcgatttccgcgaggcg
ctggccatggcctcggaccagttcgagccggtgatgcggcgcggc
gaggatctgttcatgatgatgtccacctcgggcaccacagggctg
cccaagggggtgccggtgccgttgcgggcgctcttggcattcggc
gcctatatgcgcgacgccatcgacctgcgcgagacggacgtgttc
tggaacatcgccgatccgggctgggcttacgggctttactatgcg
ctgaccgggcccctgctgctgggccagccgacgatcctctacgaa
ggcggctttaccgccgagacgacctatcgcatcatcgagcgcatg
ggggtgacgagccttgccggctcgcctaccgcataccggctgctg
atcgcggcggggccggaggcggccggggcggtcaaggggcggctg
cgggtggtcagttccgccggcgagcccctgaaccccgagatcatc
cgctggttcggcgagcatctggcggtgccgatccacgaccattac
ggccagaccgagatgggcatgtgcgtgaacaaccatcacgggctg
gagcatccggtgcgtccgggctcggccgggctggcgatgccgggc
tatcgcatcgtggtgctggacgatgacggcaacgaactgggaccg
aaccagccgggcgtgctggccatcgacatgaagcgttcgccgctg
atgtggttttccggctatctgaaccaggcgacgccggcgctggcg
gggggctattaccgcaccggcgacagcgtggagttcgagccggac
gggtcgatcagctttatcgggcggtcggacgacgtgatcacgtcc
tcgggctatcgcatcgggcccttcgacgtggaaagcgcgctgatc
gagcatcctgcggtggtcgaggcggcggtggtcggcgtgccggat
cccgagcggaccgagatcgtgaaagccttcgtcgtgctggccaag
ggggtcgaggggaccgaggcgctgcgcgaggaactggcgcagcat
gtcaagaaacgcttgtcggcccatgcctatccccggatgatcgat
ttcgtcgccgacctgcccaagacgccgagcggcaagatccagcgc
tttgtcctgcgcaaggcggaagtcgaaaaactggcccgggagtaa
Exemplary Acyl-CoA synthetase gene-peg.2994

In one embodiment, the vector comprises a gene encoding a 3-hydroxybutyrate dehydrogenase. 3-hydroxybutyrate dehydrogenase (EC 1.1.1.30) is an enzyme that may transform 3-hydroxybutyrate into acetoacetate and/or 3-hydroxyvalerate into 3-oxopentanoic acid. In one embodiment, the vector comprises a gene encoding a 3-hydroxybutyrate dehydrogenase having at least 70%, at least 75%, at least 80%, at least 85%, at least 90%, at least 95%, at least 96%, at least 97%, at least 98%, at least 99% or 100% sequence identity to SEQ ID NO: 27. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 28.

(SEQ ID NO: 27)
MFEKFLSGKTAVVTGSNSGIGLGIAHELARAGADLVLNSFTDMPE
DHALAESLAAEHGVEVRYVQADMSKGADCRALIEKAGACDILVNN
AGIQHVAPIPDFPGEKWDAIIAINLSSAFHTTAAALPLMRKAGWG
RVINIASAHGLTASEYKSAYVAAKHGIVGLTKVTALETAKEPITC
NAICPGYVLIPIVEKQIPDQMKTHNMSREDVIAKVMLQRQPSGQF
ATVEQMGGTAVFLCSPAAEQITGITTISVDGGWTAL
Exemplary 3-hydroxybutyrate dehydrogenase
(SEQ ID NO: 28)
atgttcgagaaattcctcagcggcaagacggcggtggtgacgggc
tccaattcggggatcgggctcgggatcgcgcatgaactggcgcgc
gcgggcgctgatctcgtgctgaacagctttaccgacatgcccgag
gaccacgcccttgccgaaagccttgccgccgagcatggcgtcgag
gtgcgctatgtccaggccgacatgtccaagggcgccgactgccgc
gccctgatcgaaaaggccggcgcctgcgacatcctggtgaacaat
gccggcatccagcatgtcgcaccgatcccggatttcccgggcgag
aaatgggatgcgatcatcgccatcaacctgagttccgcctttcac
accacggcggcggcgctgcccctgatgcgcaaggcaggctggggg
cgggtgatcaacatcgcctcggcgcacgggctgacggccagcgaa
tacaaatcggcctatgtcgcggccaagcacggcatcgtcggcctg
accaaggtgacggcgctggagaccgcgaaggagccgatcacctgc
aacgccatctgccccggctatgtgctgaccccgatcgtggaaaag
cagatccccgaccagatgaagacccacaacatgagccgcgaggac
gtgatcgccaaggtcatgctgcagcgccagccctcggggcaattc
gccacggtcgagcagatgggcggcacggcggtcttcctgtgctcg
ccggcggcggagcagatcaccggcacgaccatctcggtggacggg
gggtggacggcgctttag
Exemplary 3-hydroxybutyrate dehydrogenase
gene-peg.960

### 3-Hydroxyvaleric Acid Pathway

In one embodiment, the vector comprises one or more genes encoding all or part of a pathway for the utilisation of 3-hydroxyvaleric acid. Suitably, the vector comprises a gene encoding an Acyl-CoA synthetase, a 3-hydroxybutyrate dehydrogenase, and/or a 3-ketoacyl-CoA thiolase.

In one embodiment, the vector comprises a gene encoding a 3-ketoacyl-CoA thiolase. 3-ketoacyl-CoA thiolase (EC 2.3.1.16) is an enzyme that may break down 3-oxopentanoyl-CoA into acetyl-CoA and propionyl-CoA. In one embodiment, the vector comprises a gene encoding a 3-ketoacyl-CoA thiolase having at least 70%, at least 75%, at least 80%, at least 85%, at least 90%, at least 95%, at least 96%, at least 97%, at least 98%, at least 99% or 100% sequence identity to SEQ ID NO: 29. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 30.

(SEQ ID NO: 29)
MSENEIVILSGARTAIGTFGGSLAGVPPIQLAATVTRAAIERAGI
GPERIGTVVFGHVLNTEPRDMYLSRVAMLDAGVPDTIPAMNVNRL
CGSGAQAIVSATQALILGDADFAVAGGAESMSRAPYAVPAARFGA
KMGDVQMLDMMVGALTCPMGIGHMGVTAENVAREHDISRQAQDEF
ALESQKRAAAAIAEGRFKEQIVPIEIKTRKGMVAFDTDEHPKATD
LEKLAGLKAVFQKDGTVTAGNASGINDGAAALVLARADAARAAGA
KPLFRVLGYAVAGVRPEVMGIGPVPAVEALLKSTGLKIGEFDVIE
SNEAFAAQALAVNKGLGLDPAKVNPNGGAIALGHPVGATGALVTV
KAMYELMRTGGSKGLIIMCIGGGQGIALAIERI
Exemplary 3-ketoacyl-CoA thiolase
(SEQ ID NO: 30)
atgtcagagaacgaaatcgtcatcctgtcgggggcgcgcacggcc
atcggcaccttcggcggcagcctcgcgggggtgcctccgatccag
cttgccgcgacggtcacccgtgcggcgatcgagcgcgcggggatc
ggccccgagcgcatcggcaccgtggtcttcggccatgtgctgaac
accgagccgcgcgacatgtatctgtcgcgcgtggcgatgctggac
gccggcgtgcccgataccacgccggcgatgaacgtgaacaggctc
tgcggttcgggggcgcaggccatcgtctcggccacgcaggcgctg
atactgggggatgccgactttgccgtggcgggcggtgcggaatcg
atgagccgcgcgccctatgccgtgccggcggcccggttcggcgcc
aagatgggcgacgtgcagatgctcgacatgatggtcggcgcgctg
acctgcccgatgggcaccggccatatgggcgtgacggccgagaat
gttgcgcgcgaacatgacatctcacgccaggcgcaggacgaattc
gcgctggaaagccagaagcgcgctgcggccgccatcgccgagggc
cgcttcaaggagcagatcgttcccatcgagatcaagacccgcaag
ggcatggtggccttcgataccgacgagcacccgaaagcgaccgat
ctggagaagctggcgggactgaaggcggttttccagaaggatggc
acggtcaccgccggcaacgcctcgggcatcaacgacggcgcggcg
gctctggtgctggcgcgcgcggacgcggccagggcggcgggcgcc
aagccgctgttccgggtgctcggctatgccgtggcgggcgtgcgt
cccgaggtcatgggcatcggcccggtcccggcggtcgaggcgttg
ctcaagagcaccggcctgaagatcggcgagttcgacgtgatcgag
tcgaacgaggctttcgccgcgcaggctttggcggtgaacaagggg
ctcggcctcgatccggcgaaggtgaacccgaacggcggcgccatc
gccttgggccatccggtcggcgcgaccggcgccctcgtcacggtc
aaggcgatgtatgaattgatgcggaccggcggcagcaaggggctg
atcaccatgtgcatcggcggcggccagggcatcgcgctggctatc
gagcgcatctga
Exemplary 3-ketoacyl-CoA thiolase gene-peg.2743

### 1,4-Butanediol Pathway

In one embodiment, the vector comprises one or more genes encoding all or part of a pathway for the utilisation of 1,4-butanediol. Suitably, the vector comprises a gene encoding a methanol dehydrogenase, an aldehyde dehydrogenase, an alcohol dehydrogenase, and/or a succinate-semialdehyde dehydrogenase.

In one embodiment, the vector comprises a gene encoding a methanol dehydrogenase. A methanol dehydrogenase (EC 1.1.2.7) is an enzyme that may transform 1,4-butanediol to 4-hydroxybutyraldehyde. In one embodiment, the vector comprises a gene encoding a methanol dehydrogenase having at least 70%, at least 75%, at least 80%, at least 85%, at least 90%, at least 95%, at least 96%, at least 97%, at least 98%, at least 99% or 100% sequence identity to SEQ ID NO: 31. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 32.

(SEQ ID NO: 31)
MKNLMNGACLALLMSGTAALANDSVLAEIAKPQQWAIQMGDYANT
RYSTLDQINKDNVKDLRVAWTFSTGVLRGHEGSPLVIGDVMYVHT
PFPNRVFALDLNDNGKILWRYEPQQDPNVIAVMCCDTVYRGLSYA
DGMILLGQADTTVVALDATSGEVKWSTKIGDPGIGETLTATVVPV
KDKVLVGISGGEYGVRGRMTALNLIDGSEAWKAWSTGPDEELLVD
PETITHLGKPIGADSSLNSWEGDQWQIGGGTIWGWFSYDPDLNLV
YYGIGNPSTWNPSQRPGDNKWSMTIMARDADIGMAKWFYQMTPHD
EWDYDGVNEMILINQTVDGQERKLLTHFDRNGLAYTMDRETGELL
VAEKYDPVVNWTTGVDMDPNSETYGRPAVVAEYSTAQNGEDENTT
GVCPAALGTKDQQPAAFSPKTNLFYVPINHVCMDYEPFRVAYTAG
QPYVGAILSMYPAPNSHGGMGNFIAWDNITGEIKWSVPEQFSVWS
GALATAGDVVFYGTLEGYLKAVDAQTGEELYKFKTPSGIIGNVMT
YEHGGKQYVGILSGVGGWAGIGLAAGLINPNDGLGAVGGYASLSQ
YTELGGQLIVFELPG
Exemplary methanol dehydrogenase
(SEQ ID NO: 32)
atgaaaaacctaatgaatggcgcctgccttgcgctgctcatgtcc
ggcactgcggcgctggccaatgacagcgtgctggccgagatcgcc
aagccccagcaatgggcgatccagatgggcgattacgcgaacacg
cgatattcgaccctggaccagatcaacaaggacaacgtcaaggat
ctgcgcgtcgcctggaccttctcgaccggggtgctgcgcggacac
gaaggctcgccgctggtgatcggcgatgtcatgtatgtccacacc
ccctttccgaaccgggtgtttgcgctggacctgaacgacaacggc
aagatcctgtggcgctatgaaccgcagcaggatccgaacgtcatt
gcggtgatgtgctgcgacacggtctatcgcggcctttcctatgcg
gacggcatgatcctgctgggccaggccgacaccacggtcgtggcc
ctggacgccacgagcggcgaggtgaaatggtcgaccaagatcggc
gacccgggcatcggcgagacgctgaccgccaccgtcgttccggtc
aaggacaaggtcctggtcggcatctcgggcggcgaatacggcgtg
cgcggccggatgaccgcgctgaacctcaccgatggcagcgaggcc
tggaaagcctggtccaccggcccggacgaggagttgctggtcgat
cccgaaacgaccacgcatctgggcaagcccatcggcgccgacagc
tcgctcaacagctgggaaggcgatcagtggcagatcggcggcggc
acgatctggggctggttctcgtatgacccggacctgaacctggtc
tattacggcaccggcaacccctcgacctggaacccctcgcagcgg
ccgggcgacaacaagtggtcgatgacgatcatggcgcgggatgcc
gataccggcatggccaagtggttctatcagatgacgccccatgac
gaatgggactatgacggcgtcaacgagatgatcctgaccaaccag
accgtcgacgggcaagagcgcaagctgctgacccatttcgaccgc
aacggcctggcctatacgatggatcgcgagaccggcgagttgctg
gtggccgagaaatacgatccggtggtgaactggaccaccggcgtc
gacatggacccgaattcggaaacctatggccgtccggccgtggtg
gccgaatactcgaccgcccagaacggcgaggacgagaacaccacc
ggcgtctgccctgcggcactgggcaccaaggaccagcagccggcg
gccttctcgcccaagaccaacctgttctatgtgccgacgaaccac
gtctgcatggattacgagccgttccgggtggcctataccgccggc
cagccctatgtcggcgccacgctgtccatgtatcccgcgccgaac
agccatggcggcatgggcaacttcatcgcctgggacaacaccacg
ggcgagatcaagtggtccgttcccgagcagttctcggtctggtcg
ggcgctttggctacggcgggcgacgtggtcttctacggcacgttg
gagggctatctgaaggccgtcgatgcccagacgggcgaggagctt
tacaagttcaagaccccctcgggcatcatcggcaacgtgatgacc
tacgaacatggcggcaagcagtatgtcggcatcctgtcgggtgtc
ggcggctgggccgggatcggccttgccgcgggcctgaccaatccc
aacgatgggcttggcgcggtgggcggctatgcctcgctgtcgcaa
tataccgagctgggcggccagctgaccgtgttcgaactgccgggc
taa
Exemplary methanol dehydrogenase gene-peg.20

In one embodiment, the vector comprises a gene encoding a methanol dehydrogenase having at least 70%, at least 75%, at least 80%, at least 85%, at least 90%, at least 95%, at least 96%, at least 97%, at least 98%, at least 99% or 100% sequence identity to SEQ ID NO: 33. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 34.

(SEQ ID NO: 33)
MNRNTPKARGASSLAMAVAMGLAVLITAPATANDQLVELAKDPAN
WVMIGRDYNAQNYSEMIDINKENVKQLRPAWSFSTGVLHGHEGTP
LVVGDRMFIHTPFPNTTFALDLNEPGKILWQNKPKQNPTARTVAC
CDVVNRGLAYWPGDDQVKPLIFRTQLDGHIVAMDAETGETRWIME
NSDIKVGSILTIAPYVIKDLVLVGSSGAELGVRGYVTAYDVKSGE
MRWRAFATGPDEELLLAEDENAPNPHYGQKNLGLETWEGDAWKIG
GGTNWGWYAYDPEVDLFYYGSGNPAPWNETMRPGDNKWIMAIWGR
EATTGEAKFAYQKTPHDEWDYAGVNVMMLSEQEDKQGQMRKLLTH
PDRNGIVYTLDRINGDLISADKMDDTVNWVKEVQLDTGLPVRDPE
FGTRMDHKARDICPSAMGYHNQGHDSYDPERKVEMLGINHICMDW
EPFMLPYRAGQFFVGATLTMYPGPKGDRQNALGLGQIKAYDAISG
EMKWEKMERFSVWGGTMATAGGLIFYGTLDGFIKARDSDIGDLLW
KFKLPSGVIGHPMTYKHDGRQYVAIMYGVGGWPGVGLVFDLADPT
AGLGSVGAFKRLQEFTQMGGGVMVFSLDGESPYSDPNVGEYAPGE
PT
Exemplary methanol dehydrogenase
(SEQ ID NO: 34)
atgaacaggaacaccccgaaggcaaggggcgcgtcgagccttgcg
atggccgtggccatgggccttgccgtcctgacgaccgcgccggcg
accgccaatgaccagttggtcgaactggccaaggaccccgcgaac
tgggtcatgaccgggcgcgactacaacgcgcagaactattccgag
atgaccgacatcaacaaggagaacgtcaagcagctgcggcccgcc
tggtcgttctcgaccggggtgctgcatgggcacgaaggtacgccg
ctggtcgtcggcgaccggatgttcattcacacgcccttcccgaac
accaccttcgcgctggacctgaacgagccgggcaagatcctgtgg
cagaacaagcccaagcagaaccccacggcccggactgtggcctgc
tgcgacgtggtcaaccgcggcctggcctattggccgggcgacgat
caggtcaagccgctgatcttccgcacccagctcgacggccatatc
gtcgccatggatgccgagaccggcgagacgcgctggatcatggag
aactcggacatcaaggtcggctcgaccctgaccatcgcgccctat
gtcatcaaggacctggtgctggtcggctcctcgggcgccgagctg
ggcgtgcgcggctatgtcaccgcctatgacgtgaaatcgggcgag
atgcgctggcgcgcctttgccaccggcccagacgaggaattgctg
ctggccgaggacttcaacgccccgaacccgcattacggccagaag
aacctgggcctggagacctgggagggcgacgcctggaagatcggc
ggcggcaccaactggggctggtatgcctatgaccccgaggtggac
ctgttctactacggctcgggcaaccccgcgccctggaacgagacc
atgcgtccgggcgacaacaagtggaccatggcgatctggggccgc
gaggccaccaccggcgaggcgaaattcgcctatcagaagacgccg
catgacgaatgggactatgccggcgtcaacgtgatgatgctgtcg
gaacaggaggacaagcagggccagatgcgcaagcttctgacccac
ccggaccgcaacggcatcgtctacacgctggaccgcaccaatggc
gacctgatctcggccgacaagatggacgacacggtcaactgggtg
aaggaggtgcagctggataccggcctgccggtgcgcgacccggaa
ttcggcacgcgcatggaccacaaggcccgcgacatctgtccctcg
gcgatgggctatcacaaccagggccatgacagctacgaccccgag
cgcaaggtgttcatgctgggcatcaaccacatctgcatggattgg
gagcccttcatgctgccctatcgcgccggccagttcttcgtcggc
gccacgctgaccatgtatccgggccccaagggcgaccgccagaac
gcgctggggctgggccagatcaaggcctacgacgccatctcgggc
gagatgaaatgggaaaagatggagcgcttctcggtctggggcggc
accatggccaccgcgggcgggctgaccttttacggcacgctggac
ggcttcatcaaggcccgcgacagcgacaccggcgacctgctgtgg
aagttcaagctgccctcgggcgtgatcggccatccgatgacctac
aagcatgacggccggcaatatgtcgcgatcatgtatggagtcggc
ggctggccgggcgtgggtctggtcttcgacctggccgacccgacc
gccggtctgggctcggtgggcgcgttcaagcggctgcaggagttc
acccagatgggcggcggcgtgatggtcttctcgcttgacggcgag
agcccctattccgacccgaacgtgggcgaatacgcgccgggtgag
cccacctga
Exemplary methanol dehydrogenase gene-peg.3083

Aldehyde dehydrogenase may convert 4-hydroxybutyraldehyde to 4-hydroxybutyrate. In one embodiment, the vector comprises a gene encoding an aldehyde dehydrogenase having at least 70%, at least 75%, at least 80%, at least 85%, at least 90%, at least 95%, at least 96%, at least 97%, at least 98%, at least 99% or 100% sequence identity to SEQ ID NO: 35. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 36.

(SEQ ID NO: 35)
MTRSFDPDILDLPRGHFIAGEHVADRGRLAMHRPSDGAAFGESPV
ADADMVDRAVAAGRAALAASGWGCGVPRDRTRALLKWADLIEAEA
ETLARFEAACSTRPVAQLPVGDIAVTAEQIRFFAEMADKEGSDLV
PTRDASLGMTVDEPYGVVGAITPWNFPLSMAGWKLAPALAAGNAV
VLKPSEMTPFSTLYMAELSVRAGIPAGLVNVVLGDGPVIGNAITG
HPGIGKVSFTGSTGAGQAIMGNIARNGVKPMTLELGGKSPQIVFA
DADLDLAADCIARSITFNAGQACVAGSRVLVAAEIAEALAERLIA
RMADHRPGITWDAETQYSPIISERQIARIDGIVQAAVAQGAEVLA
GAARLDHPGWFYAPILLAGVAPDSPAVTEEIFGPVLTLEPFADEE
QAVAMADHPTYGLCAGIFTRDLSCALRVMRRIEAGTVWINRYGRS
RDHILPIGGYKSSGIGKDLGRAAYHANRRQKSVLIDL
Exemplary aldehyde dehydrogenase
(SEQ ID NO: 36)
atgacccgatccttcgatcccgacactctggacctgccgcgcgggc
atttcatcgccggcgaacatgtcgccgaccggggccggctggcca
tgcatcgcccctcggacggggcggcgttcggcgagagtccggtcg
cggatgcggatatggtggatcgcgcggtcgcggccgggcgggccg
cgctggccgcctcgggctggggttgcggcgtcccgcgcgaccgca
cccgcgccctgctgaaatgggccgacctgatcgaggccgaggccg
agacgctggcccggttcgaggccgcctgctcgacccgccccgtcg
cgcaattgccggtgggcgacatcgccgtgaccgccgagcagatcc
gctttttcgccgagatggcggacaaggagggcagcgacctggtgc
cgacccgcgacgcctcgctggggatgaccgtggacgagccctatg
gcgtcgtgggcgcgatcacgccctggaattttccgctgtcgatgg
cgggatggaagctggcaccggcgctggcggcaggcaatgcggtgg
tgctgaagccgtccgagatgacgcccttttcgacgctttacatgg
ccgagctttccgtgcgcgcaggcattcccgccgggctggtcaacg
tggtgctgggcgacgggccggtcaccggcaatgcgatcaccggcc
atcccgggatcggcaaggtcagctttaccggctcgaccggggcgg
ggcaggcgatcatgggcaatatcgcccgcaacggcgtcaagccga
tgacgctggaactgggcggcaagtcgccgcagatcgtctttgccg
atgccgacctggatctggccgccgattgcatcgcgcgttccatca
ccttcaacgccgggcaggcctgcgtcgcgggcagccgggtgctgg
tcgcggcggaaatcgccgaggcgctggcggaaaggctgatcgcgc
gcatggccgaccaccgtcccggcaccacatgggatgccgaaacgc
aatattccccgatcatctcggaacgccagatcgcccgcatagacg
gcatcgtgcaggctgccgttgcccaaggggccgaggtgctggccg
gcgcggcgcggctggatcatccgggctggttctatgcgccgacgc
tgctggcgggcgtcgcccccgattcccccgccgtgaccgaagaga
tcttcggcccggtcctgaccttggagccttttgcggacgaagagc
aggccgtcgccatggccgaccatccgacctatggcctttgcgccg
ggatattcacccgcgacctgtcgtgcgcgctgcgcgtcatgcgcc
ggatcgaggccgggacggtctggatcaaccgctacggccgctcgc
gcgaccatatcctgccgacggggggctacaagtcctcgggcatcg
gcaaggatctgggccgcgccgcctatcacgccaaccgccgccaga
aaagcgtgctgatcgacctttga
Exemplary aldehyde dehydrogenase gene-peg.5153

Alcohol dehydrogenase may convert 4-hydroxybutyrate to succinate semialdehyde. In one embodiment, the vector comprises a gene encoding an alcohol dehydrogenase having at least 70%, at least 75%, at least 80%, at least 85%, at least 90%, at least 95%, at least 96%, at least 97%, at least 98%, at least 99% or 100% sequence identity to SEQ ID NO: 37. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 38.

(SEQ ID NO: 37)
MDFTNLGVMRAPRHLVFGAGQRGALARHAGVFGTRALIVTDTRMA
RDKDFLQMRQALEAQGIATQVFDGVAAELPLSCIEAGAKAGRAAG
ARMIIGIGGGSCLDAAKIIGLLLSHGGAPQDYYGEYKVPGPIMPL
ILLPTTSGIGSEVIPVAVLDDPQRAMKIGIASPHLIPEIAICDPE
LTLSCPPGLTAASGADAMTHAIEAFTTLRRPADSGLSLDHVFIGK
NAISDSLALEAIRLIAANLARCVSHGDDLEARSAMMLGSTLAGLA
FGVAGTAAAHAIQYPVGAMTHTAHGLGVATLMPYVMAWNRPSCET
DFARIGAAMGLAASGDISRQAEAAIAAIAALFAQVGIPATIAQLG
VPEDRLDEIARLALSAERLIKNNPRMLDAEGMDRIVRAAHSGDLD
LLTATSPRKAALQ
Exemplary alcohol dehydrogenase
(SEQ ID NO: 38)
atggatttcacgaatttgggcgtcatgcgcgcgccgcggcatctg
gtcttcggcgcgggtcagcgcggcgcgctggcgcgccatgccggt
gtatttggaacccgcgcattgatcgtcaccgatacccgcatggcg
cgggacaaggattttcttcagatgcggcaggccctggaggcgcag
ggcatcgccacgcaggtcttcgacggcgtggccgcggaattgccg
ctgtcctgcatcgaggccggggccaaggccgggcgggcggccggt
gcccggatgattatcggcatcggcggtggcagctgcctggatgcc
gccaagatcatcggcctcttgctgagccatggcggcgcgccgcag
gattattatggcgaatacaaggttcccggtccgatcatgccgctg
atcctgctgcccaccacctcgggaacgggatccgaggtgacgccg
gttgccgtgctggacgacccgcagcgggccatgaagatcggcatt
gccagcccgcatctgatccccgagatcgcgatctgcgacccggag
ctgacgctgagctgtccgccgggcctgaccgcggcctcgggcgcg
gacgccatgacccatgcgatcgaggcgttcacgaccctgcgccgt
cccgccgattccggcctgtcgctggatcatgttttcatcggcaag
aatgcgatcagcgacagcctggcgcttgaggcgatccgcctgatc
gcggccaatctggcccgctgcgtcagccatggcgatgacctggag
gcgcgcagcgcgatgatgcttggctcgaccctggccgggctggcc
ttcggcgtggccggaaccgcggcggcccatgcgatccagtatccg
gtcggcgcgatgacgcataccgcgcatgggctgggggtcgcgacc
ttgatgccctatgtcatggcgtggaaccgccccagttgcgagacc
gatttcgcccggatcggcgccgcgatgggcctggccgcgagtggc
gacacctcccgccaggccgaggccgccatcgccgccattgccgcg
ctttttgcgcaggtgggcattcccgccaccatcgcccagcttggc
gtccccgaggaccggctggacgaaatcgcgcggctggcgctcagc
gccgaacgcctcatcaagaacaacccgcgcatgcttgacgccgaa
ggcatggaccggatcgtccgggccgcgcattcgggcgatcttgac
ctgctgaccgccacatcgccccgaaaggctgcactccaatga
Exemplary alcohol dehydrogenase gene-peg.245

In one embodiment, the vector comprises a gene encoding a succinate-semialdehyde dehydrogenase. Succinate-semialdehyde dehydrogenase (EC 1.2.1.16) is an enzyme that may catalyse the oxidation of succinate-semialdehyde to succinic acid. In one embodiment, the vector comprises a gene encoding a succinate-semialdehyde dehydrogenase having at least 70%, at least 75%, at least 80%, at least 85%, at least 90%, at least 95%, at least 96%, at least 97%, at least 98%, at least 99% or 100% sequence identity to SEQ ID NO: 39. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 40.

(SEQ ID NO: 39)
MSYHDTISQELGFDPQALRGDLWIDGGWRKGRGGDPIAVIDPSIG
NTITRIENASIDDAMDAVAAAEAALPGWAATPPRVKSEILRRCYD
LMIQRKDMLARLISLENGKALPDAQGEVLYAAEFFRWFAEEAVRL
NGEIYTAPSGANRIIVTHRPIGVAVMVTPWNFPAAMATRKIAPAL
AAGCTCVLKPATETPLTAYALAEIYAEAGVPPGVVNVLITSRSGA
TVSAMLHDPRVRKLSFTGSTEVGRRLLHEAADTVISCSMELGGNA
PFIVEDDADLDLAIEGAMVAKMRNGGEACTAANRFLVQKGIAPAF
AERLAARMEAMTLGAGYAGETLCGPLINREALDRIAGLVSEAESH
GAKTLIGGRPLDRPGFYFPPTVLTDVPPQAEITGEEIFGPVAALA
TFETEDEAIARANSTEYGLISYVFTSDLARGLRVSERLDSGMVGL
NRGVVSDPAAPFGGTKQSGLGREGAHHGILEFCEVQYIAANW
Exemplary succinate-semialdehyde dehydrogenase
(SEQ ID NO: 40)
atgagctatcacgacaccatctcgcaagaactcggcttcgacccg
caggcgctgcgcggcgatctgtggatcgacggtggctggcgcaag
gggcgcggcggcgatccgatcgcggtcatcgacccctcgaccggg
aacacgatcacgcggatcgagaatgccagcatcgacgatgccatg
gatgccgtcgccgccgccgaggccgccctgcctggctgggccgcc
accccgccgcgcgtgaaatccgaaatcctgcgccgctgctacgat
ctgatgatccagcgcaaggacatgctggcccggctgataagcctg
gaaaacggcaaggcgctgcccgacgcccagggcgaagtgctctat
gccgccgaattcttccgctggttcgccgaagaggcggtccggctg
aacggcgagatctacaccgcgccctcgggtgcgaaccgcatcatc
gtcacccaccgcccgatcggggtcgcggtgatggttacgccctgg
aacttccccgccgccatggccacccgcaagatcgcgcccgcgctg
gcggcgggctgcacctgcgtgctgaagcccgcgacggaaaccccg
ctgaccgcctatgcgctggccgagatctatgccgaggccggtgtg
ccgccgggcgtggtcaacgtgctgaccaccagccgttcgggcgcg
acggtcagcgcgatgctgcacgatccgcgcgtccgcaagctcagc
ttcaccggctcgaccgaggtggggcgcaggctcttgcacgaggcg
gccgatacggtcatctcctgctcgatggaactgggcggcaacgcg
cccttcatcgtcttcgacgacgcagatctggacctggcgatcgag
ggcgcgatggtcgccaagatgcgcaacggaggcgaggcctgcacc
gccgccaaccgtttcctggtgcagaagggcatcgcaccggccttc
gccgaacggcttgccgcgcggatggaggccatgacgctgggggcg
ggctatgccggggaaaccctttgcgggccgctgataaatcgtgag
gcgcttgatcgcatcgcgggcctggtgagcgaggccgaaagccac
ggtgcgaagaccctgaccggcggtcgccccctggaccggccgggc
ttctatttcccgccgaccgtcctgaccgatgtgccgccccaggcc
gagatcaccggcgaggaaatcttcggccccgtcgcggccctggcc
accttcgagaccgaggacgaggccatcgcccgcgccaattcgacc
gaatacgggctgatctcctatgttttcacctcggacctggcgcgg
gggctgcgcgtgtcggaacggctggacagcggcatggtggggctc
aatcgcggcgtggtctcggacccggcggcgccgttcggcggcacc
aagcaaagcgggcttggccgggaaggcgcccatcacggcatcctc
gaattctgcgaagtccagtatatcgccgccaactggtga
Exemplary succinate-semialdehyde dehydrogenase
gene peg.246

In one embodiment, the vector comprises a gene encoding a succinate-semialdehyde dehydrogenase having at least 70%, at least 75%, at least 80%, at least 85%, at least 90%, at least 95%, at least 96%, at least 97%, at least 98%, at least 99% or 100% sequence identity to SEQ ID NO: 41. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 42.

(SEQ ID NO: 41)
MTKHATDLKMLLKDPSLLQTRAYVAGEWVDADDGKIFPVVNPARG
DVIAEVADLSRAEVARAIAAAAEAMKGWAARTAKGRAQIMRKWFD
LMMENQDDLGRILTAEMGKPLPEAKGEIAYGASFIEWEGEEAKRI
YGETIPGHLPDKRLTVIRQPIGVVGSITPWNFPNAMITRKCGPAI
AAGCGFVGRPAAETPLSALALAVLAERAGIPKGLFSIVISSRSSD
IGKEFCENPLIRKLIFTGSTEVGRILLRQAADQVLKCSMELGGNA
PFIVEDDADLDAAVEGAMASKERNNGQTCVCANRIYVQAGVYDAF
AQKLAAAVDKLRVGDGLEEGVITGPLINQDAVEKVQEHIQDAVAG
GATVVTGGKPREGLFFDPTVVIGITDKMKVATEETFGPLAPLFRF
ETEEEAVERANATIFGLASYFYARDIGRITRVQEALEYGIVGVNT
GIISTEVAPFGGVKQSGLGREGSRHGIEDYLEMKYICLSI
Exemplary succinate-semialdehyde dehydrogenase
(SEQ ID NO: 42)
atgaccaagcatgcgaccgacctgaagatgctgctcaaagacccc
tcgctcttgcagacccgcgcctatgtcgccggcgaatgggtcgat
gccgacgacggcaagaccttcccggtggtgaacccggcacgcggc
gacgtgatcgccgaggtggcggacctgagccgcgccgaggtcgcc
cgcgccatcgcggccgccgccgaggcgatgaagggctgggccgcg
cgcaccgccaagggacgcgcccagatcatgcgcaaatggttcgac
ctgatgatggagaaccaggacgacctgggccgcatcctgaccgcc
gagatgggcaagccgctgcccgaggccaagggcgagatcgcctat
ggcgccagcttcatcgaatggttcggcgaagaggccaagcgcatc
tatggcgagaccatccccggccacctgcccgacaagcgcctgacg
gtgatccgccagccgatcggggtggtgggctcgatcacgccctgg
aactttcccaatgcgatgatcacccgcaaatgcggcccggccatc
gccgccggctgcggcttcgtcggccgtcccgccgccgagacgccg
ctttcggccctggcgctggccgttctggccgagcgcgccggcatc
cccaaggggctgttcagcatcgtgacctcgtcgcggtcatcggac
atcggcaaggaattctgcgagaacccgctgatccgcaagctgacc
ttcaccggctcgaccgaggtcggtcgcatcctgctgcggcaggcc
gccgaccaggtgctgaaatgctcgatggagctgggcggcaacgcg
cccttcatcgtcttcgacgacgccgacctggacgcggcggtcgag
ggggccatggcctcgaaattccgcaacaacggccagacctgtgtc
tgcgccaaccgcatctatgtgcaagccggcgtctatgacgcattc
gcgcaaaagctggccgcggcggtggacaagctgcgcgtcggcgac
gggctggaagagggcgtgaccaccggcccgctgatcaaccaggat
gcggtggaaaaggtccaggagcatatccaggacgccgtcgcgggc
ggcgccaccgtggtcaccggcggcaagccgcgcgaggggctgttc
ttcgacccgaccgtggtcaccggcatcaccgacaagatgaaggtg
gcgaccgaagagaccttcggcccgctcgcgccgctgttccggttc
gagaccgaggaagaggccgtcgagcgcgccaatgcgaccatcttc
ggccttgcctcgtatttctatgcccgcgacatcggccgcatcacc
cgggtgcaggaggcgctggaatacggcatcgtgggcgtgaatacc
ggcatcatctcgaccgaggtggcgcccttcggcggcgtcaagcaa
tccggcctgggccgcgagggctcgcgccacggcatcgaggattac
ctggagatgaaatacatctgcctatcgatctga
Exemplary succinate-semialdehyde dehydrogenase
gene-peg.2316

In one embodiment, the vector comprises a gene encoding a succinate-semialdehyde dehydrogenase having at least 70%, at least 75%, at least 80%, at least 85%, at least 90%, at least 95%, at least 96%, at least 97%, at least 98%, at least 99% or 100% sequence identity to SEQ ID NO: 43. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 44.

(SEQ ID NO: 43)
MTALKDKDLLRQQALIGGNWVDAASGAVVQVTDPATGQVMGTIPD
LSAAETRAAIDAADAAFASWKKRSHAERAALLERWFDLMNQHAED
LALILTLEQGKPLSEARGEIAYGASFVKWFAEEARRIDGTVIPAP
INDRRILILKEPVGVSAIITPWNFPNAMITRKVGPALAAGCTVVI
KPSEFTPYSALALGVLAERAGIPAGVVNIVTGMPAEIGAELTANP
TVRKVSFTGSTRVGSLLMAQSAPTVKRLSLELGGNAPFIVEDDAD
LDAAVEGAIASKFRNGGQTCVCSNRILVQAGVYDAFAEKLGAKVA
AMKVGPGTQAGNDIGPMINRAALDKIARHVADAVAKGATVAARAE
IPEGQYAAPVVLIGATTEMELASEETFGPVAPLFRFETEDEAVAI
ANGTPFGLAAYFYTENIRRAWRVAEALEFGMVGLNTGAVSTTVSP
FGGVKSSGLGREGARAGIEEYLEVKAFHMGGL
Exemplary succinate-semialdehyde dehydrogenase
(SEQ ID NO: 44)
atgacggcattgaaggacaaggatctgcttcgccagcaggcgctga
tcggcggcaattgggtcgatgcggccagcggcgccgtggtccagg
tcaccgaccccgcgaccgggcaggtcatgggcaccattcccgacc
tgtcggccgccgagacccgcgcggccatcgatgccgccgatgccg
ccttcgcctcgtggaaaaagcgcagccatgccgagcgcgcggcgc
tgctggaacgctggttcgacctgatgaaccagcatgccgaggatc
tggcgctgatcctgacgctggaacagggcaagccgctttccgagg
cacggggggaaatcgcctatggcgcctctttcgtgaaatggttcg
ccgaggaggcgcggcgcatcgacggcaccgtgatcccggcgccga
cgaacgaccgccgtatcctgacgctgaaggagccggtcggcgtct
cggccatcatcacgccgtggaacttcccgaacgcgatgatcaccc
gcaaggtcggcccggccctggcggcgggctgcaccgtggtcatca
agccttccgagttcacgccttattccgcgctcgccctgggcgttc
tggccgagcgcgcggggattccggcgggcgtcgtcaacatcgtca
ccgggatgccggccgagatcggcgcggaactgaccgccaacccga
cggtgcgcaaggtcagctttaccggctcgacccgcgtcggctcgc
ttctgatggcgcaatccgcgccgacggtaaagcggctgtcgctgg
aactgggcggcaacgcgcccttcatcgtcttcgacgatgccgatc
tggacgcggcggtcgagggggcgatcgcgtccaagttccgcaatg
gcggccagacctgcgtctgctcgaaccggatcctggtgcaggcgg
gcgtctacgacgcctttgccgaaaagctgggcgccaaggtcgcgg
cgatgaaggtcggtcccggcacgcaggccggcaacgacatcggcc
cgatgatcaaccgcgctgcgctggacaagatcgcgcgccatgtcg
cggatgccgtggcgaaaggcgcgaccgttgccgcccgggccgaga
tccccgaggggcaatatgccgcccccgtggtcctgacgggtgcca
cgaccgagatggagctggcatccgaggaaaccttcggccccgtcg
cgccgcttttccgctttgagaccgaggacgaggccgtcgccatcg
cgaacggcacccccttcggccttgccgcctatttctataccgaga
atatccgccgtgcctggcgcgtggccgaggcgctggaattcggca
tggtggggctgaacaccggcgcggtctcgaccaccgtttcgcctt
ttggcggcgtcaagtcctcgggcctgggccgcgagggcgcgcggg
cggggatcgaggaatatctggaggtgaaggccttccacatgggcg
ggctctga
Exemplary succinate-semialdehyde dehydrogenase
gene-peg.2992

### PHA Synthesis Pathway

In one embodiment, the vector comprises one or more genes encoding all or part of a pathway for the synthesis of PHA. Suitably, the vector comprises a gene encoding a 3-ketoacyl-CoA thiolase, an enoyl-CoA hydratase, and/or a PHA synthase.

3-ketoacyl-CoA thiolase may catalyse the condensation of 2 acetyl-CoA molecules to form acetoacetyl-CoA. In one embodiment, the vector comprises a gene encoding a 3-ketoacyl-CoA thiolase having at least 70%, at least 75%, at least 80%, at least 85%, at least 90%, at least 95%, at least 96%, at least 97%, at least 98%, at least 99% or 100% sequence identity to SEQ ID NO: 45. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 46.

(SEQ ID NO: 45)
MSTDPIVIVGSARTPMGGFQGDFAGVEAAALGATAIKAALGGLDP
QAVDEIIMGCVLPAGQGQAPARQAALGAGLPLGAGATTVNKMCGS
GMKAAMLGHDLILAGSADVVVAGGMESMSNAPYLLPKARSGYRMG
HGQVMDHMFLDGLEDAYDKGRLMGTFAEDCAEAYQFTREAQDEFA
ISSLTRAQKAIAAGHFTGEIAPVTVRGRGGETVVDIDEQPGKARP
DKIPTLRPAFRKDGTVTAANSSSISDGAAALVLMRASEAERRGLV
PRARILGHAIFADKPGLFPTAPIGSVRRLLERIGTAIGDYDLFEV
NEAFAVVAMAAMRDLGLSHDAVNVHGGACALGHPIGASGARVLVI
LLAALETHGGRRGIASLCIGGGEATAVAIERMQ
Exemplary 3-ketoacyl-CoA thiolase
(SEQ ID NO: 46)
atgagcaccgatccgattgtcatcgtgggctcggcccgcacgccg
atgggcggcttccagggcgatttcgccggggtcgaggccgccgcg
ctgggcgcgaccgcgatcaaggcggcgctgggcgggctggacccg
caggcggtggacgagatcatcatgggctgcgtgctgcccgccggc
cagggccaggccccggcgcggcaggcggcgctgggcgcgggcctg
cccttgggcgccggcgccacgaccgtcaacaagatgtgcggatcg
ggcatgaaggcggcgatgctgggccatgacctgatcctggccggt
tccgccgatgtggtggtggcgggcggcatggagagcatgtcgaac
gcgccctatctgctgccgaaggcgcggtcgggctatcgcatgggg
catgggcaggtgatggaccacatgttcctggacgggctggaggac
gcctatgacaagggccggctgatgggcacattcgccgaggattgc
gccgaagcctatcagttcacgcgcgaggcgcaggacgagttcgcg
atttcctcgctgacccgggcgcagaaggccatcgccgcggggcat
ttcacgggcgagatcgcgccggtgacggtcagggggcgcggcggc
gagacggtcgtggataccgacgagcagccgggcaaggcgcggccg
gacaagatcccgaccttgaggccggcctttcgcaaggatggcacg
gtgacggcggcgaacagctcctcgatctcggacggggcggcggcg
ctggtgctgatgcgggcgtcggaggccgagcggcgcgggctggtg
ccgcgggcgcggatccttgggcacgcgacctttgccgacaagccg
ggcctgtttccgaccgcgcccatcggctcggtccggcggttgctg
gagcggacggggacggcgatcggcgattacgacctgttcgaggtc
aacgaggctttcgcggtggtcgccatggcggcgatgcgcgacctt
ggcctgtcgcatgatgcggtgaacgtgcatggcggcgcctgcgcg
ctgggccatcccatcggcgcctcgggcgcgcgggtgctggtgacg
ctgctggcggcgctggagacgcatgggggccggcgcggcatcgcg
tcgctgtgcatcggcgggggcgaggcgacggcggtggccatcgag
aggatgcaatga
Exemplary 3-ketoacyl-CoA thiolase gene-peg.2992

In one embodiment, the vector comprises a gene encoding an enoyl-CoA hydratase. Enoyl-CoA hydratase (EC 4.2.1.17) may also function as Delta (3)-cis-delta (2)-trans-enoyl-CoA isomerase (EC 5.3.3.8), 3-hydroxyacyl-CoA dehydrogenase (EC 1.1.1.35), and/or 3-hydroxybutyryl-CoA epimerase (EC 5.1.2.3), and may function to synthesise hydroxybutyryl-CoA. In one embodiment, the vector comprises a gene encoding an enoyl-CoA hydratase having at least 70%, at least 75%, at least 80%, at least 85%, at least 90%, at least 95%, at least 96%, at least 97%, at least 98%, at least 99% or 100% sequence identity to SEQ ID NO: 47. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 48.

(SEQ ID NO: 47)
MPVHYDLAGDSAVLTFDNPPLNVLGQAMRADLARAIAQAAADRPA
RLILRGAGRNFVAGADAREFDGPPLDPQLNEVLDALAALPFPTIA
AIHGAALGGGLEIALACRFRIAHPSAILGLPEVTLGIVPGAGGTQ
RLPRLVGMAAALDLLGQGRSVTAAEAESLGLIDLIADDPMAAARG
VDTQTLLRALCADDRPPPAPDEAAVAAAHARADRRAPGQVAPHRA
IELVATSAQEPIKAALTRERATFLDLRGSDQARALRHVFFAERAA
MAQGKAWPAPAPEIARAVVVGGGNMGAAIAYALLSAGLVVRVVET
DAAALDRARDNIAGLVAQGRKRGALTDAGAAELQARLSLAVGYDD
LPAADLAIEAAYEDMAVKQAIFAALQDALPDSTILATNISYLDID
LLAQGIRQPGRFLGLHFFAPAHVMKLLEIVRGEATSDQTLGAAFR
LARKLGKVPVLAGVCDGFIGNRILARYRHAADILLLEGALPAQVD
AAMRGFGMAMGPYEAQDMSGLDIAYANRRRQNLRDRADHRYVPIA
DHLVERCRRLGRKSGAGWYDYDAEGRAQPSDEVIQAILSASRDAG
ITRVALPAEGIAERLVLAMIAEATRILAEGIAAAPRDIDLVLVHG
YGFPRWRGGLMHHADRLTPARILSRIEALAKDDPLSWSVPPLLRQ
LADEGRDFTSLNPSA
Exemplary enoyl-CoA hydratase
(SEQ ID NO: 48)
atgccggtgcattacgacctcgccggcgacagcgccgtcctgacc
ttcgacaatccgccgctgaacgtgctgggccaggcgatgcgcgcc
gatctggctcgcgccatcgcgcaggcggcggccgaccgccccgcc
cggctgatcctgcgcggggcggggcgcaatttcgtcgccggggcg
gatgcgcgcgaattcgacggcccgccgctggacccgcagctgaac
gaggtgctggacgcgctggccgccctgcccttcccgaccatcgcc
gcgatccatggcgcggcccttggcggcgggctggagatcgcgctg
gcctgccgcttccgcatcgcccacccctcggccacgctcggcctg
cccgaagtcaccctgggcatcgtgcccggcgcgggcggcacgcag
cggttgccgcggctggtggggatggcggccgcgctggacctgttg
ggacagggccgcagcgtgaccgccgccgaagcggaaagcctgggc
ctgatcgacctgatcgccgatgacccgatggccgccgcgcggggc
gtggacacgcagaccctgctccgcgccctctgcgccgacgaccgc
ccgccccccgcgccggacgaggcggcggtcgcggcggctcatgcg
cgggccgaccggcgcgcaccgggccaggtcgcgccgcaccgggcg
atcgagctggtggccacctcggcgcaagaaccgatcaaggcggcg
ctgacacgcgagcgcgcgaccttcctcgacttgcgcggatcggac
caggcgcgggcgctgcgccatgtgttcttcgccgaacgcgccgcc
atggcgcagggaaaggcctggcccgcccccgcgccagagatcgcc
cgcgccgtggtggtcggcggcggcaatatgggcgcggccatcgcc
tatgcgctgctgtcggccgggctggtggtcagggtggtcgagacc
gacgccgccgcgctggaccgcgcccgcgacaatatcgccgggctg
gtggcgcagggccgcaagcgcggcgcgctgaccgatgccggcgcc
gccgagttgcaggcgcggctgtcgctggcggtgggctatgacgac
ctgcccgccgccgacctggccatcgaggccgcctatgaggacatg
gcggtcaagcaggcgatcttcgccgcgctgcaggacgcgctgccc
gacagtacgatcctggccacgaatacctcgtatctggatatcgac
ctgctggcgcagggcatccggcagccgggccgcttcctggggctg
catttcttcgcgcccgcccatgtgatgaagctgctggagatcgtg
cgcggcgaggcgacgtcggaccagaccctgggcgcggcgttccgg
ctggcgcggaagcttggcaaggtgccggtgctggcgggcgtctgc
gacgggttcatcggcaaccgcatccttgcccgctatcgccacgcg
gccgacatcctgctgctggaaggcgcgctgccggcccaggtcgat
gccgccatgcgcggctttggcatggcgatggggccatatgaggcg
caggacatgtcggggctggacatcgcatacgccaaccgccgccgc
cagaacctgcgcgaccgcgctgaccaccgctacgtcccgatcgcc
gatcatctggtcgagcgttgcaggcgcctgggccgcaagtccggc
gcggggtggtatgactatgatgccgaaggccgcgcgcagccttcg
gacgaggtgacgcaggcgatcctgtcggccagccgcgatgccggc
atcacccgcgtggcgctgcccgccgagggcatcgccgaacggctg
gtcctggcgatgatcgccgaggcgacccggatcctggccgagggc
atcgccgccgcgccgcgcgacatcgacctggtgctggtccatggc
tacggctttccgcgctggcgcggcgggctgatgcaccatgccgac
cggctgacgcccgcgcgcatcctgtcccggatcgaggccctggcc
aaggacgacccgctgtcctggtccgtcccgccgcttctgcgccag
cttgcggatgaagggcgcgatttcaccagcctgaacccatccgcc
tga
Exemplary enoyl-CoA hydratase gene-peg.203

In one embodiment, the vector comprises a gene encoding a PHA synthase. In one embodiment, the vector comprises a gene encoding a PHA synthase having at least 70%, at least 75%, at least 80%, at least 85%, at least 90%, at least 95%, at least 96%, at least 97%, at least 98%, at least 99% or 100% sequence identity to SEQ ID NO: 49. In one embodiment, the vector comprises a gene comprising or consisting of a nucleotide sequence having at least 70%, at least 75%, at least 80%, at least 85%, at least 90%, at least 95%, at least 96%, at least 97%, at least 98%, at least 99% or 100% sequence identity to SEQ ID NO: 50.

(SEQ ID NO: 49)
MAGKDKKPEVEAGAGDAAKPRKARGTGAKARSAAKVAKQDPAPAD
SEAPARGRRAKAAPAAKAAQEPVTLAKPKAAAGSGAGKPAAEPVA
KSGARKPVSRARAASRSDPRTGTRRKPSAKAAAKAQAEAASLSAV
DEALRPLGAVGPGAAPPMAAPAAASPTPAGTERPAAPAAEPSSAA
AFAEAAFGIGSRLPEQLAQNIERIESLIQRLISALAQRRPHSPGV
ELPGPELFATATGAWIKLLTEQPERVLSQQVSYWGETLRHFAEAQ
AALARGTLKPPPSEGLRDRRFSNPLWEAHPFFNFIKRQYQINAQA
LEEAASALDLPEMTDRRRIEWFTRQMIDMMAPINFLATNPDALEK
ALETEGESLVKGLENLVRDVEQNNGELIVSLADRDAFRVGENIGI
TEGIVVARIKLYELIQYKPTTAQVHEIPLVIFPPWINKFYILDLK
PQNSLIKWIVDQGYILFVVAWKNPDPSYGDTGMDDYVTAYLEVMD
RVLDLIDQKKLNVVGYCIAGTTLALILSILKQRGDDRVNSATFFT
ALTDFADQGEFTAYLQEDFVSGIEEEAARTGVLGAQLMTRIFSFL
RANDLVWGPAIRSYMLGEMPPAFDLLFWNGDGINLPGRMAVEYLR
GLCQQNRFVKEGFDLLGHRLHVGDVTVPLCAIACETDHIAPWRDS
WRGVAQMGSKDKIFILSESGHIAGIVNPPSKKKYGHYTSDAGFDQ
GEQHWLDKARHHEGSWWGRWGEWLARRAGNMVEARDPGEGFGPAP
GLYVHERA
Exemplary PHA synthase
(SEQ ID NO: 50)
atggcgggcaaggacaaaaaaccggaagtcgaagccggggccggcg
atgcggccaaaccgcgcaaggctcgcgggactggggcaaaggccc
gatccgcggccaaggtcgcgaaacaggacccggcccccgccgatt
cggaagcgccggcgcgcggccgccgggcgaaggctgcccccgctg
ccaaggccgcgcaggagcccgtgacgctggccaagcccaaggcgg
ccgccggatccggggccggcaagcctgccgccgagccggttgcca
agtccggcgcccgcaagcccgtgtcgcgtgcgcgggcggccagcc
gcagcgatccccgcaccggcacgcgccgcaagccctctgccaagg
ccgccgcgaaggcgcaggccgaagccgcatcgctgagcgcggtcg
acgaggcgctgcgcccgcttggcgccgtcgggccgggtgccgcgc
cgcccatggccgcgcccgctgccgcgagcccgacccccgccggga
ccgaaagacccgccgcccccgctgccgagccgtccagcgccgccg
ccttcgccgaagccgccttcggcatcggcagccgcctgcccgagc
agctggcccagaacatcgaacgtatcgaatcgctgacccagcgcc
tgatcagcgcgctggcgcagcgccgcccacacagccccggcgtcg
aactgcccggtcccgaactgttcgccaccgcgaccggcgcctgga
tcaagctgctgaccgagcagcccgagcgcgtgctcagccagcagg
tcagctattggggcgagacgctgcgccatttcgccgaggcccagg
ccgcccttgcccgcggcacgctgaagccgccgcccagcgagggac
tccgggaccggcgcttctcgaacccgctctgggaggcgcatccgt
tcttcaacttcatcaagcggcaataccagatcaacgcccaggcgc
tggaagaggcggccagcgcgctcgacctgcccgagatgaccgacc
ggcgccggatcgaatggttcacccgccagatgatcgacatgatgg
cgccgacgaatttcctggcgaccaatcccgacgcgctggaaaagg
cgctggagaccgagggcgaaagcctggtcaagggccttgagaacc
tggtgcgtgacgtcgagcagaacaatggcgagctgatcgtctcgc
tggccgaccgcgacgccttccgcgtgggcgagaacatcggcacca
ccgagggcacggtggtggcgcgcaccaagctttacgagctgatcc
agtacaagcccaccaccgcccaggtgcacgagatcccgctggtaa
tctttccgccctggatcaacaagttctacatcctcgacctcaagc
cgcagaacagcctgatcaaatggatcgtcgatcagggctatacgc
tgttcgtggtggcctggaagaaccccgatcccagctatggcgata
ccggcatggacgattacgtcaccgcctatctggaggtgatggacc
gggtgctggacctgaccgaccagaaaaagctgaacgtcgtcggct
attgcatcgccgggacgacgctggcgctgacgctgtcgatcctca
agcagcgcggcgacgaccgggtgaattcggccaccttcttcactg
cgctgaccgatttcgcggatcagggcgagttcaccgcctatctgc
aagaggatttcgtctcgggcatcgaggaggaggcggcgcggaccg
gcgtgcttggcgcgcagctgatgacgcgcaccttcagcttcctgc
gcgccaacgacctggtctgggggccggcgattcgcagctatatgc
tgggcgagatgccgcccgcgttcgacctgctgttctggaacggcg
acggcaccaacctgcccgggcgcatggcggtggaatacctgcggg
gcctgtgccagcagaaccgcttcgtcaaggaggggttcgacctgc
tgggccaccgactgcatgtcggtgacgtcaccgtgccgctttgcg
ccatcgcctgcgagacggatcatatcgcgccctggcgcgacagct
ggcgcggcgtggcgcagatgggctcgaaggacaagaccttcatcc
tgtccgaatcgggccatatcgccggcatcgtcaatccgcccagca
agaagaaatacggccattacacctcggatgccggtttcgatcagg
gcgagcagcactggctggacaaggccaggcatcacgagggcagct
ggtggggccgctggggcgagtggctggcccgccgggcggggaaca
tggtcgaggcccgcgatccgggcgagggcttcggccccgcgcccg
ggctctacgttcacgagcgggcataa
Exemplary PHA synthase gene-peg.988

### Cells and Kits

In one aspect, the present invention provides a cell comprising the vector according to the present invention. The cell may be an isolated cell.

The cell may be a microbe, such as a bacteria, archaea, fungi or protist. In some embodiments, the cell is a bacterium. In some embodiments, the bacterium is from the family Rhodobacteraceae. In some embodiments, the bacterium is from the genus Paracoccus. In some embodiments, the bacterium is a Paracoccus denitrificans, Paracoccus pantotrophus, or Paracoccus versutus. In some embodiments, the bacterium is a Paracoccus denitrificans.

In one aspect, the present invention provides a kit for producing polyhydroxyalkanoate (PHA) from polyester waste.

The kit may comprise one or more microbes according to the present invention. The kit may comprise one or more vectors according to the present invention. The kit may comprise instructions for performing the method of the present invention.

### EXAMPLES

The invention will now be further described by way of Examples, which are meant to serve to assist one of ordinary skill in the art in carrying out the invention and are not intended in any way to limit the scope of the invention.

### Example 1: Generation of a Genome Scale Model (GSM)

The present inventors identified genetic traits that allow the metabolic conversion of various biodegradable polyester waste and their monomers, respectively, to form new biologically synthesized polyester (PHA) by the same microorganism (e.g. Paracoccus denitrificans).

Genetic traits were identified by newly generating a Genome Scale Model (GSM) based on the publicly available genome of P. denitrificans PD1222 (see GenBank assembly accession: GCA_000203895.1). P. denitrificans PD1222 is a derivative of P. denitrificans DSM 413. The GSM was used for metabolic reconstruction by identifying in the genome the genes described previously in different microorganisms for the catabolism of each of the polyester components, as well as the genes identified to produce the PHA polyester PHB. With the GSM, we can go beyond sole identification of genes present in the genome but also see the possible metabolic network formations in the organism, therefore identifying plausible connections between the activity of the different genes for a metabolic outcome. The targeted components were most of the monomers employed to produce the main plastic polyesters industrially.

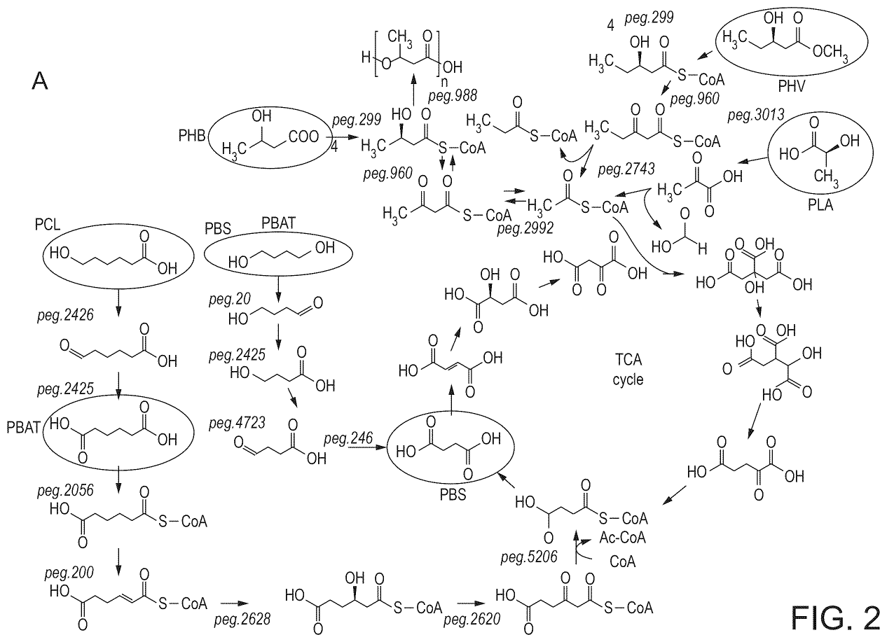

Table 1 shows the genetic traits and network connections identified in the genome of which are relevant for the utilization of a vast array of polyester monomers via aerobic or anoxic metabolic routes in Paracoccus denitrificans. Table 1 also shows the genetic traits and network connections identified which are relevant for the production of PHA (in the form of PHB) by Paracoccus denitrificans. FIG. 2 shows a visualization of the metabolic capabilities for conversion of polyester monomers by Paracoccus denitrificans based on the newly constructed GSM (Genome Scale Model)

The present inventors found for the first time the pathway for 1,4-Butanediol is present in this microorganism. Specifically, 1,4-Butanediol is one of the constitutive monomers of a variety of polyesters and has also toxic effects for humans, and is therefore highly relevant for achieving biological degradation and recycling.

In the following examples, based on the newly constructed GSM, a derivative of P. denitrificans DSM 413 was used.

TABLE 1
Summary table of identified genes that when present in a genome allow for the metabolic utilization
of different polyester monomer components and subsequent production of a bio-polyester (PHB). These genes
are part of the metabolic routes identified in P. denitrificans PD1222 and are also present in other
P. denitrificans including, for example, P. denitrificans ATCC 19367 (see Si, Y.Y., et al., 2019.
Canadian journal of microbiology, 65(7), pp.486-495), P. denitrificans DYTN-1 (see Zhao, Y., et al.,
2020. Letters in applied microbiology, 70(4), pp.263-273), and P. denitrificans R-1 (see Hu, C.,
et al., 2022. Microbiology Resource Announcements, 11(4), pp.e01236-21).

| Polyester | | | | Gene present in |
|---|---|---|---|---|
| monomer | Gene ID | Enzyme annotation | Function | P. denitrificans strain |
| Succinic | peg.3652 | Succinate | Transforms succinate into | PD1222; ATCC |
| acid | | dehydrogenase/fumarate | fumarate | 19367; DYTN-1; R-1 |
| | | reductase, flavoprotein | | |
| | | subunit | | |
| Lactic | peg.3013 | D-lactate dehydrogenase (EC | Transforms lactate into | PD1222; ATCC |
| acid | | 1.1.1.28) | pyruvate. | 19367; DYTN-1; R-1 |

Ethylene glycol

| | | | | |
|---|---|---|---|---|
| Step 1 | peg.4723 | Alcohol dehydrogenase (EC | Transforms ethylene glycol | PD1222; ATCC |
| | | 1.1.1.1) | into glycolaldehyde | 19367; DYTN-1; R-1 |
| Step 2 | peg.4722 | Aldehyde dehydrogenase (EC | Transforms glycolaldehyde | PD1222; ATCC |
| | | 1.2.1.3) | into glycolate | 19367; DYTN-1; R-1 |
| Step 3 | peg.1987 | Glyoxylate reductase (EC | Transforms glycolate into | PD1222; ATCC |
| | | 1.1.1.79) @ Glyoxylate | glyoxylate | 19367; DYTN-1; R-1 |
| | | reductase (EC 1.1.1.26) @ | | |
| | | Hydroxypyruvate reductase | | |
| | | (EC 1.1.1.81) | | |

Adipic acid

| | | | | |
|---|---|---|---|---|
| Step 1 | peg.2056 | Long-chain-fatty-acid--CoA | Ligates Acetyl-CoA to adipic | PD1222; ATCC |
| | | ligase (EC 6.2.1.3) | acid | 19367; DYTN-1; R-1 |
| Step 2 | peg.200 | Acyl-CoA dehydrogenase | Transforms adipyl-CoA into | PD1222; ATCC |
| | | | 5-Carboxy-2-pentenoyl- | 19367; DYTN-1 |
| | | | CoA | |
| Step 3 | peg.2628 | Enoyl-CoA hydratase (EC | Hydrates enoyl CoA to 3- | PD1222; ATCC |
| | | 4.2.1.17) | hydroxybutyryl-CoA | 19367; DYTN-1; R-1 |
| Step 4 | peg.2620 | 3-hydroxybutyryl-CoA | Transforms 3- | PD1222; ATCC |
| | | dehydrogenase (EC | hydroxybutyryl-CoA into 3- | 19367; DYTN-1; R-1 |
| | | 1.1.1.157) | oxoadipyl-CoA | |
| Step 5 | peg.5206 | 3-oxoadipyl-CoA thiolase (EC | Transforms 3-oxoadipyl- | PD1222; ATCC |
| | | 2.3.1.174) | CoA into succinyl-CoA | 19367; DYTN-1; R-1 |

6-Hydroxycaproic acid

| | | | | |
|---|---|---|---|---|
| Step 1 | peg.2426 | Alcohol dehydrogenase | Transforms 6- | PD1222; ATCC |
| | | (EC.1.1.1.1) | hydroxycaproic acid into 6- | 19367; DYTN-1; R-1 |
| | | | oxocaproic acid. | |
| Step 2 | peg.2425 | Aldehyde dehydrogenase (EC | Transforms 6-oxocaproic | PD1222; ATCC |
| | | 1.2.1.3) | acid into adipic acid. | 19367; DYTN-1; R-1 |

3-Hydroxybutyric acid

| | | | | |
|---|---|---|---|---|
| Step 1 | Peg.2994 | Acyl-CoA synthetases (AMP- | Synthesizes 3- | PD1222; ATCC |
| | | forming)/AMP-acid ligases | hydroxybutyril-CoA | 19367; DYTN-1; R-1 |
| Step 2 | peg.960 | 3-hydroxybutyrate | Transforms 3- | PD1222; ATCC |
| | | dehydrogenase (EC 1.1.1.30) | hydroxybutyrate into | 19367; DYTN-1; R-1; |
| | | | acetoacetate. | NBRC13301 |

3-Hydroxyvaleric acid

| | | | | |
|---|---|---|---|---|
| Step 1 | Peg.2994 | Acyl-CoA synthetases (AMP- | Synthesizes 3- | PD1222; ATCC |
| | | forming)/AMP-acid ligases | hydroxyvaleryl-CoA | 19367; DYTN-1; R-1 |
| Step 2 | peg.960 | 3-hydroxybutyrate | Transforms 3- | PD1222; ATCC |
| | | dehydrogenase (EC 1.1.1.30) | hydroxyvalerate into 3- | 19367; DYTN-1; R-1; |
| | | | oxopentanoic acid. | NBRC13301 |
| Step 3 | peg.2743 | 3-ketoacyl-CoA thiolase (EC | Breaks down 3- | PD1222; ATCC |
| | | 2.3.1.16) | oxopentanoyl-CoA into | 19367; DYTN-1; R-1 |
| | | | acetyl-CoA and propionyl- | |
| | | | CoA. | |

1,4-Butanediol

| | | | | |
|---|---|---|---|---|
| Step 1 | peg.20 | Methanol dehydrogenase | From, 1.4-butanediol to 4- | PD1222; ATCC |
| | | (EC 1.1.2.7) | hydroxybutyraldehyde. | 19367; DYTN-1; R-1 |
| | peg.3083 | | | PD1222; ATCC |
| | | | | 19367; DYTN-1; R-1 |
| Step 2 | peg.2425 | Aldehyde dehydrogenase | From 4- | PD1222; ATCC |
| | | (EC1.2.1.3) | hydroxybutyraldehyde to | 19367; DYTN-1; R-1 |
| | peg.5153 | | 4-hydroxybutyrate | PD1222; ATCC |
| | | | | 19367; DYTN-1; R-1 |
| Step 3 | peg.245 | Alcohol dehydrogenase | From 4-hydroxybutyrate to | PD1222; ATCC |
| | peg.4723 | (EC.1.1.1.1) | succinate semialdehyde | 19367; DYTN-1 |
| | | | | PD1222; ATCC |
| | | | | 19367; DYTN-1; R-1 |
| Step 4 | peg.246 | Succinate-semialdehyde | Catalyzes de oxidation of | PD1222; ATCC |
| | | dehydrogenase (EC1.2.1.16) | succinate-semialdehyde to | 19367; DYTN-1 |
| | peg.2316 | | succinic acid. | PD1222; ATCC |
| | | | | 19367; DYTN-1; R-1 |
| | peg.5151 | | | PD1222; ATCC |
| | | | | 19367; DYTN-1 |

Production of PHB

| | | | | |
|---|---|---|---|---|
| Step 1 | peg.2992 | 3-ketoacyl-CoA thiolase (EC | Catalyzes the condensation | PD1222; ATCC |
| | | 2.3.1.16) | of 2 acetyl-CoA molecules | 19367; DYTN-1; R-1 |
| | | | to form acetoacetyl-CoA | |
| Step 2 | peg.203 | Enoyl-CoA hydratase (EC | Synthesizes 3- | PD1222; ATCC |
| | | 4.2.1.17) / Delta(3)-cis- | hydroxybutyryl-CoA | 19367; DYTN-1 |
| | | delta(2)-trans-enoyl-CoA | | |
| | | isomerase (EC 5.3.3.8)/3- | | |
| | | hydroxyacyl-CoA | | |
| | | dehydrogenase (EC 1.1.1.35)/ | | |
| | | 3-hydroxybutyryl-CoA | | |
| | | epimerase (EC 5.1.2.3) | | |
| Step 3 | peg.988 | Polyhydroxyalkanoic acid | Synthesizes PHB | PD1222; ATCC |
| | | synthase | | 19367; DYTN-1; R-1 |

### Example 2: In Vivo Metabolism of Polyester Monomers and Production of PHA Under Aerobic Conditions

Each of the polyester monomers was tested separately in a minimum media as sole carbon source to validate if P. denitrificans can grow on this substrate and therefore metabolize it. Moreover, tests were done to validate which monomer is faster consumed by P. denitrificans.

To test the ability to grow on the substrate as the sole carbon source, batch cultivations were performed aerobically and at 34° C., in mineral salt medium (MSM) and the corresponding monomers as sole carbon source. The inoculum of 2% came from a cultivation in LB medium under the same conditions. The results in Table 2 confirm that P. denitrificans can degrade the monomers of 12 different plastic polymers used in e.g. packaging.

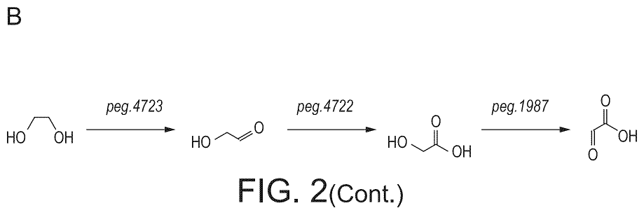

Kinetics analyses were obtained in batch cultivations performed in a 2L fermenter aerobically at 37° C. in in mineral salt medium (MSM) and the corresponding monomers as sole carbon source (0.3% w/v). Inoculum was 200 mL of a culture adapted to the same conditions at OD₆₀₀ of ˜0.1. Cultivations were run until maximum growth was reached (max OD₆₀₀ reading). Data presented is the result of triplicates. FIG. 3 shows the biomass formation (cell mass dry weight) of P. denitrificans when supplying different monomers as sole carbon source as in 0.3% (w/v) of the media. Table 3 shows that even polyester monomers which are commonly challenging for microbial processing such as 1,4 Butanediol have a significant yield.

TABLE 2
Ability of P. denitrificans to grow on different monomers as sole carbon source to
validate its metabolic potential to degrade and metabolize common plastic polymers.

| | Polymers | | | | | | | | | | | |
|---|---|---|---|---|---|---|---|---|---|---|---|---|
| Monomers | PBS | PBSA | PBST | PBSTIL | PBT | PBAT | PET | PEA | PLA | PCL | PHB | PHBV |
| Succinic acid | yes | yes | yes | yes | | | | | | | | |
| Lactic acid | | | | yes | | | | | yes | | | |
| Ethylene glycol | | | | | | | yes | yes | | | | |
| Adipic acid | | | | | | yes | | | | | | |
| 3-Hydroxybutyric acid | | | | | | | | | | | yes | |
| 6-Hydroxycaproic acid | | | | | | | | | | yes | | |
| 3-Hydroxyvaleric acid | | | | | | | | | | | | yes |
| 1,4-Butanediol | yes | yes | yes | yes | yes | yes | | | | | | |

TABLE 3
Kinetics parameters for assimilation of different monomers
of plastic polymers as sole carbon source by P. denitrificans
when maximal growth reached.

| | | μ cell | Y cell |
|---|---|---|---|
| | % substrate | growth rate | biomass (g |
| Monomers | consumed | (g · h⁻¹) | biomass · mol⁻¹) |
| Succinic acid | 98.6 | 0.232 ± 0.002 | 48.4 ± 2.12 |
| Lactic acid | 100.0 | 0.215 ± 0.008 | 42.9 ± 1.77 |
| Ethylene glycol | 100.0 | 0.045 ± 0.001 | 29.9 ± 0.07 |
| Adipic acid | 81.5 | 0.037 ± 0.001 | 62.8 ± 3.61 |
| 3-Hydroxybutyric acid | 100.0 | 0.130 ± 0.002 | 42.4 ± 1.61 |
| 6-Hydroxycaproic acid | 92.5 | 0.034 ± 0.001 | 52.7 ± 1.06 |
| 3-Hydroxyvaleric acid | 100.0 | 0.041 ± 0.001 | 37.9 ± 2.05 |
| 1,4-Butanediol | 80.6 | 0.022 ± 0.001 | 41.6 ± 1.77 |

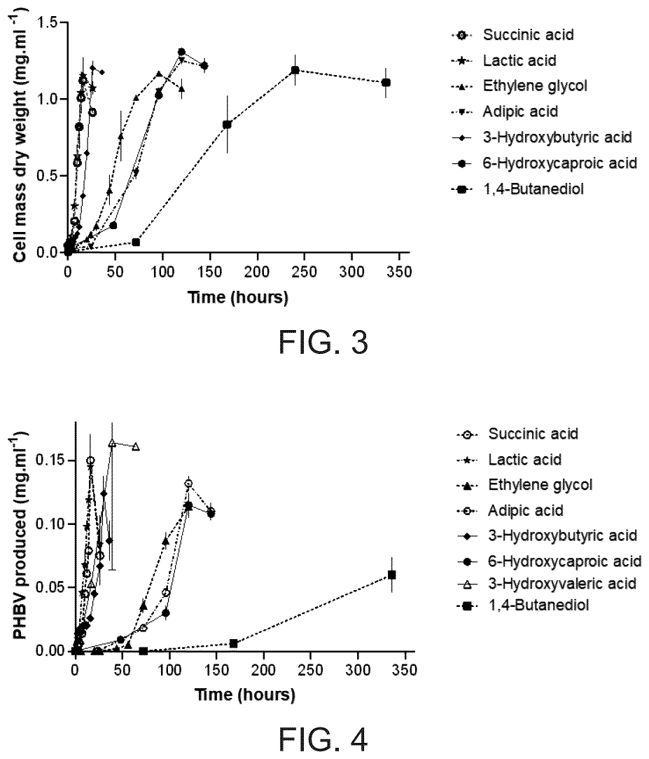

The microbial broth resulting was analyzed by GC-MS, which confirmed the ability of P. denitrificans to produce PHA polymer from a wide array of polyester monomers as substrates. FIG. 4 and Table 4 shows that P. denitrificans produces polymers with different ratio of the different components of PHA polymer depending on the monomer used as substrate. The ratios of the types of PHA vary as well.

TABLE 4
Bio-polymer production by P. denitrificans. Yield in
relation to biomass (Y_{PHA}), ratio of the different components
of PHA polymer (PHBV), % of 3HB or 3HV are shown.

| | Y | | |
|---|---|---|---|
| | (μg PHA | 3HB/PHA | 3HV/PHA |
| Monomers | mg⁻¹ DW) | (%) | (%) |
| Succinic acid | 100.7 ± 5.4 | 92.9% (±0.8) | 7.1% (±0.8) |
| Lactic acid | 113.6 ± 12.0 | 95.4% (±0.2) | 4.6% (±0.2) |
| Ethylene glycol | 73.0 ± 1.7 | 92.3% (±0.4) | 7.7% (±0.4) |
| Adipic acid | 80.5 ± 4.1 | 93.2% (±0.9) | 6.8% (±0.9) |
| 3-Hydroxybutyric acid | 68.3 ± 13.8 | 92.2% (±0.7) | 7.8% (±0.7) |
| 6-Hydroxycaproic acid | 79.6 ± 2.3 | 91.8% (±0.3) | 8.2% (±0.3) |
| 3-Hydroxyvaleric acid | 175.5 ± 12.0 | 6.6% (±1.9) | 93.4% (±1.9) |
| 1,4-Butanediol | 53.6 ± 9.8 | 90.9% (±3.1) | 9.1% (±3.1) |

### Example 3: In Vivo Metabolism of Polyester Monomers and Production of PHA Under Anoxic Conditions

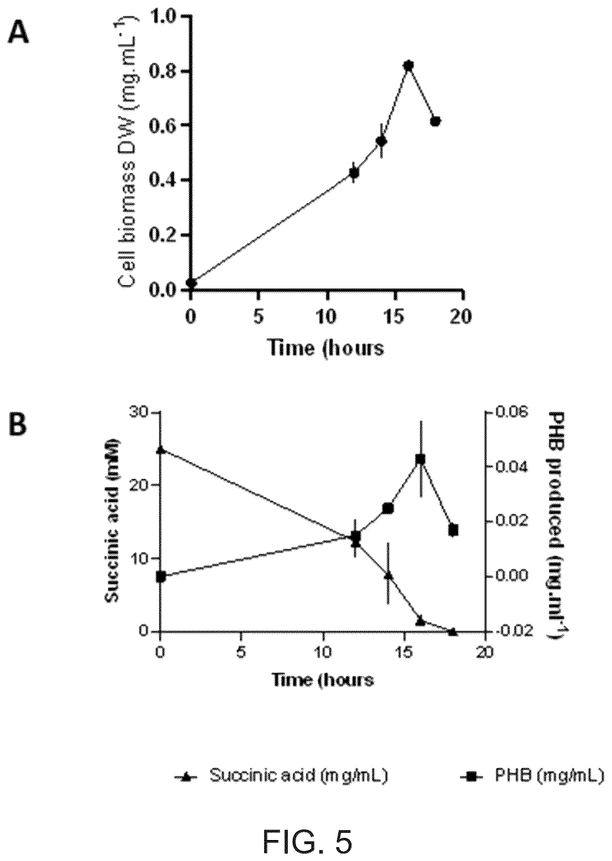

Batch cultivations were performed anoxically at 34° C., in mineral salt medium (MSM) and succinic acid as sole carbon source (monomer of PBS, PBSA, PBST, PBSTIL). The inoculum of 2% came from a cultivation in LB medium under the same conditions. FIG. 5 and Table 5 shows that P. denitrificans was capable of utilising polyester monomer to produce PHA under anoxic conditions

TABLE 5
Kinetics parameters for assimilation of different monomers
of plastic polymers as sole carbon source by P. denitrificans
when maximal growth reached under anoxic conditions.

| | % substrate | μ cell growth rate | Y cell biomass (g |
|---|---|---|---|
| Monomers | consumed | (g · h⁻¹) | biomass · mol⁻¹) |
| Succinic acid | 100 | 0.083 ± 0.001 | 0.28 |

### Example 4: In Vivo Metabolism of Polyester Polymers and Production of PHA

Biological polymer recycling by P. denitrificans may also be achieved on polymers, not only on the single monomers. In waste management, polymers (e.g., from packaging) arrive to waste treatment centers whole. A mechanical (e.g., shredding) and/or chemical treatment (e.g., alkaline treatment), promotes degradation of the complex compound into smaller particles and makes the polymer monomers accessible for recycling. Bio-recycling by P. denitrificans may also be achieved from the pretreated polymers and generates new biopolymer (e.g., PHA).

Polymers were shredded (500-1000 μm) and chemically treated with an alkaline solution (2M NaOH), incubated at 37° C. while constantly stirred (300 rpm) for 7 days. The polymer tested were PHB, PLA, PHBV, PHBH, PLA/PCL. The alkali pretreated polymer was neutralized (with HCL to pH 7) and 10% (v/v) was added to mineral salt medium (90% (v/v) as sole carbon source. The culture bottles were inoculated with 1 mL of fresh P. denitrificans culture and incubated for 3 days at 30° C. under orbital shaking a 300 rpm. Biomass growth (estimated as optical density) and PHB content were periodically monitored.

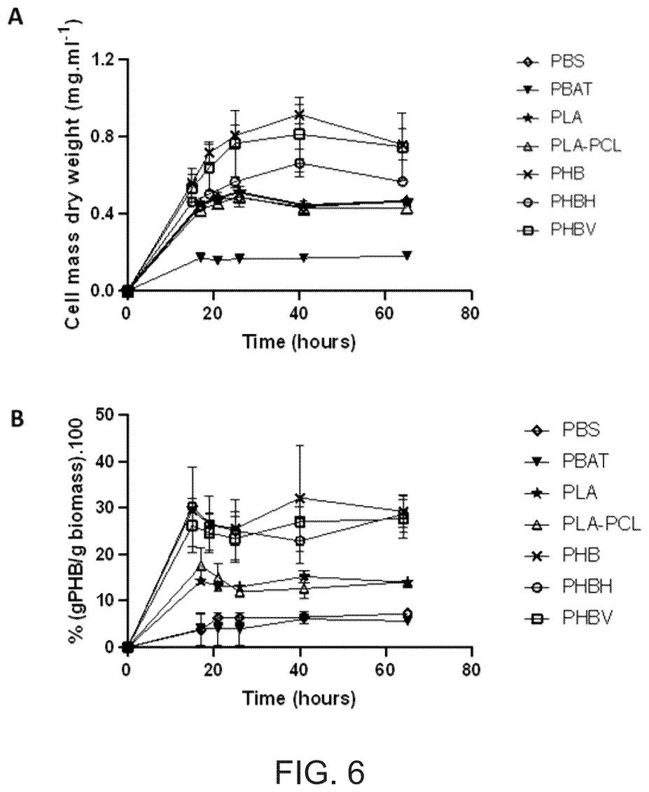

FIG. 6 shows that the bacterium grows on all pretreated polymers as sole carbon source and produced PHB. When PHB, PHBH or PHBV hydrolysates were used as substrates, PHB up to 30% of the microbial cell mass was accumulated. With PLA and PCL/PLA blends, the accumulated PHB was 15%. With PBS and PBAT hydrolysates accumulation was around 6%.

### Embodiments

Various preferred features and embodiments of the present invention will now be described with reference to the following numbered paragraphs (paras).
1. A method for producing polyhydroxyalkanoate (PHA) from polyester waste, the method comprising the steps of: (a) providing a culture broth comprising polyester waste; and (b) cultivating a microbe in the culture broth to produce PHA, wherein the microbe utilises a plurality of polyester monomers from the polyester waste to produce the PHA.
2. A method for producing polyhydroxyalkanoate (PHA) from polyester waste, the method comprising the steps of: (a) providing a culture broth comprising polyester waste; and (b) cultivating a microbe in the culture broth to produce PHA, wherein the microbe utilises 1,4-butanediol from the polyester waste to produce the PHA.
3. The method according to para 1 or 2, wherein the microbe is from the genus Paracoccus.
4. The method according to any preceding para, wherein the microbe is a Paracoccus denitrificans.
5. The method according to any preceding para, wherein the microbe is Paracoccus denitrificans DSM 413, or a derivative thereof.
6. The method according to any preceding para, wherein the microbe is Paracoccus denitrificans DSM 413, Paracoccus denitrificans PD1222, Paracoccus denitrificans CNCM I-5881, Paracoccus denitrificans ATCC 19367, Paracoccus denitrificans ATCC 17741, Paracoccus denitrificans ATCC 13543, Paracoccus denitrificans NCIB 8944, Paracoccus denitrificans NRRL B-3785, Paracoccus denitrificans CCM 982, Paracoccus denitrificans LMD 22.21, Paracoccus denitrificans JCM 21484, Paracoccus denitrificans NBRC 102528, Paracoccus denitrificans NCCB 22021, Paracoccus denitrificans NBRC 13301, Paracoccus denitrificans NCIMB 8944, Paracoccus denitrificans DSM 15418, Paracoccus denitrificans DSM 415, Paracoccus denitrificans NCIMB 11627, Paracoccus denitrificans NCIMB 9722, Paracoccus denitrificans IMET 10380, Paracoccus denitrificans VKM B-1324, or Paracoccus denitrificans ICPB 3979.
7. The method according to any preceding para, wherein the microbe comprises genes encoding for two or more pathways, three or more pathways, four or more pathways, five or more pathways, six or more pathways, or seven or more pathways selected from: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol.
8. The method according to any preceding para, wherein the microbe comprises genes encoding for each of: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol.
9. The method according to any preceding para, wherein the polyester waste comprises 1,4-butanediol.
10. The method according to any preceding para, wherein the polyester waste comprises two or more, three or more, four or more, five or more, six or more, or seven or more polyester monomers selected from succinic acid, lactic acid, ethylene glycol, adipic acid, 6-hydroxycaproic acid, 3-hydroxybutyric acid, 3-hydroxyvaleric acid, and 1,4-butanediol.
11. The method according to any preceding para, wherein the polyester waste comprises succinic acid, lactic acid, ethylene glycol, adipic acid, 6-hydroxycaproic acid, 3-hydroxybutyric acid, 3-hydroxyvaleric acid, and 1,4-butanediol.
12. The method according to any preceding para, wherein the polyester waste comprises the polyester monomers in the form of free monomers.
13. The method according to any preceding para, wherein the polyester waste comprises one or more, two or more, three or more, four or more, five or more, six or more, seven or more, or eight or more polyesters selected from: polybutylene succinate (PBS), polybutylene succinate adipate (PBSA), poly(butylene succinate-co-terephthalate) (PBST), poly(butylene succinate/terephthalate/isophthalate)-co-(lactate) (PBSTIL), polybutylene terephthalate (PBT), polybutylene adipate terephthalate (PBAT), polyethylene terephthalate (PET), poly(ethylene adipate) (PEA), polylactic acid (PLA), polycaprolactone (PCL), polyhydroxybutyrate (PHB), and poly(3-hydroxybutyrate-co-3-hydroxyvalerate) (PHBV).
14. The method according to any preceding para, wherein the polyester waste is pre-treated, optionally wherein the polyester waste is mechanically treated and/or chemically treated.
15. The method according to any preceding para, wherein the method further comprises a step of pre-treating the polyester waste.
16. The method according to any preceding para, wherein the method further comprises a step of mechanically treating the polyester waste.
17. The method according to para 16, wherein the polyester waste is shredded, optionally wherein the polyester waste is shredded to a particle size of from about 100 μm to about 5000 μm, from about 200 μm to about 4000 μm, from about 300 μm to about 3000 μm, from about 400 μm to about 2000 μm, or from about 500 μm to about 1000 μm.
18. The method according to any preceding para, wherein the method further comprises a step of chemically treating the polyester waste.
19. The method according to any preceding para, wherein the polyester waste undergoes alkaline treatment.
20. The method according to para 19, wherein the polyester waste is neutralised following the alkaline treatment.
21. The method according to any preceding para, wherein the culture broth comprises the polyester waste in an amount of from about 1 g/L to about 100 g/L, from about 1 g/L to about 50 g/L, from about 1 g/L to about 20 g/L, from about 2 g/L to about 10 g/L, or from about 2 g/L to about 5 g/L.
22. The method according to any preceding para, wherein the culture broth comprises mineral salt medium.
23. The method according to any preceding para, wherein the microbe is cultivated under aerobic or anoxic conditions.
24. The method according to any preceding para, wherein the microbe is cultivated under anoxic conditions.
25. The method according to any preceding para, wherein the microbe is cultivated for from about one to about seven days, from about two to about six days, or from about three to about five days.
26. The method according to any preceding para, wherein a single microbial strain is cultivated.
27. The method according to any preceding para, wherein the method comprises a single cultivation step.
28. The method according to any preceding para, wherein the microbe utilises at least three, at least four, at least five, at least six, at least seven, or at least eight polyester monomers from the polyester waste to produce the PHA.
29. The method according to any preceding para, wherein the microbe utilises polyester monomers from a plurality of polyesters from the polyester waste to produce the PHA, optionally wherein the microbe utilises polyester monomers from at least three, at least four, at least five, at least six, at least seven, or at least eight polyesters from the polyester waste to produce the PHA.
30. The method according to any preceding para, wherein at least about 50 wt %, at least about 60 wt %, at least about 70 wt %, at least about 75 wt %, or at least about 80 wt % of the polyester waste is utilised during the cultivation.
31. The method according to any preceding para, wherein at least about 0.01 mg/ml, at least about 0.02 mg/ml, at least about 0.03 mg/ml, at least about 0.04 mg/ml, at least about 0.05 mg/ml, or at least about 0.1 mg/ml PHA is produced.
32. The method according to any preceding para, wherein at least about 10 μg PHA/mg dry cell weight (DCW), at least about 20 μg PHA/mg DCW, at least about 30 μg PHA/mg DCW, at least about 40 μg PHA/mg DCW, or at least about 50 μg PHA/mg DCW is produced.
33. The method according to any preceding para, wherein the PHA comprises or consists of polyhydroxybutyrate (PHB) or a co-polymer thereof and/or polyhydroxyvalerate (PHV) or a co-polymer thereof.
34. The method according to any preceding para, wherein the PHA comprises or consists of polyhydroxybutyrate (PHB) or a co-polymer thereof.
35. The method according to any preceding para, wherein the PHA comprises or consists of poly(3-hydroxybutyrate-co-3-hydroxyvalerate) (PHBV).
36. The method according to any preceding para, wherein the method further comprises a step of recovering the PHA.
37. A culture broth comprising polyester waste and a microbe, wherein the microbe is capable of utilising a plurality of polyester monomers from the polyester waste to produce PHA.
38. The culture broth according to para 37, wherein the microbe is as defined according to any of paras 3 to 8, wherein the polyester waste is as defined according to any of paras 9 to 14, and/or wherein the culture broth is as defined according to para 21 or 22.
39. The culture broth according to para 37 or 38, wherein the culture broth further comprises PHA, optionally wherein the PHA is as defined according to any of paras 33 to 35.
40. A polyhydroxyalkanoate (PHA) produced by the method according to any of paras 1 to 39.
41. An article comprising or consisting of the PHA according to para 40.
42. The article according to para 41, wherein the article is packaging.
43. Use of a microbe for producing polyhydroxyalkanoate (PHA) from polyester waste, wherein the microbe utilises a plurality of polyester monomers from the polyester waste to produce the PHA.
44. Use of a microbe for producing polyhydroxyalkanoate (PHA) from polyester waste, wherein the microbe utilises 1,4-butanediol from the polyester waste to produce the PHA.
45. The use according to para 44 or 45, wherein the microbe is as defined according to any of paras 3 to 8, and/or wherein the polyester waste is as defined according to any of paras 9 to 14.
46. The use according to any of paras 43 to 45, wherein the microbe utilises at least three, at least four, at least five, at least six, at least seven, or at least eight polyester monomers from the polyester waste to produce the PHA.
47. The use according to any of paras 43 to 46, wherein the microbe utilises polyester monomers from a plurality of polyesters from the polyester waste to produce the PHA, optionally wherein the microbe utilises polyester monomers from at least three, at least four, at least five, at least six, at least seven, or at least eight polyesters from the polyester waste to produce the PHA.
48. The use according to any of paras 43 to 47, wherein the PHA comprises polyhydroxybutyrate (PHB) or a co-polymer thereof and/or polyhydroxyvalerate (PHV) or a co-polymer thereof.
49. The use according to any of paras 43 to 48, wherein the PHA comprises or consists of poly(3-hydroxybutyrate-co-3-hydroxyvalerate) (PHBV).
50. The use according to any of paras 43 to 49, wherein the microbe produces the PHA under anoxic conditions.
51. The use according to any of paras 43 to 50, wherein the microbe produces the PHA in a single cultivation step.
52. A microbe for producing polyhydroxyalkanoate (PHA) from polyester waste, the microbe comprising genes encoding pathways for the utilisation of a plurality of polyester monomers and for the synthesis of PHA, wherein the microbe has been genetically engineered to express at least part of one or more of the pathways.
53. The microbe according to para 52, wherein the microbe comprises genes encoding for one or more, two or more, three or more, four or more, five or more, six or more, or seven or more pathways selected from: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol.
54. The microbe according to para 52 or 53, wherein the microbe comprises genes encoding for: (i) a pathway for the utilisation of succinic acid; (ii) a pathway for the utilisation of lactic acid; (iii) a pathway for the utilisation of ethylene glycol; (iv) a pathway for the utilisation of adipic acid; (v) a pathway for the utilisation of 6-hydroxycaproic acid; (vi) a pathway for the utilisation of 3-hydroxybutyric acid; (vii) a pathway for the utilisation of 3-hydroxyvaleric acid; and (viii) a pathway for the utilisation of 1,4-butanediol.
55. The microbe according to any of paras 52 to 54, wherein the microbe has been genetically engineered to express at least part of a pathway for the utilisation of succinic acid, optionally wherein the microbe has been genetically engineered to express one or more gene encoding a succinate dehydrogenase.
56. The microbe according to any of paras 52 to 55, wherein the microbe has been genetically engineered to express a succinate dehydrogenase having at least 70% sequence identity to the amino acid sequence of SEQ ID NO: 1.
57. The microbe according to any of paras 52 to 56, wherein the microbe has been genetically engineered to introduce a gene encoding a succinate dehydrogenase and having at least 70% sequence identity to the nucleotide sequence of SEQ ID NO: 2.
58. The microbe according to any of paras 52 to 57, wherein the microbe has been genetically engineered to express at least part of a pathway for the utilisation of lactic acid, optionally wherein the microbe has been genetically engineered to express one or more gene encoding a D-lactate dehydrogenase.
59. The microbe according to any of paras 52 to 58, wherein the microbe has been genetically engineered to express a D-lactate dehydrogenase having at least 70% sequence identity to the amino acid sequence of SEQ ID NO: 3.
60. The microbe according to any of paras 52 to 59, wherein the microbe has been genetically engineered to introduce a gene encoding a D-lactate dehydrogenase and having at least 70% sequence identity to the nucleotide sequence of SEQ ID NO: 4.
61. The microbe according to any of paras 52 to 60, wherein the microbe has been genetically engineered to express at least part of a pathway for the utilisation of ethylene glycol, optionally wherein the microbe has been genetically engineered to express one or more gene encoding an alcohol dehydrogenase, an aldehyde dehydrogenase, and/or a glyoxylate reductase.
62. The microbe according to any of paras 52 to 61, wherein the microbe has been genetically engineered to express one or more of: (i) an alcohol dehydrogenase having at least 70% sequence identity to the amino acid sequence of SEQ ID NO: 5; (ii) an aldehyde dehydrogenase having at least 70% sequence identity to the amino acid sequence of SEQ ID NO: 7; and (iii) a glyoxylate reductase having at least 70% sequence identity to the amino acid sequence of SEQ ID NO: 9.
63. The microbe according to any of paras 52 to 62, wherein the microbe has been genetically engineered to introduce one or more of: (i) a gene encoding an alcohol dehydrogenase and having at least 70% sequence identity to the nucleotide sequence of SEQ ID NO: 6; (ii) a gene encoding an aldehyde dehydrogenase and having at least 70% sequence identity to the nucleotide sequence of SEQ ID NO: 8; and (iii) a gene encoding a glyoxylate reductase and having at least 70% sequence identity to the nucleotide sequence of SEQ ID NO: 10.
64. The microbe according to any of paras 52 to 63, wherein the microbe has been genetically engineered to express at least part of a pathway for the utilisation of adipic acid, optionally wherein the microbe has been genetically engineered to express one or more gene encoding a long-chain-fatty-acid-CoA ligase, an acyl-CoA dehydrogenase, an enoyl-CoA hydratase, a 3-hydroxybutyryl-CoA dehydrogenase, and/or a 3-oxoadipyl-CoA thiolase.
65. The microbe according to any of paras 52 to 64, wherein the microbe has been genetically engineered to express one or more of: (i) a long-chain-fatty-acid-CoA ligase having at least 70% sequence identity to the amino acid sequence of SEQ ID NO: 11; (ii) an acyl-CoA dehydrogenase having at least 70% sequence identity to the amino acid sequence of SEQ ID NO: 13; (iii) an enoyl-CoA hydratase having at least 70% sequence identity to the amino acid sequence of SEQ ID NO: 15; (iv) a 3-hydroxybutyryl-CoA dehydrogenase having at least 70% sequence identity to the amino acid sequence of SEQ ID NO: 17; and (v) a 3-oxoadipyl-CoA thiolase having at least 70% sequence identity to the amino acid sequence of SEQ ID NO: 19.
66. The microbe according to any of paras 52 to 65, wherein the microbe has been genetically engineered to introduce one or more of: (i) a gene encoding a long-chain-fatty-acid-CoA ligase and having at least 70% sequence identity to the nucleotide sequence of SEQ ID NO: 12; (ii) a gene encoding an acyl-CoA dehydrogenase and having at least 70% sequence identity to the nucleotide sequence of SEQ ID NO: 14; (iii) a gene encoding an enoyl-CoA hydratase and having at least 70% sequence identity to the nucleotide sequence of SEQ ID NO: 16; (iv) a gene encoding a 3-hydroxybutyryl-CoA dehydrogenase and having at least 70% sequence identity to the nucleotide sequence of SEQ ID NO: 18; and (v) a gene encoding a 3-oxoadipyl-CoA thiolase and having at least 70% sequence identity to the nucleotide sequence of SEQ ID NO: 20.
67. The microbe according to any of paras 52 to 66, wherein the microbe has been genetically engineered to express at least part of a pathway for the utilisation of 6-hydroxycaproic acid, optionally wherein the microbe has been genetically engineered to express one or more gene encoding an alcohol dehydrogenase and/or an aldehyde dehydrogenase.
68. The microbe according to any of paras 52 to 67, wherein the microbe has been genetically engineered to express: (i) an alcohol dehydrogenase having at least 70% sequence identity to the amino acid sequence of SEQ ID NO: 21; and/or (ii) an aldehyde dehydrogenase having at least 70% sequence identity to the amino acid sequence of SEQ ID NO: 23.
69. The microbe according to any of paras 52 to 68, wherein the microbe has been genetically engineered to introduce: (i) a gene encoding an alcohol dehydrogenase and having at least 70% sequence identity to the nucleotide sequence of SEQ ID NO: 22; and/or (ii) a gene encoding an aldehyde dehydrogenase and having at least 70% sequence identity to the nucleotide sequence of SEQ ID NO: 24.
70. The microbe according to any of paras 52 to 69, wherein the microbe has been genetically engineered to express at least part of a pathway for the utilisation of 3-hydroxybutyric acid, optionally wherein the microbe has been genetically engineered to express one or more gene encoding an Acyl-CoA synthetase, and/or a 3-hydroxybutyrate dehydrogenase.
71. The microbe according to any of paras 52 to 70, wherein the microbe has been genetically engineered to express: (i) an Acyl-CoA synthetase having at least 70% sequence identity to the amino acid sequence of SEQ ID NO: 25; and/or (ii) a a 3-hydroxybutyrate dehydrogenase having at least 70% sequence identity to the amino acid sequence of SEQ ID NO: 27.
72. The microbe according to any of paras 52 to 71, wherein the microbe has been genetically engineered to introduce: (i) a gene encoding an Acyl-CoA synthetase and having at least 70% sequence identity to the nucleotide sequence of SEQ ID NO: 26; and/or (ii) a gene encoding a 3-hydroxybutyrate dehydrogenase and having at least 70% sequence identity to the nucleotide sequence of SEQ ID NO: 28.
73. The microbe according to any of paras 52 to 72, wherein the microbe has been genetically engineered to express at least part of a pathway for the utilisation of 3-hydroxyvaleric acid, optionally wherein the microbe has been genetically engineered to express one or more gene encoding an Acyl-CoA synthetase, a 3-hydroxybutyrate dehydrogenase, and/or a 3-ketoacyl-CoA thiolase.
74. The microbe according to any of paras 52 to 73, wherein the microbe has been genetically engineered to express one or more of: (i) an Acyl-CoA synthetase having at least 70% sequence identity to the amino acid sequence of SEQ ID NO: 25; (ii) a 3-hydroxybutyrate dehydrogenase having at least 70% sequence identity to the amino acid sequence of SEQ ID NO: 27; and (iii) a 3-ketoacyl-CoA thiolase having at least 70% sequence identity to the amino acid sequence of SEQ ID NO: 29.
75. The microbe according to any of paras 52 to 74, wherein the microbe has been genetically engineered to introduce one or more of: (i) a gene encoding an Acyl-CoA synthetase and having at least 70% sequence identity to the nucleotide sequence of SEQ ID NO: 26; (ii) a gene encoding a 3-hydroxybutyrate dehydrogenase and having at least 70% sequence identity to the nucleotide sequence of SEQ ID NO: 28; and (iii) a gene encoding a 3-ketoacyl-CoA thiolase and having at least 70% sequence identity to the nucleotide sequence of SEQ ID NO: 30.
76. The microbe according to any of paras 52 to 75, wherein the microbe has been genetically engineered to express at least part of a pathway for the utilisation of 1,4-butanediol, optionally wherein the microbe has been genetically engineered to express one or more gene encoding a methanol dehydrogenase, an aldehyde dehydrogenase, an alcohol dehydrogenase, and/or a succinate-semialdehyde dehydrogenase.
77. The microbe according to any of paras 52 to 76, wherein the microbe has been genetically engineered to express one or more of: (i) a methanol dehydrogenase having at least 70% sequence identity to the amino acid sequence of SEQ ID NO: 31; (ii) a methanol dehydrogenase having at least 70% sequence identity to the amino acid sequence of SEQ ID NO: 33; (iii) an aldehyde dehydrogenase having at least 70% sequence identity to the amino acid sequence of SEQ ID NO: 23; (iv) an aldehyde dehydrogenase having at least 70% sequence identity to the amino acid sequence of SEQ ID NO: 35; (v) an alcohol dehydrogenase having at least 70% sequence identity to the amino acid sequence of SEQ ID NO: 37; (vi) an alcohol dehydrogenase having at least 70% sequence identity to the amino acid sequence of SEQ ID NO: 5; (vii) a succinate-semialdehyde dehydrogenase having at least 70% sequence identity to the amino acid sequence of SEQ ID NO: 39; (viii) a succinate-semialdehyde dehydrogenase having at least 70% sequence identity to the amino acid sequence of SEQ ID NO: 41; and (ix) a succinate-semialdehyde dehydrogenase having at least 70% sequence identity to the amino acid sequence of SEQ ID NO: 43.
78. The microbe according to any of paras 52 to 77, wherein the microbe has been genetically engineered to introduce one or more of: (i) a gene encoding a methanol dehydrogenase and having at least 70% sequence identity to the nucleotide sequence of SEQ ID NO: 32; (ii) a gene encoding a methanol dehydrogenase and having at least 70% sequence identity to the nucleotide sequence of SEQ ID NO: 34; (iii) a gene encoding an aldehyde dehydrogenase and having at least 70% sequence identity to the nucleotide sequence of SEQ ID NO: 24; (iv) a gene encoding an aldehyde dehydrogenase and having at least 70% sequence identity to the nucleotide sequence of SEQ ID NO: 36; (v) a gene encoding an alcohol dehydrogenase and having at least 70% sequence identity to the nucleotide sequence of SEQ ID NO: 38; (vi) a gene encoding an alcohol dehydrogenase and having at least 70% sequence identity to the nucleotide sequence of SEQ ID NO: 6; (vii) a gene encoding a succinate-semialdehyde dehydrogenase and having at least 70% sequence identity to the nucleotide sequence of SEQ ID NO: 40; (viii) a gene encoding a succinate-semialdehyde dehydrogenase and having at least 70% sequence identity to the nucleotide sequence of SEQ ID NO: 42; and (ix) a gene encoding a succinate-semialdehyde dehydrogenase and having at least 70% sequence identity to the nucleotide sequence of SEQ ID NO: 44.
79. The microbe according to any of paras 52 to 78, wherein the microbe has been genetically engineered to express at least part of a pathway for the synthesis of a PHA, optionally wherein the microbe has been genetically engineered to express one or more gene encoding a 3-ketoacyl-CoA thiolase, an enoyl-CoA hydratase, and/or a PHA synthase.
80. The microbe according to any of paras 52 to 79, wherein the microbe has been genetically engineered to express one or more of: (i) a 3-ketoacyl-CoA thiolase having at least 70% sequence identity to the amino acid sequence of SEQ ID NO: 45; (ii) an enoyl-CoA hydratase having at least 70% sequence identity to the amino acid sequence of SEQ ID NO: 47; and (iii) a PHA synthase having at least 70% sequence identity to the amino acid sequence of SEQ ID NO: 49.
81. The microbe according to any of paras 52 to 80, wherein the microbe has been genetically engineered to introduce one or more of: (i) a gene encoding a 3-ketoacyl-CoA thiolase and having at least 70% sequence identity to the nucleotide sequence of SEQ ID NO: 46; (ii) a gene encoding an enoyl-CoA hydratase and having at least 70% sequence identity to the nucleotide sequence of SEQ ID NO: 48; (iii) a gene encoding a PHA synthase and having at least 70% sequence identity to the nucleotide sequence of SEQ ID NO: 50.
82. The microbe according to any of paras 52 to 81, wherein the microbe has been genetically engineered by transfection, by transduction, or by gene-editing.
83. The microbe according to any of paras 52 to 82, wherein the microbe is a bacterium.
84. The microbe according to any of paras 52 to 83, wherein the microbe is capable of utilising one or more, two or more, three or more, four or more, five or more, six or more, or seven or more polyester monomers selected from: succinic acid, lactic acid, ethylene glycol, adipic acid, 6-hydroxycaproic acid, 3-hydroxybutyric acid, 3-hydroxyvaleric acid, and 1,4-butanediol to produce PHA.
85. The microbe according to any of paras 52 to 84, wherein the microbe is capable of utilising each of succinic acid, lactic acid, ethylene glycol, adipic acid, 6-hydroxycaproic acid, 3-hydroxybutyric acid, 3-hydroxyvaleric acid, and 1,4-butanediol to produce PHA.
86. A vector comprising a gene encoding an enzyme having at least 70% sequence identity to any of SEQ ID NOs: 1, 3, 5, 7, 9, 11, 13, 15, 17, 19, 21, 23, 25, 27, 29, 31, 33, 35, 37, 39, 41, 43, 45, 47 or 49.
87. A vector comprising a gene comprising or consisting of a nucleotide sequence having at least 70% sequence identity to any of SEQ ID NOs: 2, 4, 6, 8, 10, 12, 14, 16, 18, 20, 22, 24, 26, 28, 30, 32, 34, 36, 38, 40, 42, 44, 46, 48, 50.
88. The vector according to para 86 or 87, wherein the vector is a plasmid or a viral vector.
89. A cell comprising the vector according to any of paras 86 to 88. 90. The cell according to para 89, wherein the cell is a microbe, optionally wherein the cell is a bacterium.

The practice of the present invention will employ, unless otherwise indicated, conventional techniques of which are within the capabilities of a person of ordinary skill in the art. Such techniques are explained in the literature. It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A method for producing polyhydroxyalkanoate (PHA) from polyester waste, the method comprising the steps of:
(a) providing a culture broth comprising polyester waste; and
(b) cultivating a microbe in the culture broth to produce PHA, wherein the microbe utilises a plurality of polyester monomers from the polyester waste to produce the PHA.

2. The method according to claim 1, wherein the microbe is from the genus Paracoccus, optionally wherein the microbe is a Paracoccus denitrificans.

3. The method according to claim 1, wherein the microbe is Paracoccus denitrificans DSM 413, or a derivative thereof, optionally wherein the microbe is Paracoccus denitrificans DSM 413, Paracoccus denitrificans PD1222, Paracoccus denitrificans CNCM 1-5881, Paracoccus denitrificans ATCC 19367, Paracoccus denitrificans ATCC 17741, Paracoccus denitrificans ATCC 13543, Paracoccus denitrificans NCIB 8944, Paracoccus denitrificans NRRL B-3785, Paracoccus denitrificans CCM 982, Paracoccus denitrificans LMD 22.21, Paracoccus denitrificans JCM 21484, Paracoccus denitrificans NBRC 102528, or Paracoccus denitrificans NCCB 22021.

4. The method according to claim 1, wherein the polyester waste comprises two or more polyester monomers selected from the group consisting of succinic acid, lactic acid, ethylene glycol, adipic acid, 6-hydroxycaproic acid, 3-hydroxybutyric acid, 3-hydroxyvaleric acid, and 1,4-butanediol.

5. The method according to claim 1, wherein the polyester waste is pre-treated, optionally wherein the polyester waste is subjected to one or both of mechanical treatment and chemical treatment.

6. The method according to claim 1, wherein the microbe is cultivated under anoxic conditions, optionally wherein a single microbial strain is cultivated and/or wherein the method comprises a single cultivation step.

7. The method according to claim 1, wherein at least about 50 wt % of the polyester waste is utilised during the cultivation.

8. The method according to claim 1, wherein the PHA comprises polyhydroxybutyrate (PHB), or a co-polymer thereof, polyhydroxyvalerate (PHV), or a co-polymer thereof, or combinations thereof, optionally wherein the PHA comprises poly(3-hydroxybutyrate-co-3-hydroxyvalerate) (PHBV).

9. A culture broth comprising polyester waste and a microbe, wherein the microbe is capable of utilising a plurality of polyester monomers from the polyester waste to produce PHA.

10. The PHA produced by the method according to claim 1.

11. An article comprising the PHA according to claim 10.

12. (canceled)

13. A microbe for producing polyhydroxyalkanoate (PHA) from polyester waste, the microbe comprising genes encoding pathways for the utilisation of a plurality of polyester monomers and for the synthesis of PHA, wherein the microbe has been genetically engineered to express at least part of one or more of the pathways.

14. A vector comprising: a gene encoding an enzyme having at least 70% sequence identity to any of SEQ ID NOs: 1, 3, 5, 7, 9, 11, 13, 15, 17, 19, 21, 23, 25, 27, 29, 31, 33, 35, 37, 39, 41, 43, 45, 47 or 49; a gene comprising a nucleotide sequence having at least 70% sequence identity to any of SEQ ID NOs: 2, 4, 6, 8, 10, 12, 14, 16, 18, 20, 22, 24, 26, 28, 30, 32, 34, 36, 38, 40, 42, 44, 46, 48, 50, or combinations thereof.

15. A cell comprising the vector according to claim 14, optionally wherein the cell is a microbe, optionally wherein the cell is a bacterium.


SEQUENCE LISTING (50 sequences)

SEQ ID NO: 1 - protein, 569 aa, Paracoccus denitrificans
MPKPDLPDLPDTCDLLVVGSGAAGMAAAISAHHHGLKPVIVEKSEFFGGSTAVSGGAIWVPCNPIAAAAGMTDDREAARAYIRGETGNRFNAELVDAFLDKSPEAIGFFHERTALKMAHRALSPDYHSDAPGATEGGRALDALDYDGRRLGADLYRMRPPIADFTILGGMPLGRPDIFHFLRMTRSVKSAAYATGAVLRYFRDRLTWGRNTRLVMGAAVSGRLAETVFARNIPLFTGHELVRLLQDESGRVVGAELKGPRGVCRIAAHRGVVLAAGGYPHDAARRAQSFEHVRRGLPHYSMSPVSGTGGGIAAAEAVGAAFVDTNPNAGFWTPVSLLRNADGSVRPFPHLFLDRAKPGVIAVGHDGRRFVNEASSYHDFVQGLIAKLLADGEKSAWLVADHRAMRRYGLGAAHAFPARIGRHVASGYLKRDATLEGLARQCGIDVATFRQTVALFNEAAARGEDPAFGKGSTSYQRYLGDGENRPNPCLRPLEGPFYAVEIYPGDIGTSMGLDITAKGEVRDSRGRTIPGLYACGNDINSVMSGAYPGPGITLGPALTFGYVIGQSAAA

SEQ ID NO: 2 - DNA, 1710 bp, Paracoccus denitrificans
  source 1..1710: mol_type = other DNA
atgccgaagcctgatctgccggacctgcccgatacctgcgaccttctggtcgtgggttcgggtgccgcgggcatggcggcggcgatctctgcccatcatcacgggttgaagccggtcatcgtcgagaagtccgaattcttcggcggctccaccgccgtttccggcggcgcgatctgggtgccgtgcaatccgatcgccgcggcggccggcatgaccgacgaccgcgaggcggcgcgcgcctatatccggggcgagacgggcaatcgcttcaatgccgagctggtggacgcctttctcgacaagagccccgaggccatcggctttttccacgaaagaaccgcgctcaagatggcgcatcgcgcgctgtcgcccgactatcattcggatgcgccgggtgcgaccgaaggcggccgggcgctggacgcgctggattatgacggccgccggctgggggcggacctttatcggatgcgcccgcccatcgccgatttcacgatcctcggcggaatgccgctggggcgcccggacatctttcacttcctgcgcatgacccggtcggtgaaatcggcggcctatgcgaccggggcagtgctgcgctatttccgcgaccggctgacctgggggcggaacacgcggctggtgatgggcgcggcggtctcggggcgcctggccgagacggtgttcgcaaggaacatcccgctttttaccgggcatgagctggtgcggctgctgcaggacgaatccgggcgggtcgtcggggccgaactcaaggggccacggggcgtttgccggatcgcggcgcatcgcggcgtggttcttgcggcgggcggctatccgcatgatgcggcgcgtcgggcgcaaagcttcgagcatgtcaggcgcggcctgccgcattattcgatgtcgccggtctcgggcaccggcggcgggatcgccgcggccgaggccgtcggagcggcctttgtcgacaccaaccccaatgccggtttctggacgccggtttcgctcttgcgcaatgccgacggatcggtgcggcccttcccgcatcttttcctggaccgtgccaagcccggcgtgatcgccgtcggccatgacggccggcgcttcgtcaacgaggcttccagctatcatgacttcgtgcagggattgatcgcgaagctgctggccgacggagagaaatccgcctggctggtcgccgatcaccgggcgatgcggcgctatgggctgggtgccgcgcatgcctttccggcgcggatcggccggcatgtcgccagcggctatctgaagcgcgacgcgacgctggaggggctggcccggcaatgcggcatcgacgtcgcgacgttccggcagacggtcgccctgttcaacgaggcggcggcacgcggcgaagacccggccttcggcaagggatcgaccagctatcagcgctatctgggcgatggcgagaaccggccgaacccctgcctgcgcccgctggaggggccgttctacgccgtcgagatctatccgggcgacatcggcacctctatgggcctggacatcacggcgaaaggcgaggtcagggacagccggggcaggacgatacccggcctctatgcctgcggcaacgacatcaattcggtgatgtccggcgcctatcccgggccgggcatcacgctgggtccggcgctgaccttcggatatgtcatcgggcaatcggcggccgcatga

SEQ ID NO: 3 - protein, 569 aa, Paracoccus denitrificans
MTRPGQAEAAADRTALLDRLRRIVGPAHVLTADRATRRYTRGFRYGEGPVAAVVRPGSLVQMWRVLNAAVASGRAVILQAANTGLTGGSTPWGQDYDREIVLVSVMRLRGIHLIGAGEQVLCLPGATLDALEKRLRPLGREPHSVIGSSCIGASVLGGICNNSGGALIQRGPAYTEMSLYAEVREDGSVALVNHLGLDLGDDPEEILARVEAGELPAPAPTDAWASDREYADHVRDIEAETPARFNADPRRLHESSGCAGKLAVFAVRLDTFQAEKDTAVFYVGSNDPDELTEIRRHILAHFQSLPIAGEYIHREAYDIAAKYGKDTFLFIRHAGTDRMPAFFAAKARMDALTERLGLGATLSDRLAQGVAALMPQHLPRRMNDFRDRYEHHLLLRMGGAGIAEARDYLGAIFPSASGAMFECTPDEGKAAFLHRFAVAGAAVRYRAIHAREVQDIVALDIALRRNDRDWVERLPPDLDAKLEKKLYYGHFFCHVFHQDYVVKKGQDCLAVEHEMWRLLDRRGAEYPAEHNVGHLYHAKPELAGFYRQLDPTNSLNPGIGQTSKCAHWH

SEQ ID NO: 4 - DNA, 1710 bp, Paracoccus denitrificans
  source 1..1710: mol_type = other DNA
atgacgcgacccggccaggccgaggcagccgcagaccgcaccgcattgctggaccggctgcgccggatcgtgggaccggcgcatgtgctgaccgccgaccgcgccacccgccgctatacccgcggcttccgctatggcgaggggccggtggccgcggtggtccgccccggctctctggtccagatgtggcgggtcctgaacgccgccgtcgcctcggggcgcgcggtgatcctgcaggccgcgaataccgggctgaccggcggctcgaccccctggggccaggattacgaccgcgagatcgtgctggtctcggtcatgcgcctgcgcggcatccacctcatcggcgcgggcgaacaggtgctctgcctgcccggcgccacgctcgacgcgctggaaaagcgcctgcgccccctgggacgagagccgcattcggtcatcggctcgtcctgcatcggcgcctctgtcctgggcggcatctgcaacaattccggcggcgcgctgatccagcgcggcccggcctataccgagatgagcctttacgccgaggtgcgcgaggacggctcggtggcgctggtcaaccatctgggcctcgacctcggcgacgatcccgaggaaatcctggcccgcgtcgaggcgggcgaattgcccgcccccgcgcccaccgacgcctgggcctcggaccgcgaatatgccgaccatgtccgcgacatcgaggccgagaccccggcccgcttcaacgccgatccgcgccggctgcacgaatcctcgggctgcgccggcaagctcgcagtctttgccgtgcggctggacacgttccaggccgaaaaggacacggcggtcttctatgtcggcagcaacgacccggacgagttgaccgagatccgccgccatatcctggcgcatttccagagcctgcccatcgccggcgaatacatccaccgcgaagcctatgacatcgcagcgaaatacggcaaggacaccttcctgttcatccgccatgccggcaccgaccgcatgccggcattcttcgccgccaaggcgcgcatggacgcgctgaccgagcggctggggctgggcgccaccctctcggaccggctggcgcagggcgtcgcggcgctgatgccccagcacctgcccaggcgcatgaacgatttccgcgaccgctacgaacaccacctgctgctgcgcatgggcggcgccggcatcgccgaggcgcgcgactatctcggcgccatcttcccctcggccagcggcgccatgttcgaatgcacccccgacgagggcaaggccgccttcctgcaccgtttcgccgtggcgggcgccgccgtgcgctatcgcgccatccacgcccgcgaggtgcaggacatcgtggcgctggacatcgcgctgcgccgcaacgaccgcgactgggtcgagcgcctgccgccggacctggacgcgaaactcgagaaaaagctctattacggccatttcttctgccacgtcttccatcaggactacgtggtgaaaaagggccaggactgcctggccgtcgagcatgagatgtggcgcctcttggaccggcgcggcgccgaatacccggccgagcacaatgtcggccacctttaccacgccaagccggaactggccggcttctaccggcagctggacccgaccaacagcctgaaccccggcatcggccagacctcgaaatgcgcgcattggcactag

SEQ ID NO: 5 - protein, 381 aa, Paracoccus denitrificans
MTFRANWSYPTTIKFGPGRVTELAEHCRAVGIARPLLVTDKALASLPITAQALDVLDASGLGRAVFSEVDPNPHEGNMEAGIAAYKAGGHDGVICFGGGSALDLGKMIALMADQTVSVWDLEDIGDWWTRADAGKIAPIIAVPTTAGTGSEVGRAGVLTNSATHKKKIIFHPRLMPAVTICDPELTVGMPKFITAGTGMDAFAHCLEAFCSPHYHPMSQGIALEGLRLVNEYLPRAYATPDDLEARAHMMSAAAMGAVAFQKGLGAIHSLSHPVGAVYGTHHGTTNAVVMPMVLDFNRSAIEDRLARAADYLGIKGGFDGFRARVIQLRSELAIPQNLTRLGVQTERLDELTEMALEDPSCGGNPVEMTRENTRALFESCM

SEQ ID NO: 6 - DNA, 1146 bp, Paracoccus denitrificans
  source 1..1146: mol_type = other DNA
atgacgtttcgtgcgaactggtcctatccgaccaccatcaaattcggcccgggccgcgtcaccgaactggccgagcattgcagggccgtgggcatcgcgcgtccgctgctggtgaccgacaaggcattggcgagcctgcccataaccgcgcaggccctggatgttctggatgcctccggccttggccgcgcggtcttttccgaggtcgatccgaacccgcacgaaggcaatatggaggccggcatcgccgcctacaaagccggcggacatgacggcgtgatctgctttggcggcggctcggcgctggatctgggcaagatgatcgcgctgatggcggaccagaccgtatccgtttgggacttggaggacatcggcgactggtggacccgcgcggatgccggcaagatcgcgcccatcattgccgtgccgacgaccgcgggcacgggctccgaggtcgggcgcgccggggtcctgacgaactcggccacgcacaagaagaagatcatcttccaccccaggctgatgcctgccgtcacgatctgcgaccccgaactgaccgtgggcatgccgaaattcatcaccgccggcaccggcatggatgccttcgcccattgcctcgaagcgttctgctcgccccattaccaccccatgtcgcagggcatcgcgcttgaggggctgcggctggtcaacgaatacctgccccgcgcctatgccacgcccgacgacctcgaggcccgcgcgcacatgatgagcgccgccgccatgggggccgtcgccttccagaaggggctgggcgcgatccacagcctgagccatccggtcggcgcggtctacggcacccatcacggcaccacgaatgcggtggtcatgccgatggtgctggacttcaaccgatccgccatcgaggaccggcttgcccgcgccgccgactatcttggcatcaagggcggcttcgacggcttccgcgcacgggtgatccagctgcgcagcgagcttgccatcccccagaacctcacccggctgggggtccagaccgaacgtctggacgaattgaccgagatggcgctggaggacccgagctgcggcggcaacccggtcgagatgacgcgcgagaacacccgcgcgctgtttgaaagctgcatgtga

SEQ ID NO: 7 - protein, 462 aa, Paracoccus denitrificans
MSKTIALISPATGRTLVERQTLGIEDARAAVARARAAQPEWAALPLDERIARIRAGIEALNAMKDAIVPELADQMGRPIRYGGEFGGVNERAGHMMKIAAQALAPTVVEDSDHFAREIRREPVGVVFVIAPWNYPFLTAVNTVVPALVAGNAVILKHASQTLLAGERLAEALHRGGVPAEVMQNVVLDHQTTEALIAGRSFGFVNFTGSVAGGRAIERAAAGTFTATGLELGGKDPGYVRADADLDAAVDGLMDGAMFNSGQCCCGIERIYVHESLFDAFVAKAVDWVNAQKLGNPRDPDTTMGPMAHRRFADLVRAQVSEAVAQGARPLIDPANFPADDGGAYLAPQVLVDVTHDMRVMREESFGPVVGIMPVRDDAEAIGLMNDCDYGLTASIWTADADAAARIGSRLETGTVYMNRCDYLDPALCWTGCKDTGRGAALSGLGYLAVTRPKSYHLKKVTK

SEQ ID NO: 8 - DNA, 1389 bp, Paracoccus denitrificans
  source 1..1389: mol_type = other DNA
atgagcaagaccatcgccctgatatcgcccgccaccggcaggacccttgtcgaaaggcagaccctgggcatcgaggacgcgcgcgccgccgttgcccgcgcccgtgccgcccagcccgaatgggcggcgctgccgctggacgagcggatcgcccgcatccgcgccgggatcgaggcgctgaacgccatgaaggatgcgatagtgcccgaactggccgatcagatgggccgcccgatccgttacggcggcgaattcggcggcgtcaacgaacgcgccggccatatgatgaagatcgcggcccaggctcttgccccgaccgtggtcgaagacagcgatcatttcgcgcgcgagatccggcgcgagccggtgggcgtggtctttgtcatcgcgccctggaactatcccttcctgaccgccgtcaacacggtcgtgcccgccctggtcgccggcaatgccgtgatcctgaaacatgcgagccagaccctgctggcgggcgagcgtctggccgaggcgctgcatcggggcggcgttcccgccgaggtgatgcagaatgtcgtcctggaccaccagacgaccgaggcgctgatcgccgggcgcagcttcggcttcgtgaacttcacgggctcggtcgccggagggcgtgcgatcgagcgggccgccgcggggacctttaccgcgaccgggctggagctgggcggcaaggatcccggctatgtccgcgccgatgccgatctggatgcggcggtggatgggctgatggacggcgccatgttcaacagcggccagtgctgctgcgggatcgagcggatctatgtccacgaaagcctctttgacgccttcgtggccaaggccgtcgattgggtgaacgcgcagaagctgggcaatccccgcgacccggatacgacgatggggccgatggcgcaccggcgctttgccgatctggtccgcgcgcaggtctccgaggccgtggcgcagggggcaaggccgctgatcgacccggcgaactttcccgccgatgatggcggcgcctatcttgcgccgcaggtgctggtcgacgtgacccacgacatgcgcgtgatgcgagaggaaagctttggccccgtcgtcggcatcatgccggttcgcgacgatgccgaggcgatcgggctgatgaacgattgcgactatgggctgaccgcctcgatctggaccgccgatgccgatgccgccgcccggatcggcagccggcttgaaaccggcaccgtctacatgaaccgctgcgactatctggacccggcgctgtgctggaccggctgcaaggacaccggacgcggcgcggcgttgtcgggccttggctatctggccgtgacgcggccgaaatcctaccatctgaaaaaggtgacgaaatga

SEQ ID NO: 9 - protein, 336 aa, Paracoccus denitrificans
MPAVHATDPTRSRLKVTVTRRLPEAVETRMSELFDVSLNAEDRRMSREELVAAMRVSDVLVPTITDHIDAAMLAQAGDRLKLIANYGAGVDHVDVHSARQRGILVSNTPGVVTEDTADVVMALILGVTRRLPEGMAEMQAGRWQGWSPTAHLGGRLGGRRLGILGMGRIGQAVARRANAFGMQVHYHNRRRLRPEVEAELQATYWESLDQMLARMDIVSVNAPHTPSTFHLLNARRLKLLKPSAVVINTSRGEVIDENALTRMLRAGEIAGAGLDVFEHGHEINPRLRELPNVVLLPHMGSATIEGRVEMGEKVIINIKTFADGHRPPDLVVPSML

SEQ ID NO: 10 - DNA, 1011 bp, Paracoccus denitrificans
  source 1..1011: mol_type = other DNA
atgccagccgttcatgcgaccgatccgacccgctcgcgccttaaagttaccgtgacccggcgcctgcccgaggcggtcgagacccggatgtccgagcttttcgacgtttccctgaacgccgaagaccgcaggatgagtcgcgaggaactggtcgccgccatgcgcgtctcggatgtgctggtgccgacgatcaccgatcatatcgacgccgccatgctggcccaggccggcgaccggctgaagctgatcgccaattacggcgcgggggtggaccatgtcgacgtgcattccgcccgccagcgcggcatcctggtcagcaacaccccgggcgtcgtgaccgaggacacggccgatgtggtcatggcgctgatcctgggcgttacccggcgcctgcccgagggcatggccgagatgcaggccggccgctggcagggctggtcgcccaccgcgcatctgggcgggcggctgggcgggcggcgcctgggcatcctgggcatgggccgcatcgggcaggcggtggcgcggcgggcgaacgccttcggcatgcaggtgcattatcacaaccgccgccgcctgcgccccgaggtcgaggccgagttgcaggcgacctactgggaaagcctggaccagatgctggcgcgcatggatatcgtcagcgtgaacgcgccgcatacgccctcgaccttccatctgctgaatgcgcggcggttgaagctgctgaagccctcggccgtggtgatcaacacctcgcgcggcgaggtcatcgacgagaacgcgctgacccggatgctgcgcgcgggcgagatcgcgggtgccggcctggacgttttcgagcatgggcacgagatcaacccccgcctgcgcgagctgcccaatgtcgtgttgctgccgcatatgggttcggccaccatcgaggggcgggtcgagatgggcgagaaagtcatcatcaacatcaagaccttcgccgacggccatcggccgccggacctggtggtgccctcgatgctgtga

SEQ ID NO: 11 - protein, 640 aa, Paracoccus denitrificans
MTPKGRTGGKGMARFASVADRDAVEAEMPYAERQVPHTVYQALTETRDRHPQRPAISFQLFSDPKAPARTLTWTELHERVTETANLFRSLGVGPDDVVAYLLPNCIEAPVVLLAGATAGIVNPINPLLEPDHIAAILRETGAKVLVTLKSFPKSEVAQKAADAVAQAPNVQTVLEVDLRGYLTGVKRLLVPLMRPKVTARHHAKVMDFDAAASAQKHNRLTFDEPAEDRVAAFFHTGGTTGMPKVAQHKQSGMIYNGWLGGTLLFTETDVLMCPLPMFHVFAAYPVLMSCLMSGAQLVMPTPAGYRGEGVFDNFWKLIERWQATFLITVPTAIAALMQRPVNADVSSLKTAISGSAPLPIELYNRFKAATGVEIAEGYGLTEATCLVSCNPINGLKKVGSVGIPLPHTHVRILQRRNGGFHECATDEIGEICVANPGVFEGSTYTEADKNHDLFAESRFLRTGDLGRMDADGYLWITGRAKDLIIRGGHNIDPAEIEDALLSHPKVAAVAAIGQPDSFAGELPCAYVELIAGAEVGLDELMEHARTHIHERAAVPKHVEILPELPKTTVGKIFKPDLRKLAIRRVYDSALAEAGLAAEVGEVVDDRKRGLVAHIRPKGQVDRSAVEQLLGQYALPWEWVG

SEQ ID NO: 12 - DNA, 1923 bp, Paracoccus denitrificans
  source 1..1923: mol_type = other DNA
atgaccccgaaagggagaacgggaggtaaaggaatggcaaggttcgcaagcgtcgcagaccgcgatgcggtcgaggcggagatgccctatgcggagcgccaggtgccgcatacggtctatcaggccctgaccgagacccgtgaccgccatccccagcgccccgcaatcagcttccagctgttctcggaccccaaggctccggcccgcacgctgacctggaccgagctgcacgagcgggtgaccgagacggcgaacctgttccgcagcctgggcgtcgggccggacgatgtcgtcgcctatctgctgcccaactgcatcgaggcgccggtcgtgctgctggcgggcgcgacggcggggatcgtgaacccgatcaacccgttgctggagcccgaccacatcgccgccattctgcgcgagaccggcgccaaggtgctggtgacgctgaaaagctttcctaaatccgaggtcgcccagaaggcggccgatgcggtggcccaggcgccgaacgtgcagaccgtgctggaggtcgacctgcgcggctacctgaccggggtgaaacgcctgctggtgccgctgatgcgacccaaggttaccgcgcggcatcacgccaaggtcatggatttcgacgccgccgccagcgcgcagaagcacaaccgcctgaccttcgacgaaccggccgaggatcgcgtcgccgccttcttccacaccgggggcaccaccggcatgcccaaggtcgcccagcacaagcagtcgggcatgatctataacggctggctgggcggaacgctgctgttcaccgaaaccgacgtgctgatgtgcccgctgccgatgtttcacgtcttcgcggcctatccggttctgatgtcctgcctgatgtcgggcgcgcaactggtcatgccgacgcccgcgggatatcgcggcgaaggcgtgttcgacaacttctggaagctgatcgagcgctggcaggcgaccttcctgatcaccgtgcccaccgccatcgccgcgctgatgcagcgccccgtgaatgccgatgtctcttcgctcaagaccgcaatctcgggttctgcgcccttgccgatcgagctttacaaccgcttcaaggcggcgaccggggtcgagatcgccgagggctacgggctgaccgaggcgacctgcctcgtgtcctgcaacccgatcaacgggctcaagaaggtcggctcggtcggcatccccctgccccatacccatgtgcgcatcctgcaacgccggaacggcggtttccacgaatgcgccaccgacgagatcggcgagatctgcgtggccaatcccggcgtcttcgagggctcgacctataccgaggccgacaagaaccacgatctcttcgccgaaagccgcttcctgcgcaccggagacctcggccggatggatgccgacggctacctgtggatcaccgggcgggccaaggacctgatcatccgcggcggccacaacatcgaccccgccgagatcgaggatgcgctgctgtcccatcccaaggtcgccgcggtggccgccatcggccaacccgattcctttgccggcgagctgccctgcgcctatgtcgagctgatcgcgggcgccgaggtcgggctggacgagttgatggagcatgcccgcacccatatccacgaacgcgccgcagtgccgaagcatgtcgagattctgcccgaactgcccaagaccaccgtcggcaagatcttcaagcccgacctgcgcaagctggcgatccgccgcgtctatgactccgccctggccgaggccgggctggcggccgaggtgggcgaggtggtcgatgaccgcaagcgcggcctggtcgcccacatccgtcccaagggccaggtcgaccgcagcgccgtcgaacagctgctgggccaatacgccttgccgtgggagtgggtcggctag

SEQ ID NO: 13 - protein, 386 aa, Paracoccus denitrificans
MSLDPETLAQFLETLDRFVRERLIPNEERVADGDAIPPELVQEIREMGLFGMSIPEEHGGIGLTMAEEVQAALVLGQASPVFRSLVGTNNGIGSQGIIIDGTPEQKAHYLPQLASGEMIASFALTEPDAGSDAGSLRCSARLDGDHYVLNGTKRFITNAPHAGLFTVFARTDPDSKSAAGVTAFLVEAGTPGLHLGPRDRKMGQKGSHTCDVILEDCRVPASAIIGGPDRLGQGFKTAMKVLDRGRLHISAVCVGAAERLIRDSLAYAMERRQFGEPIAEKQLVQAMLADSRAEAYAARCMIEETARRKDAGLSVSTEAACCKMYASEMVGRVADRAVQIHGGAGYMAEYAVERFYRDVRLFRIYEGTTQIQQLVIARNMIREASG

SEQ ID NO: 14 - DNA, 1161 bp, Paracoccus denitrificans
  source 1..1161: mol_type = other DNA
atgagcctcgaccccgaaacgcttgcgcaattcctcgagaccctggaccgcttcgtgcgcgaacgcctgatcccgaacgaagagcgggtggccgatggcgacgcgatcccgcccgaactggtgcaggagatccgcgagatgggcctgttcggcatgtcgatccccgaggaacatggcggcatcggcctgaccatggccgaagaggtgcaggccgcgctggtcctgggccaggcctcaccggtgttccgctcgctggtgggcacgaataacggcatcggctctcaggggatcatcatcgacggcacgcccgagcagaaggcgcattacctgccacagctggcctcgggcgagatgatcgcctctttcgcgctgaccgagcctgatgccgggtcggacgcgggctcgctgcgctgttcggcgcggctggacggcgatcactatgtcctgaacgggaccaagcggttcatcaccaacgcgccccatgccgggctgttcaccgtcttcgcccgcaccgatcccgacagcaagtcggccgccggcgtcaccgccttcctggtcgaggccggcacccccggcctgcatctggggcccagggaccgcaagatgggccagaagggatcgcatacctgcgatgtcatcctggaagattgccgcgtcccggcaagtgcgatcatcggcggccccgacaggctgggccagggcttcaagaccgcgatgaaggtgctggaccggggccggctgcacatctcggcggtctgcgtgggcgcggcggaacggctgatccgcgacagcctggcctatgcgatggagcgcaggcagttcggcgagcccatcgccgaaaagcagctggtgcaggcgatgctggccgacagccgcgccgaggcctatgccgcccgctgcatgatcgaggaaaccgcccggcgcaaggatgccggcctgagcgtctcgaccgaggcggcctgctgcaagatgtatgccagcgagatggtcggccgcgtggccgaccgcgcggtgcagatccatggaggggcgggctacatggccgaatatgcggtcgaacgcttctatcgcgacgtgcggctgttccgcatctatgaaggcacgacccagatccagcagctggtgatcgcccgcaacatgatccgcgaggccagcggctga

SEQ ID NO: 15 - protein, 268 aa, Paracoccus denitrificans
MRDISQLNLTHLLFDMDGDGIATVTLNRAAKRNALNAETIEELVAVFSALPASGARAVVLRAEGPHFCAGLDLVEHGREERSPAEFMRICLRWHEAFNKIEYGGIPVIAALKGAVVGGGLELASSVHIRVMDETTYFGLPEGQRGLFTGGGATIRVPRLIGQARMMDMMLTGRLYSGDEAVQVGLAQYRVADSEAQAYDLARRVAQNTPLSNFAVCSAISHMQNMSGLDAAYAEAMVAGIVNTQDAARGRLDSFAQGTAQKIKPGEAG

SEQ ID NO: 16 - DNA, 807 bp, Paracoccus denitrificans
  source 1..807: mol_type = other DNA
atgcgcgacatttcgcagttgaacctgacccacctgctcttcgacatggacggggacggcatcgccaccgtcaccctgaaccgcgccgccaagcgcaacgccctgaatgccgagacgatcgaggaactggtcgcggtcttttccgccctgcccgcctcgggcgcccgtgccgtggtgctgcgcgccgaggggccgcatttctgcgccgggctggacctggtcgagcacgggcgcgaggaacgcagccctgccgagttcatgcgcatctgcctgcgctggcacgaggcgttcaacaagatcgaatatggcggcattccggtcatcgccgcgctcaagggcgcggtggtgggcggcgggctggaactggcctcgtcggtccatatccgggtgatggacgagaccacctatttcggcctgcccgaggggcagcgcgggctgttcaccggcggcggcgccacgatccgcgtgccccggctgatcggccaggcgcgcatgatggacatgatgctgaccggccggctgtattccggggacgaggcggtgcaggtcgggctggcgcaataccgcgtggccgacagcgaggcgcaggcctacgaccttgcccgccgggtggcgcagaacacacccctgtcgaatttcgcggtctgctcggcgatctcgcatatgcagaacatgtcggggctggacgccgcctatgccgaggccatggtcgccggcatcgtcaacacccaggacgccgccaggggaaggctggacagctttgcccagggcacggcgcaaaagatcaagccgggcgaggcaggctga

SEQ ID NO: 17 - protein, 291 aa, Paracoccus denitrificans
MAIQSVGVIGAGQMGNGIAHVFALAGYDVLMTDISREALDKAVAQIDHNLERQVSRGKVSAEDKAAAMRRITTTMTLSDLGKTDLIIEAATERETVKQAIFEDLLPHLKPETILTSNTSSISITRLASRTDRPERFMGFHFMNPVPVMQLVELIRGIATNEETYKALVEVVEKIGKTSASAEDFPAFIVNRILMPMINEAVYTLYEGVGSVKSIDQSMKLGANHPMGPLELADFIGLDTCLAIMNVLHEGLADTKYRPCPLLVKYVEAGWLGRKTGRGFYDYSGEEPVPTR

SEQ ID NO: 18 - DNA, 876 bp, Paracoccus denitrificans
  source 1..876: mol_type = other DNA
atggcgattcaatcggtgggcgtgatcggcgccggacagatgggcaatggcatcgcgcatgtctttgccctggcgggttatgacgtgctcatgaccgacatctcgcgcgaggcgctggacaaggccgtggcgcagatcgaccacaacctggaacgccaggtcagccgcggcaaggtctcggccgaggacaaggccgcggcgatgcggcgcatcaccaccaccatgacgctttccgacctgggcaagaccgacctgatcatcgaggccgccaccgagcgcgagaccgtcaagcaggcgatcttcgaggatctgctgccgcatctgaagcccgagaccatcctgacctcgaacacctcgtcgatctcgatcacccgccttgccagccgcaccgaccggcccgagcgcttcatgggcttccacttcatgaacccggttccggtcatgcagctggtcgagctgatccgcggcatcgccaccaacgaggagacctacaaagccctggtcgaagtggtcgaaaagatcggcaagacctcggccagcgccgaggatttcccggccttcatcgtcaaccgcatcctgatgccgatgatcaacgaggcggtctatacgctttacgagggcgtcggctcggtcaagtccatcgaccagtcgatgaagctgggcgccaaccacccgatggggccgctggaactggcggatttcatcggcctcgacacctgcctggcgatcatgaacgtgctgcacgaggggctggcggacacgaaataccggccctgcccgctcttggtgaaatatgtcgaggcaggctggctgggccgcaagaccgggcgtgggttctacgactattcgggcgaggagccggtgccgacgcgatag

SEQ ID NO: 19 - protein, 399 aa, Paracoccus denitrificans
MTEVFICDYIRTPIGRFGGALSSVRADDLGAIPLRALMARHAGLDWQAVDDVIYGCANQAGEDNRNVARMSALLAGLPVEVPGTTINRLCGSGMDAVLVAARQIAAGEAELMIAGGVESMSRAPFVLPKAESAFSRHAEIHDTTIGWRFVNPAMHAAYGTDSMPQTGQNVADDYGISREAQDAMALASQQKAAAAIASGRLAAEIAPITIPQRKGEPIVVDTDEHPRATTPEALAKLRPLFPNGSVTAGNASGVNDGAAALILASEAAARKHGLTPIARVLGGATAGVPPRIMGIGPAPASQKLMDRLGLTPADFDVIELNEAFAAQGLATLRQLGIADDDPRVNPNGGAIALGHPLGMSGARITGTAALELALTGGKRSLSTMCIGVGQGIAVALERV

SEQ ID NO: 20 - DNA, 1200 bp, Paracoccus denitrificans
  source 1..1200: mol_type = other DNA
atgaccgaagtctttatctgcgactatatccgcacgcccatcggccggtttggcggcgcgctgtcctcggtgcgcgccgacgatctgggtgccatccccttgcgcgcgctgatggcacgccatgccggcctcgactggcaggcggtggacgacgtgatctatggctgcgccaaccaggccggcgaggacaaccgcaacgtcgcccgcatgtcagcgctgctggccgggctgccggtcgaggtgccaggcacgacgatcaaccggctctgcggctcgggcatggacgcggtgctggtcgccgcgcgccagatcgcggcgggcgaggccgagctgatgatcgccggcggcgtcgagtcgatgtcgcgcgcgcccttcgtgctgcccaaggccgaaagcgccttcagccgccatgccgagatccacgacaccaccatcggctggcgcttcgtgaacccggcgatgcatgcggcctatggcaccgactcgatgccgcagaccggccagaacgtcgccgacgactacggcatctcgcgcgaggcgcaggacgcgatggcgctggcctcgcagcagaaggccgcggcggccatcgccagcggccggctcgcagccgagatcgcgccgatcacgatcccgcagcgcaagggcgagcccatcgtggtcgataccgacgagcatccccgcgccaccacgcccgaggcgctggccaagctgcgaccgctgttcccgaacggctcggtcacggccggcaatgcctcgggggtgaacgacggggcggcggcgctgatcctggcctcggaggcggcggcgaggaagcatggactgacgcccatcgcccgcgtgctgggcggggcgacggcgggcgtgccgccgcgcatcatgggcatcggcccggcgccggcctcgcaaaaactgatggatcggctgggcctgaccccggccgatttcgacgtgatcgagctgaatgaggccttcgccgcccagggcctcgccacgctgcgccagctgggcatcgccgacgacgacccgcgcgtgaacccgaacggcggcgccatcgcgctggggcatccgctgggcatgtcgggcgcccggatcaccggcaccgcggcgctggaactggcgctgacgggtggcaaacgctcgctctcgaccatgtgcatcggggtgggacaaggcatcgccgtggcgctggaaagggtctga

SEQ ID NO: 21 - protein, 344 aa, Paracoccus denitrificans
MAKTMKAAVVREFGKPLTIDEVPVPEPGPGMIQVRIQASGVCHTDLHAAEGDWPVKPNPPFIPGHEGVGFVSAVGAGVKHVKEGDRVGVPWLYTACGHCRHCLGGWETLCESQLNTGYSVNGGFADYVVADPNYVGHLPKNVDFLDIAPVLCAGVTVYKGLKVTDTKPGDWVVISGIGGLGHMAVQYAKAMGMNVAAVDIDDEKLALARKLGATVTVNAATEPDPAAAIRKQTDGGAQGVLVTAVGRKAFEQAIGMVARGGTVALNGLPPGDFPLDIFGMVLNGITVRGSIVGTRLDLQESLDFAGDGKVKATVHKAKLEDINNIFGQMHKGQIEGRMVLDMAG

SEQ ID NO: 22 - DNA, 1035 bp, Paracoccus denitrificans
  source 1..1035: mol_type = other DNA
atggccaaaaccatgaaagccgccgtcgtgcgcgaattcggcaagcccctgaccatcgacgaggtgccggtccccgaacccggccccggcatgatccaggtcaggatccaggcctcgggcgtctgccataccgacctgcacgcggccgagggcgactggccggtcaagccgaacccgcccttcattcccggccatgaaggcgtgggcttcgtctcggccgtgggcgccggggtcaagcacgtcaaggagggcgaccgcgtcggcgtgccctggctctacaccgcctgcggccattgccggcattgcctgggcggctgggaaacgctgtgcgaaagccagctcaacaccggctattcggtgaacggtggctttgccgattacgtcgtggccgatccgaactatgtcggccacctgccgaagaacgtcgatttcctggacatcgccccggtgctctgcgcgggcgtcacggtctacaaggggctgaaggtcacggataccaagcccggcgactgggtggtgatctcgggcatcggcgggctggggcatatggcggtgcaatatgccaaggccatgggcatgaacgtcgccgccgtggacatcgacgacgaaaagctggcactggcccgcaagctgggcgccacggtgacggtgaacgccgcgaccgaacccgacccggcggcggcgatcaggaaacagaccgatggcggcgcacagggcgtgctggtgacggcggtcggccgcaaggccttcgagcaggccatcggcatggtcgcacgcggcggcacggtggcgctgaacggcctgcccccgggcgacttcccgctggacatcttcggcatggtgctgaacggcatcaccgtgcgcggctcgatcgtgggcacgcggctggacctgcaggaatcgctggatttcgccggcgacggcaaggtcaaggccaccgtgcacaaggcaaagctcgaagacatcaacaacatcttcggccagatgcacaaaggccagatcgaaggccgcatggtgctggacatggcgggctga

SEQ ID NO: 23 - protein, 508 aa, Paracoccus denitrificans
MPNDQTHPFRGVNALPFEERYDNFIGGEWVAPVSGRYFTNTTPITGAEIGQIARSEAGDIELALDAAHAAKEKWGATSPAERANIMLKIADRMERNLELLATAETWDNGKPIRETMAADLPLAIDHFRYFAGVLRAQEGSISQIDDDTVAYHFHEPLGVVGQIIPWNFPLLMACWKLAPAIAAGNCVVLKPAEQTPAGIMVWANLIGDLLPPGVLNIVNGFGLEAGKPLASSNRIAKIAFTGETTTGRLIMQYASENLIPVTLELGGKSPNIFFADVAREDDDFFDKALEGFTMFALNQGEVCTCPSRVLIQESIYDKFMERAVQRVQAIKQGDPRESDTMIGAQASSEQKEKILSYLDIGKKEGAEVLTGGKAADLGGELSGGYYIEPTIFRGNNKMRIFQEEIFGPVVSVTTFKDQAEALEIANDTLYGLGAGVWSRDANTCYRMGRGIKAGRVWTNCYHAYPAHAAFGGYKQSGIGRETHKMMLDHYQQTKNMLVSYSPKKLGFF

SEQ ID NO: 24 - DNA, 1527 bp, Paracoccus denitrificans
  source 1..1527: mol_type = other DNA
atgccgaacgaccagacgcatcccttccggggcgtgaacgcgctgcccttcgaggaacgctacgacaatttcatcggcggcgaatgggtggcgcccgtctcgggcaggtatttcaccaacaccaccccgatcaccggcgccgagatcgggcagatcgcgcgctcggaggccggcgacatcgagctggcgctggacgccgcccatgccgccaaggagaaatggggcgccaccagcccggccgagcgcgccaacatcatgctgaagatcgccgaccggatggagcggaacctggagctgctcgccaccgccgagacctgggacaacggcaagccgatccgcgagaccatggccgccgacctgccgctggccatcgaccatttccgctatttcgccggcgtgctgcgggcgcaggagggctcgatcagccagatcgacgacgacaccgtcgcctatcacttccacgagccgctgggcgtggtgggccagatcatcccctggaactttccgctgctgatggcctgctggaagctggcccccgcgatcgccgccggcaactgcgtggtgctgaaaccggccgagcagaccccggccggcatcatggtctgggccaacctgatcggcgacctgctgccgccgggcgtgctgaacatcgtcaacggcttcgggctggaagccggcaagccgctggcatcctcgaaccgcatcgccaagatcgccttcaccggcgagacgacgaccggccggctgatcatgcaatatgccagcgagaacctgatcccggtgacgctggaactgggcggcaagtctccgaacatcttctttgccgatgtcgcgcgcgaggatgacgatttcttcgacaaggcgctggaaggctttaccatgttcgcgctgaaccagggcgaggtctgcacctgcccgtcgcgcgtgctgatccaggaatcgatctatgacaagttcatggaacgcgcggtccagcgcgtacaggcgatcaagcagggcgatccgcgcgaaagcgacaccatgatcggcgcgcaggcgtcgagcgagcagaaggaaaagatcctcagctatctcgacatcggcaagaaggaaggcgccgaggtgctgaccggcggcaaggcggccgacctgggcggcgagctttcgggcggctactacatcgagccgacgatctttcgcggcaacaacaagatgcggatcttccaggaggaaatcttcggcccggtggtctcggtcaccaccttcaaggaccaggccgaggcgctggagatcgccaacgacacgctttacggccttggcgccggcgtgtggtcgcgcgatgccaatacctgctatcgcatgggccgcggcatcaaggccggccgggtctggaccaactgctaccacgcctatccggcccatgcggccttcggcggctacaagcagtcgggcatcgggcgcgagacgcacaagatgatgctggaccactatcagcagaccaagaacatgctggtcagctattcgcccaagaagctgggcttcttctga

SEQ ID NO: 25 - protein, 554 aa, Paracoccus denitrificans
MSATTLSRSRPGYEQAVAQFRIEDAIAGLRGDLETGLNACVECCDRHCGADRVALRCLSADEALVEYTFEDLRALSARAANLMRDKGIRPGDVVAGLLPRTVELVATVLGAWRLGAVYQPLFTAFGPKAIEHRLKTSGAKLVVTNAAQRPKLDEVEDCPLVATLRGDGPLAPGDVDFREALAMASDQFEPVMRRGEDLFMMMSTSGTTGLPKGVPVPLRALLAFGAYMRDAIDLRETDVFWNIADPGWAYGLYYALTGPLLLGQPTILYEGGFTAETTYRIIERMGVTSLAGSPTAYRLLIAAGPEAAGAVKGRLRVVSSAGEPLNPEIIRWFGEHLAVPIHDHYGQTEMGMCVNNHHGLEHPVRPGSAGLAMPGYRIVVLDDDGNELGPNQPGVLAIDMKRSPLMWFSGYLNQATPALAGGYYRTGDSVEFEPDGSISFIGRSDDVITSSGYRIGPFDVESALIEHPAVVEAAVVGVPDPERTEIVKAFVVLAKGVEGTEALREELAQHVKKRLSAHAYPRMIDFVADLPKTPSGKIQRFVLRKAEVEKLARE

SEQ ID NO: 26 - DNA, 1665 bp, Paracoccus denitrificans
  source 1..1665: mol_type = other DNA
atgtctgcgacgaccctgtcccggtcgcggcccggctatgagcaggccgtggcgcaattccgcatcgaggacgccatcgccgggctgcgcggcgacctggagaccgggctgaacgcctgcgtcgaatgctgcgaccgccattgcggcgcggaccgggtggcgctgcgctgcctgtcggcggacgaggcgctggtggaatacacattcgaggatctgcgcgcgctgtcggcgcgggcggcgaacctgatgcgcgacaagggcatccggccgggcgacgtggtggcggggctgttgccgcgcacggtcgagctggtcgcgacggtgctgggcgcctggcggctgggcgcggtctatcagccgctgttcaccgcctttggtcccaaggccatcgagcaccggctgaagaccagcggcgccaagctggtggtgacgaatgccgcgcagcggcccaagctggacgaggtcgaggactgcccgctggtcgccaccctgcgcggcgacgggccgctggcgccgggcgatgtcgatttccgcgaggcgctggccatggcctcggaccagttcgagccggtgatgcggcgcggcgaggatctgttcatgatgatgtccacctcgggcaccacagggctgcccaagggggtgccggtgccgttgcgggcgctcttggcattcggcgcctatatgcgcgacgccatcgacctgcgcgagacggacgtgttctggaacatcgccgatccgggctgggcttacgggctttactatgcgctgaccgggcccctgctgctgggccagccgacgatcctctacgaaggcggctttaccgccgagacgacctatcgcatcatcgagcgcatgggggtgacgagccttgccggctcgcctaccgcataccggctgctgatcgcggcggggccggaggcggccggggcggtcaaggggcggctgcgggtggtcagttccgccggcgagcccctgaaccccgagatcatccgctggttcggcgagcatctggcggtgccgatccacgaccattacggccagaccgagatgggcatgtgcgtgaacaaccatcacgggctggagcatccggtgcgtccgggctcggccgggctggcgatgccgggctatcgcatcgtggtgctggacgatgacggcaacgaactgggaccgaaccagccgggcgtgctggccatcgacatgaagcgttcgccgctgatgtggttttccggctatctgaaccaggcgacgccggcgctggcggggggctattaccgcaccggcgacagcgtggagttcgagccggacgggtcgatcagctttatcgggcggtcggacgacgtgatcacgtcctcgggctatcgcatcgggcccttcgacgtggaaagcgcgctgatcgagcatcctgcggtggtcgaggcggcggtggtcggcgtgccggatcccgagcggaccgagatcgtgaaagccttcgtcgtgctggccaagggggtcgaggggaccgaggcgctgcgcgaggaactggcgcagcatgtcaagaaacgcttgtcggcccatgcctatccccggatgatcgatttcgtcgccgacctgcccaagacgccgagcggcaagatccagcgctttgtcctgcgcaaggcggaagtcgaaaaactggcccgggagtaa

SEQ ID NO: 27 - protein, 260 aa, Paracoccus denitrificans
MFEKFLSGKTAVVTGSNSGIGLGIAHELARAGADLVLNSFTDMPEDHALAESLAAEHGVEVRYVQADMSKGADCRALIEKAGACDILVNNAGIQHVAPIPDFPGEKWDAIIAINLSSAFHTTAAALPLMRKAGWGRVINIASAHGLTASEYKSAYVAAKHGIVGLTKVTALETAKEPITCNAICPGYVLTPIVEKQIPDQMKTHNMSREDVIAKVMLQRQPSGQFATVEQMGGTAVFLCSPAAEQITGTTISVDGGWTAL

SEQ ID NO: 28 - DNA, 783 bp, Paracoccus denitrificans
  source 1..783: mol_type = other DNA
atgttcgagaaattcctcagcggcaagacggcggtggtgacgggctccaattcggggatcgggctcgggatcgcgcatgaactggcgcgcgcgggcgctgatctcgtgctgaacagctttaccgacatgcccgaggaccacgcccttgccgaaagccttgccgccgagcatggcgtcgaggtgcgctatgtccaggccgacatgtccaagggcgccgactgccgcgccctgatcgaaaaggccggcgcctgcgacatcctggtgaacaatgccggcatccagcatgtcgcaccgatcccggatttcccgggcgagaaatgggatgcgatcatcgccatcaacctgagttccgcctttcacaccacggcggcggcgctgcccctgatgcgcaaggcaggctgggggcgggtgatcaacatcgcctcggcgcacgggctgacggccagcgaatacaaatcggcctatgtcgcggccaagcacggcatcgtcggcctgaccaaggtgacggcgctggagaccgcgaaggagccgatcacctgcaacgccatctgccccggctatgtgctgaccccgatcgtggaaaagcagatccccgaccagatgaagacccacaacatgagccgcgaggacgtgatcgccaaggtcatgctgcagcgccagccctcggggcaattcgccacggtcgagcagatgggcggcacggcggtcttcctgtgctcgccggcggcggagcagatcaccggcacgaccatctcggtggacggggggtggacggcgctttag

SEQ ID NO: 29 - protein, 393 aa, Paracoccus denitrificans
MSENEIVILSGARTAIGTFGGSLAGVPPIQLAATVTRAAIERAGIGPERIGTVVFGHVLNTEPRDMYLSRVAMLDAGVPDTTPAMNVNRLCGSGAQAIVSATQALILGDADFAVAGGAESMSRAPYAVPAARFGAKMGDVQMLDMMVGALTCPMGTGHMGVTAENVAREHDISRQAQDEFALESQKRAAAAIAEGRFKEQIVPIEIKTRKGMVAFDTDEHPKATDLEKLAGLKAVFQKDGTVTAGNASGINDGAAALVLARADAARAAGAKPLFRVLGYAVAGVRPEVMGIGPVPAVEALLKSTGLKIGEFDVIESNEAFAAQALAVNKGLGLDPAKVNPNGGAIALGHPVGATGALVTVKAMYELMRTGGSKGLITMCIGGGQGIALAIERI

SEQ ID NO: 30 - DNA, 1182 bp, Paracoccus denitrificans
  source 1..1182: mol_type = other DNA
atgtcagagaacgaaatcgtcatcctgtcgggggcgcgcacggccatcggcaccttcggcggcagcctcgcgggggtgcctccgatccagcttgccgcgacggtcacccgtgcggcgatcgagcgcgcggggatcggccccgagcgcatcggcaccgtggtcttcggccatgtgctgaacaccgagccgcgcgacatgtatctgtcgcgcgtggcgatgctggacgccggcgtgcccgataccacgccggcgatgaacgtgaacaggctctgcggttcgggggcgcaggccatcgtctcggccacgcaggcgctgatactgggggatgccgactttgccgtggcgggcggtgcggaatcgatgagccgcgcgccctatgccgtgccggcggcccggttcggcgccaagatgggcgacgtgcagatgctcgacatgatggtcggcgcgctgacctgcccgatgggcaccggccatatgggcgtgacggccgagaatgttgcgcgcgaacatgacatctcacgccaggcgcaggacgaattcgcgctggaaagccagaagcgcgctgcggccgccatcgccgagggccgcttcaaggagcagatcgttcccatcgagatcaagacccgcaagggcatggtggccttcgataccgacgagcacccgaaagcgaccgatctggagaagctggcgggactgaaggcggttttccagaaggatggcacggtcaccgccggcaacgcctcgggcatcaacgacggcgcggcggctctggtgctggcgcgcgcggacgcggccagggcggcgggcgccaagccgctgttccgggtgctcggctatgccgtggcgggcgtgcgtcccgaggtcatgggcatcggcccggtcccggcggtcgaggcgttgctcaagagcaccggcctgaagatcggcgagttcgacgtgatcgagtcgaacgaggctttcgccgcgcaggctttggcggtgaacaaggggctcggcctcgatccggcgaaggtgaacccgaacggcggcgccatcgccttgggccatccggtcggcgcgaccggcgccctcgtcacggtcaaggcgatgtatgaattgatgcggaccggcggcagcaaggggctgatcaccatgtgcatcggcggcggccagggcatcgcgctggctatcgagcgcatctga

SEQ ID NO: 31 - protein, 600 aa, Paracoccus denitrificans
MKNLMNGACLALLMSGTAALANDSVLAEIAKPQQWAIQMGDYANTRYSTLDQINKDNVKDLRVAWTFSTGVLRGHEGSPLVIGDVMYVHTPFPNRVFALDLNDNGKILWRYEPQQDPNVIAVMCCDTVYRGLSYADGMILLGQADTTVVALDATSGEVKWSTKIGDPGIGETLTATVVPVKDKVLVGISGGEYGVRGRMTALNLTDGSEAWKAWSTGPDEELLVDPETTTHLGKPIGADSSLNSWEGDQWQIGGGTIWGWFSYDPDLNLVYYGTGNPSTWNPSQRPGDNKWSMTIMARDADTGMAKWFYQMTPHDEWDYDGVNEMILTNQTVDGQERKLLTHFDRNGLAYTMDRETGELLVAEKYDPVVNWTTGVDMDPNSETYGRPAVVAEYSTAQNGEDENTTGVCPAALGTKDQQPAAFSPKTNLFYVPTNHVCMDYEPFRVAYTAGQPYVGATLSMYPAPNSHGGMGNFIAWDNTTGEIKWSVPEQFSVWSGALATAGDVVFYGTLEGYLKAVDAQTGEELYKFKTPSGIIGNVMTYEHGGKQYVGILSGVGGWAGIGLAAGLTNPNDGLGAVGGYASLSQYTELGGQLTVFELPG

SEQ ID NO: 32 - DNA, 1803 bp, Paracoccus denitrificans
  source 1..1803: mol_type = other DNA
atgaaaaacctaatgaatggcgcctgccttgcgctgctcatgtccggcactgcggcgctggccaatgacagcgtgctggccgagatcgccaagccccagcaatgggcgatccagatgggcgattacgcgaacacgcgatattcgaccctggaccagatcaacaaggacaacgtcaaggatctgcgcgtcgcctggaccttctcgaccggggtgctgcgcggacacgaaggctcgccgctggtgatcggcgatgtcatgtatgtccacaccccctttccgaaccgggtgtttgcgctggacctgaacgacaacggcaagatcctgtggcgctatgaaccgcagcaggatccgaacgtcattgcggtgatgtgctgcgacacggtctatcgcggcctttcctatgcggacggcatgatcctgctgggccaggccgacaccacggtcgtggccctggacgccacgagcggcgaggtgaaatggtcgaccaagatcggcgacccgggcatcggcgagacgctgaccgccaccgtcgttccggtcaaggacaaggtcctggtcggcatctcgggcggcgaatacggcgtgcgcggccggatgaccgcgctgaacctcaccgatggcagcgaggcctggaaagcctggtccaccggcccggacgaggagttgctggtcgatcccgaaacgaccacgcatctgggcaagcccatcggcgccgacagctcgctcaacagctgggaaggcgatcagtggcagatcggcggcggcacgatctggggctggttctcgtatgacccggacctgaacctggtctattacggcaccggcaacccctcgacctggaacccctcgcagcggccgggcgacaacaagtggtcgatgacgatcatggcgcgggatgccgataccggcatggccaagtggttctatcagatgacgccccatgacgaatgggactatgacggcgtcaacgagatgatcctgaccaaccagaccgtcgacgggcaagagcgcaagctgctgacccatttcgaccgcaacggcctggcctatacgatggatcgcgagaccggcgagttgctggtggccgagaaatacgatccggtggtgaactggaccaccggcgtcgacatggacccgaattcggaaacctatggccgtccggccgtggtggccgaatactcgaccgcccagaacggcgaggacgagaacaccaccggcgtctgccctgcggcactgggcaccaaggaccagcagccggcggccttctcgcccaagaccaacctgttctatgtgccgacgaaccacgtctgcatggattacgagccgttccgggtggcctataccgccggccagccctatgtcggcgccacgctgtccatgtatcccgcgccgaacagccatggcggcatgggcaacttcatcgcctgggacaacaccacgggcgagatcaagtggtccgttcccgagcagttctcggtctggtcgggcgctttggctacggcgggcgacgtggtcttctacggcacgttggagggctatctgaaggccgtcgatgcccagacgggcgaggagctttacaagttcaagaccccctcgggcatcatcggcaacgtgatgacctacgaacatggcggcaagcagtatgtcggcatcctgtcgggtgtcggcggctgggccgggatcggccttgccgcgggcctgaccaatcccaacgatgggcttggcgcggtgggcggctatgcctcgctgtcgcaatataccgagctgggcggccagctgaccgtgttcgaactgccgggctaa

SEQ ID NO: 33 - protein, 632 aa, Paracoccus denitrificans
MNRNTPKARGASSLAMAVAMGLAVLTTAPATANDQLVELAKDPANWVMTGRDYNAQNYSEMTDINKENVKQLRPAWSFSTGVLHGHEGTPLVVGDRMFIHTPFPNTTFALDLNEPGKILWQNKPKQNPTARTVACCDVVNRGLAYWPGDDQVKPLIFRTQLDGHIVAMDAETGETRWIMENSDIKVGSTLTIAPYVIKDLVLVGSSGAELGVRGYVTAYDVKSGEMRWRAFATGPDEELLLAEDFNAPNPHYGQKNLGLETWEGDAWKIGGGTNWGWYAYDPEVDLFYYGSGNPAPWNETMRPGDNKWTMAIWGREATTGEAKFAYQKTPHDEWDYAGVNVMMLSEQEDKQGQMRKLLTHPDRNGIVYTLDRTNGDLISADKMDDTVNWVKEVQLDTGLPVRDPEFGTRMDHKARDICPSAMGYHNQGHDSYDPERKVFMLGINHICMDWEPFMLPYRAGQFFVGATLTMYPGPKGDRQNALGLGQIKAYDAISGEMKWEKMERFSVWGGTMATAGGLTFYGTLDGFIKARDSDTGDLLWKFKLPSGVIGHPMTYKHDGRQYVAIMYGVGGWPGVGLVFDLADPTAGLGSVGAFKRLQEFTQMGGGVMVFSLDGESPYSDPNVGEYAPGEPT

SEQ ID NO: 34 - DNA, 1899 bp, Paracoccus denitrificans
  source 1..1899: mol_type = other DNA
atgaacaggaacaccccgaaggcaaggggcgcgtcgagccttgcgatggccgtggccatgggccttgccgtcctgacgaccgcgccggcgaccgccaatgaccagttggtcgaactggccaaggaccccgcgaactgggtcatgaccgggcgcgactacaacgcgcagaactattccgagatgaccgacatcaacaaggagaacgtcaagcagctgcggcccgcctggtcgttctcgaccggggtgctgcatgggcacgaaggtacgccgctggtcgtcggcgaccggatgttcattcacacgcccttcccgaacaccaccttcgcgctggacctgaacgagccgggcaagatcctgtggcagaacaagcccaagcagaaccccacggcccggactgtggcctgctgcgacgtggtcaaccgcggcctggcctattggccgggcgacgatcaggtcaagccgctgatcttccgcacccagctcgacggccatatcgtcgccatggatgccgagaccggcgagacgcgctggatcatggagaactcggacatcaaggtcggctcgaccctgaccatcgcgccctatgtcatcaaggacctggtgctggtcggctcctcgggcgccgagctgggcgtgcgcggctatgtcaccgcctatgacgtgaaatcgggcgagatgcgctggcgcgcctttgccaccggcccagacgaggaattgctgctggccgaggacttcaacgccccgaacccgcattacggccagaagaacctgggcctggagacctgggagggcgacgcctggaagatcggcggcggcaccaactggggctggtatgcctatgaccccgaggtggacctgttctactacggctcgggcaaccccgcgccctggaacgagaccatgcgtccgggcgacaacaagtggaccatggcgatctggggccgcgaggccaccaccggcgaggcgaaattcgcctatcagaagacgccgcatgacgaatgggactatgccggcgtcaacgtgatgatgctgtcggaacaggaggacaagcagggccagatgcgcaagcttctgacccacccggaccgcaacggcatcgtctacacgctggaccgcaccaatggcgacctgatctcggccgacaagatggacgacacggtcaactgggtgaaggaggtgcagctggataccggcctgccggtgcgcgacccggaattcggcacgcgcatggaccacaaggcccgcgacatctgtccctcggcgatgggctatcacaaccagggccatgacagctacgaccccgagcgcaaggtgttcatgctgggcatcaaccacatctgcatggattgggagcccttcatgctgccctatcgcgccggccagttcttcgtcggcgccacgctgaccatgtatccgggccccaagggcgaccgccagaacgcgctggggctgggccagatcaaggcctacgacgccatctcgggcgagatgaaatgggaaaagatggagcgcttctcggtctggggcggcaccatggccaccgcgggcgggctgaccttttacggcacgctggacggcttcatcaaggcccgcgacagcgacaccggcgacctgctgtggaagttcaagctgccctcgggcgtgatcggccatccgatgacctacaagcatgacggccggcaatatgtcgcgatcatgtatggagtcggcggctggccgggcgtgggtctggtcttcgacctggccgacccgaccgccggtctgggctcggtgggcgcgttcaagcggctgcaggagttcacccagatgggcggcggcgtgatggtcttctcgcttgacggcgagagcccctattccgacccgaacgtgggcgaatacgcgccgggtgagcccacctga

SEQ ID NO: 35 - protein, 487 aa, Paracoccus denitrificans
MTRSFDPDTLDLPRGHFIAGEHVADRGRLAMHRPSDGAAFGESPVADADMVDRAVAAGRAALAASGWGCGVPRDRTRALLKWADLIEAEAETLARFEAACSTRPVAQLPVGDIAVTAEQIRFFAEMADKEGSDLVPTRDASLGMTVDEPYGVVGAITPWNFPLSMAGWKLAPALAAGNAVVLKPSEMTPFSTLYMAELSVRAGIPAGLVNVVLGDGPVTGNAITGHPGIGKVSFTGSTGAGQAIMGNIARNGVKPMTLELGGKSPQIVFADADLDLAADCIARSITFNAGQACVAGSRVLVAAEIAEALAERLIARMADHRPGTTWDAETQYSPIISERQIARIDGIVQAAVAQGAEVLAGAARLDHPGWFYAPTLLAGVAPDSPAVTEEIFGPVLTLEPFADEEQAVAMADHPTYGLCAGIFTRDLSCALRVMRRIEAGTVWINRYGRSRDHILPTGGYKSSGIGKDLGRAAYHANRRQKSVLIDL

SEQ ID NO: 36 - DNA, 1464 bp, Paracoccus denitrificans
  source 1..1464: mol_type = other DNA
atgacccgatccttcgatcccgacactctggacctgccgcgcgggcatttcatcgccggcgaacatgtcgccgaccggggccggctggccatgcatcgcccctcggacggggcggcgttcggcgagagtccggtcgcggatgcggatatggtggatcgcgcggtcgcggccgggcgggccgcgctggccgcctcgggctggggttgcggcgtcccgcgcgaccgcacccgcgccctgctgaaatgggccgacctgatcgaggccgaggccgagacgctggcccggttcgaggccgcctgctcgacccgccccgtcgcgcaattgccggtgggcgacatcgccgtgaccgccgagcagatccgctttttcgccgagatggcggacaaggagggcagcgacctggtgccgacccgcgacgcctcgctggggatgaccgtggacgagccctatggcgtcgtgggcgcgatcacgccctggaattttccgctgtcgatggcgggatggaagctggcaccggcgctggcggcaggcaatgcggtggtgctgaagccgtccgagatgacgcccttttcgacgctttacatggccgagctttccgtgcgcgcaggcattcccgccgggctggtcaacgtggtgctgggcgacgggccggtcaccggcaatgcgatcaccggccatcccgggatcggcaaggtcagctttaccggctcgaccggggcggggcaggcgatcatgggcaatatcgcccgcaacggcgtcaagccgatgacgctggaactgggcggcaagtcgccgcagatcgtctttgccgatgccgacctggatctggccgccgattgcatcgcgcgttccatcaccttcaacgccgggcaggcctgcgtcgcgggcagccgggtgctggtcgcggcggaaatcgccgaggcgctggcggaaaggctgatcgcgcgcatggccgaccaccgtcccggcaccacatgggatgccgaaacgcaatattccccgatcatctcggaacgccagatcgcccgcatagacggcatcgtgcaggctgccgttgcccaaggggccgaggtgctggccggcgcggcgcggctggatcatccgggctggttctatgcgccgacgctgctggcgggcgtcgcccccgattcccccgccgtgaccgaagagatcttcggcccggtcctgaccttggagccttttgcggacgaagagcaggccgtcgccatggccgaccatccgacctatggcctttgcgccgggatattcacccgcgacctgtcgtgcgcgctgcgcgtcatgcgccggatcgaggccgggacggtctggatcaaccgctacggccgctcgcgcgaccatatcctgccgacggggggctacaagtcctcgggcatcggcaaggatctgggccgcgccgcctatcacgccaaccgccgccagaaaagcgtgctgatcgacctttga

SEQ ID NO: 37 - protein, 418 aa, Paracoccus denitrificans
MDFTNLGVMRAPRHLVFGAGQRGALARHAGVFGTRALIVTDTRMARDKDFLQMRQALEAQGIATQVFDGVAAELPLSCIEAGAKAGRAAGARMIIGIGGGSCLDAAKIIGLLLSHGGAPQDYYGEYKVPGPIMPLILLPTTSGTGSEVTPVAVLDDPQRAMKIGIASPHLIPEIAICDPELTLSCPPGLTAASGADAMTHAIEAFTTLRRPADSGLSLDHVFIGKNAISDSLALEAIRLIAANLARCVSHGDDLEARSAMMLGSTLAGLAFGVAGTAAAHAIQYPVGAMTHTAHGLGVATLMPYVMAWNRPSCETDFARIGAAMGLAASGDTSRQAEAAIAAIAALFAQVGIPATIAQLGVPEDRLDEIARLALSAERLIKNNPRMLDAEGMDRIVRAAHSGDLDLLTATSPRKAALQ

SEQ ID NO: 38 - DNA, 1257 bp, Paracoccus denitrificans
  source 1..1257: mol_type = other DNA
atggatttcacgaatttgggcgtcatgcgcgcgccgcggcatctggtcttcggcgcgggtcagcgcggcgcgctggcgcgccatgccggtgtatttggaacccgcgcattgatcgtcaccgatacccgcatggcgcgggacaaggattttcttcagatgcggcaggccctggaggcgcagggcatcgccacgcaggtcttcgacggcgtggccgcggaattgccgctgtcctgcatcgaggccggggccaaggccgggcgggcggccggtgcccggatgattatcggcatcggcggtggcagctgcctggatgccgccaagatcatcggcctcttgctgagccatggcggcgcgccgcaggattattatggcgaatacaaggttcccggtccgatcatgccgctgatcctgctgcccaccacctcgggaacgggatccgaggtgacgccggttgccgtgctggacgacccgcagcgggccatgaagatcggcattgccagcccgcatctgatccccgagatcgcgatctgcgacccggagctgacgctgagctgtccgccgggcctgaccgcggcctcgggcgcggacgccatgacccatgcgatcgaggcgttcacgaccctgcgccgtcccgccgattccggcctgtcgctggatcatgttttcatcggcaagaatgcgatcagcgacagcctggcgcttgaggcgatccgcctgatcgcggccaatctggcccgctgcgtcagccatggcgatgacctggaggcgcgcagcgcgatgatgcttggctcgaccctggccgggctggccttcggcgtggccggaaccgcggcggcccatgcgatccagtatccggtcggcgcgatgacgcataccgcgcatgggctgggggtcgcgaccttgatgccctatgtcatggcgtggaaccgccccagttgcgagaccgatttcgcccggatcggcgccgcgatgggcctggccgcgagtggcgacacctcccgccaggccgaggccgccatcgccgccattgccgcgctttttgcgcaggtgggcattcccgccaccatcgcccagcttggcgtccccgaggaccggctggacgaaatcgcgcggctggcgctcagcgccgaacgcctcatcaagaacaacccgcgcatgcttgacgccgaaggcatggaccggatcgtccgggccgcgcattcgggcgatcttgacctgctgaccgccacatcgccccgaaaggctgcactccaatga

SEQ ID NO: 39 - protein, 492 aa, Paracoccus denitrificans
MSYHDTISQELGFDPQALRGDLWIDGGWRKGRGGDPIAVIDPSTGNTITRIENASIDDAMDAVAAAEAALPGWAATPPRVKSEILRRCYDLMIQRKDMLARLISLENGKALPDAQGEVLYAAEFFRWFAEEAVRLNGEIYTAPSGANRIIVTHRPIGVAVMVTPWNFPAAMATRKIAPALAAGCTCVLKPATETPLTAYALAEIYAEAGVPPGVVNVLTTSRSGATVSAMLHDPRVRKLSFTGSTEVGRRLLHEAADTVISCSMELGGNAPFIVFDDADLDLAIEGAMVAKMRNGGEACTAANRFLVQKGIAPAFAERLAARMEAMTLGAGYAGETLCGPLINREALDRIAGLVSEAESHGAKTLTGGRPLDRPGFYFPPTVLTDVPPQAEITGEEIFGPVAALATFETEDEAIARANSTEYGLISYVFTSDLARGLRVSERLDSGMVGLNRGVVSDPAAPFGGTKQSGLGREGAHHGILEFCEVQYIAANW

SEQ ID NO: 40 - DNA, 1479 bp, Paracoccus denitrificans
  source 1..1479: mol_type = other DNA
atgagctatcacgacaccatctcgcaagaactcggcttcgacccgcaggcgctgcgcggcgatctgtggatcgacggtggctggcgcaaggggcgcggcggcgatccgatcgcggtcatcgacccctcgaccgggaacacgatcacgcggatcgagaatgccagcatcgacgatgccatggatgccgtcgccgccgccgaggccgccctgcctggctgggccgccaccccgccgcgcgtgaaatccgaaatcctgcgccgctgctacgatctgatgatccagcgcaaggacatgctggcccggctgataagcctggaaaacggcaaggcgctgcccgacgcccagggcgaagtgctctatgccgccgaattcttccgctggttcgccgaagaggcggtccggctgaacggcgagatctacaccgcgccctcgggtgcgaaccgcatcatcgtcacccaccgcccgatcggggtcgcggtgatggttacgccctggaacttccccgccgccatggccacccgcaagatcgcgcccgcgctggcggcgggctgcacctgcgtgctgaagcccgcgacggaaaccccgctgaccgcctatgcgctggccgagatctatgccgaggccggtgtgccgccgggcgtggtcaacgtgctgaccaccagccgttcgggcgcgacggtcagcgcgatgctgcacgatccgcgcgtccgcaagctcagcttcaccggctcgaccgaggtggggcgcaggctcttgcacgaggcggccgatacggtcatctcctgctcgatggaactgggcggcaacgcgcccttcatcgtcttcgacgacgcagatctggacctggcgatcgagggcgcgatggtcgccaagatgcgcaacggaggcgaggcctgcaccgccgccaaccgtttcctggtgcagaagggcatcgcaccggccttcgccgaacggcttgccgcgcggatggaggccatgacgctgggggcgggctatgccggggaaaccctttgcgggccgctgataaatcgtgaggcgcttgatcgcatcgcgggcctggtgagcgaggccgaaagccacggtgcgaagaccctgaccggcggtcgccccctggaccggccgggcttctatttcccgccgaccgtcctgaccgatgtgccgccccaggccgagatcaccggcgaggaaatcttcggccccgtcgcggccctggccaccttcgagaccgaggacgaggccatcgcccgcgccaattcgaccgaatacgggctgatctcctatgttttcacctcggacctggcgcgggggctgcgcgtgtcggaacggctggacagcggcatggtggggctcaatcgcggcgtggtctcggacccggcggcgccgttcggcggcaccaagcaaagcgggcttggccgggaaggcgcccatcacggcatcctcgaattctgcgaagtccagtatatcgccgccaactggtga

SEQ ID NO: 41 - protein, 490 aa, Paracoccus denitrificans
MTKHATDLKMLLKDPSLLQTRAYVAGEWVDADDGKTFPVVNPARGDVIAEVADLSRAEVARAIAAAAEAMKGWAARTAKGRAQIMRKWFDLMMENQDDLGRILTAEMGKPLPEAKGEIAYGASFIEWFGEEAKRIYGETIPGHLPDKRLTVIRQPIGVVGSITPWNFPNAMITRKCGPAIAAGCGFVGRPAAETPLSALALAVLAERAGIPKGLFSIVTSSRSSDIGKEFCENPLIRKLTFTGSTEVGRILLRQAADQVLKCSMELGGNAPFIVFDDADLDAAVEGAMASKFRNNGQTCVCANRIYVQAGVYDAFAQKLAAAVDKLRVGDGLEEGVTTGPLINQDAVEKVQEHIQDAVAGGATVVTGGKPREGLFFDPTVVTGITDKMKVATEETFGPLAPLFRFETEEEAVERANATIFGLASYFYARDIGRITRVQEALEYGIVGVNTGIISTEVAPFGGVKQSGLGREGSRHGIEDYLEMKYICLSI

SEQ ID NO: 42 - DNA, 1473 bp, Paracoccus denitrificans
  source 1..1473: mol_type = other DNA
atgaccaagcatgcgaccgacctgaagatgctgctcaaagacccctcgctcttgcagacccgcgcctatgtcgccggcgaatgggtcgatgccgacgacggcaagaccttcccggtggtgaacccggcacgcggcgacgtgatcgccgaggtggcggacctgagccgcgccgaggtcgcccgcgccatcgcggccgccgccgaggcgatgaagggctgggccgcgcgcaccgccaagggacgcgcccagatcatgcgcaaatggttcgacctgatgatggagaaccaggacgacctgggccgcatcctgaccgccgagatgggcaagccgctgcccgaggccaagggcgagatcgcctatggcgccagcttcatcgaatggttcggcgaagaggccaagcgcatctatggcgagaccatccccggccacctgcccgacaagcgcctgacggtgatccgccagccgatcggggtggtgggctcgatcacgccctggaactttcccaatgcgatgatcacccgcaaatgcggcccggccatcgccgccggctgcggcttcgtcggccgtcccgccgccgagacgccgctttcggccctggcgctggccgttctggccgagcgcgccggcatccccaaggggctgttcagcatcgtgacctcgtcgcggtcatcggacatcggcaaggaattctgcgagaacccgctgatccgcaagctgaccttcaccggctcgaccgaggtcggtcgcatcctgctgcggcaggccgccgaccaggtgctgaaatgctcgatggagctgggcggcaacgcgcccttcatcgtcttcgacgacgccgacctggacgcggcggtcgagggggccatggcctcgaaattccgcaacaacggccagacctgtgtctgcgccaaccgcatctatgtgcaagccggcgtctatgacgcattcgcgcaaaagctggccgcggcggtggacaagctgcgcgtcggcgacgggctggaagagggcgtgaccaccggcccgctgatcaaccaggatgcggtggaaaaggtccaggagcatatccaggacgccgtcgcgggcggcgccaccgtggtcaccggcggcaagccgcgcgaggggctgttcttcgacccgaccgtggtcaccggcatcaccgacaagatgaaggtggcgaccgaagagaccttcggcccgctcgcgccgctgttccggttcgagaccgaggaagaggccgtcgagcgcgccaatgcgaccatcttcggccttgcctcgtatttctatgcccgcgacatcggccgcatcacccgggtgcaggaggcgctggaatacggcatcgtgggcgtgaataccggcatcatctcgaccgaggtggcgcccttcggcggcgtcaagcaatccggcctgggccgcgagggctcgcgccacggcatcgaggattacctggagatgaaatacatctgcctatcgatctga

SEQ ID NO: 43 - protein, 482 aa, Paracoccus denitrificans
MTALKDKDLLRQQALIGGNWVDAASGAVVQVTDPATGQVMGTIPDLSAAETRAAIDAADAAFASWKKRSHAERAALLERWFDLMNQHAEDLALILTLEQGKPLSEARGEIAYGASFVKWFAEEARRIDGTVIPAPTNDRRILTLKEPVGVSAIITPWNFPNAMITRKVGPALAAGCTVVIKPSEFTPYSALALGVLAERAGIPAGVVNIVTGMPAEIGAELTANPTVRKVSFTGSTRVGSLLMAQSAPTVKRLSLELGGNAPFIVFDDADLDAAVEGAIASKFRNGGQTCVCSNRILVQAGVYDAFAEKLGAKVAAMKVGPGTQAGNDIGPMINRAALDKIARHVADAVAKGATVAARAEIPEGQYAAPVVLTGATTEMELASEETFGPVAPLFRFETEDEAVAIANGTPFGLAAYFYTENIRRAWRVAEALEFGMVGLNTGAVSTTVSPFGGVKSSGLGREGARAGIEEYLEVKAFHMGGL

SEQ ID NO: 44 - DNA, 1449 bp, Paracoccus denitrificans
  source 1..1449: mol_type = other DNA
atgacggcattgaaggacaaggatctgcttcgccagcaggcgctgatcggcggcaattgggtcgatgcggccagcggcgccgtggtccaggtcaccgaccccgcgaccgggcaggtcatgggcaccattcccgacctgtcggccgccgagacccgcgcggccatcgatgccgccgatgccgccttcgcctcgtggaaaaagcgcagccatgccgagcgcgcggcgctgctggaacgctggttcgacctgatgaaccagcatgccgaggatctggcgctgatcctgacgctggaacagggcaagccgctttccgaggcacggggggaaatcgcctatggcgcctctttcgtgaaatggttcgccgaggaggcgcggcgcatcgacggcaccgtgatcccggcgccgacgaacgaccgccgtatcctgacgctgaaggagccggtcggcgtctcggccatcatcacgccgtggaacttcccgaacgcgatgatcacccgcaaggtcggcccggccctggcggcgggctgcaccgtggtcatcaagccttccgagttcacgccttattccgcgctcgccctgggcgttctggccgagcgcgcggggattccggcgggcgtcgtcaacatcgtcaccgggatgccggccgagatcggcgcggaactgaccgccaacccgacggtgcgcaaggtcagctttaccggctcgacccgcgtcggctcgcttctgatggcgcaatccgcgccgacggtaaagcggctgtcgctggaactgggcggcaacgcgcccttcatcgtcttcgacgatgccgatctggacgcggcggtcgagggggcgatcgcgtccaagttccgcaatggcggccagacctgcgtctgctcgaaccggatcctggtgcaggcgggcgtctacgacgcctttgccgaaaagctgggcgccaaggtcgcggcgatgaaggtcggtcccggcacgcaggccggcaacgacatcggcccgatgatcaaccgcgctgcgctggacaagatcgcgcgccatgtcgcggatgccgtggcgaaaggcgcgaccgttgccgcccgggccgagatccccgaggggcaatatgccgcccccgtggtcctgacgggtgccacgaccgagatggagctggcatccgaggaaaccttcggccccgtcgcgccgcttttccgctttgagaccgaggacgaggccgtcgccatcgcgaacggcacccccttcggccttgccgcctatttctataccgagaatatccgccgtgcctggcgcgtggccgaggcgctggaattcggcatggtggggctgaacaccggcgcggtctcgaccaccgtttcgccttttggcggcgtcaagtcctcgggcctgggccgcgagggcgcgcgggcggggatcgaggaatatctggaggtgaaggccttccacatgggcgggctctga

SEQ ID NO: 45 - protein, 393 aa, Paracoccus denitrificans
MSTDPIVIVGSARTPMGGFQGDFAGVEAAALGATAIKAALGGLDPQAVDEIIMGCVLPAGQGQAPARQAALGAGLPLGAGATTVNKMCGSGMKAAMLGHDLILAGSADVVVAGGMESMSNAPYLLPKARSGYRMGHGQVMDHMFLDGLEDAYDKGRLMGTFAEDCAEAYQFTREAQDEFAISSLTRAQKAIAAGHFTGEIAPVTVRGRGGETVVDTDEQPGKARPDKIPTLRPAFRKDGTVTAANSSSISDGAAALVLMRASEAERRGLVPRARILGHATFADKPGLFPTAPIGSVRRLLERTGTAIGDYDLFEVNEAFAVVAMAAMRDLGLSHDAVNVHGGACALGHPIGASGARVLVTLLAALETHGGRRGIASLCIGGGEATAVAIERMQ

SEQ ID NO: 46 - DNA, 1182 bp, Paracoccus denitrificans
  source 1..1182: mol_type = other DNA
atgagcaccgatccgattgtcatcgtgggctcggcccgcacgccgatgggcggcttccagggcgatttcgccggggtcgaggccgccgcgctgggcgcgaccgcgatcaaggcggcgctgggcgggctggacccgcaggcggtggacgagatcatcatgggctgcgtgctgcccgccggccagggccaggccccggcgcggcaggcggcgctgggcgcgggcctgcccttgggcgccggcgccacgaccgtcaacaagatgtgcggatcgggcatgaaggcggcgatgctgggccatgacctgatcctggccggttccgccgatgtggtggtggcgggcggcatggagagcatgtcgaacgcgccctatctgctgccgaaggcgcggtcgggctatcgcatggggcatgggcaggtgatggaccacatgttcctggacgggctggaggacgcctatgacaagggccggctgatgggcacattcgccgaggattgcgccgaagcctatcagttcacgcgcgaggcgcaggacgagttcgcgatttcctcgctgacccgggcgcagaaggccatcgccgcggggcatttcacgggcgagatcgcgccggtgacggtcagggggcgcggcggcgagacggtcgtggataccgacgagcagccgggcaaggcgcggccggacaagatcccgaccttgaggccggcctttcgcaaggatggcacggtgacggcggcgaacagctcctcgatctcggacggggcggcggcgctggtgctgatgcgggcgtcggaggccgagcggcgcgggctggtgccgcgggcgcggatccttgggcacgcgacctttgccgacaagccgggcctgtttccgaccgcgcccatcggctcggtccggcggttgctggagcggacggggacggcgatcggcgattacgacctgttcgaggtcaacgaggctttcgcggtggtcgccatggcggcgatgcgcgaccttggcctgtcgcatgatgcggtgaacgtgcatggcggcgcctgcgcgctgggccatcccatcggcgcctcgggcgcgcgggtgctggtgacgctgctggcggcgctggagacgcatgggggccggcgcggcatcgcgtcgctgtgcatcggcgggggcgaggcgacggcggtggccatcgagaggatgcaatga

SEQ ID NO: 47 - protein, 690 aa, Paracoccus denitrificans
MPVHYDLAGDSAVLTFDNPPLNVLGQAMRADLARAIAQAAADRPARLILRGAGRNFVAGADAREFDGPPLDPQLNEVLDALAALPFPTIAAIHGAALGGGLEIALACRFRIAHPSATLGLPEVTLGIVPGAGGTQRLPRLVGMAAALDLLGQGRSVTAAEAESLGLIDLIADDPMAAARGVDTQTLLRALCADDRPPPAPDEAAVAAAHARADRRAPGQVAPHRAIELVATSAQEPIKAALTRERATFLDLRGSDQARALRHVFFAERAAMAQGKAWPAPAPEIARAVVVGGGNMGAAIAYALLSAGLVVRVVETDAAALDRARDNIAGLVAQGRKRGALTDAGAAELQARLSLAVGYDDLPAADLAIEAAYEDMAVKQAIFAALQDALPDSTILATNTSYLDIDLLAQGIRQPGRFLGLHFFAPAHVMKLLEIVRGEATSDQTLGAAFRLARKLGKVPVLAGVCDGFIGNRILARYRHAADILLLEGALPAQVDAAMRGFGMAMGPYEAQDMSGLDIAYANRRRQNLRDRADHRYVPIADHLVERCRRLGRKSGAGWYDYDAEGRAQPSDEVTQAILSASRDAGITRVALPAEGIAERLVLAMIAEATRILAEGIAAAPRDIDLVLVHGYGFPRWRGGLMHHADRLTPARILSRIEALAKDDPLSWSVPPLLRQLADEGRDFTSLNPSA

SEQ ID NO: 48 - DNA, 2073 bp, Paracoccus denitrificans
  source 1..2073: mol_type = other DNA
atgccggtgcattacgacctcgccggcgacagcgccgtcctgaccttcgacaatccgccgctgaacgtgctgggccaggcgatgcgcgccgatctggctcgcgccatcgcgcaggcggcggccgaccgccccgcccggctgatcctgcgcggggcggggcgcaatttcgtcgccggggcggatgcgcgcgaattcgacggcccgccgctggacccgcagctgaacgaggtgctggacgcgctggccgccctgcccttcccgaccatcgccgcgatccatggcgcggcccttggcggcgggctggagatcgcgctggcctgccgcttccgcatcgcccacccctcggccacgctcggcctgcccgaagtcaccctgggcatcgtgcccggcgcgggcggcacgcagcggttgccgcggctggtggggatggcggccgcgctggacctgttgggacagggccgcagcgtgaccgccgccgaagcggaaagcctgggcctgatcgacctgatcgccgatgacccgatggccgccgcgcggggcgtggacacgcagaccctgctccgcgccctctgcgccgacgaccgcccgccccccgcgccggacgaggcggcggtcgcggcggctcatgcgcgggccgaccggcgcgcaccgggccaggtcgcgccgcaccgggcgatcgagctggtggccacctcggcgcaagaaccgatcaaggcggcgctgacacgcgagcgcgcgaccttcctcgacttgcgcggatcggaccaggcgcgggcgctgcgccatgtgttcttcgccgaacgcgccgccatggcgcagggaaaggcctggcccgcccccgcgccagagatcgcccgcgccgtggtggtcggcggcggcaatatgggcgcggccatcgcctatgcgctgctgtcggccgggctggtggtcagggtggtcgagaccgacgccgccgcgctggaccgcgcccgcgacaatatcgccgggctggtggcgcagggccgcaagcgcggcgcgctgaccgatgccggcgccgccgagttgcaggcgcggctgtcgctggcggtgggctatgacgacctgcccgccgccgacctggccatcgaggccgcctatgaggacatggcggtcaagcaggcgatcttcgccgcgctgcaggacgcgctgcccgacagtacgatcctggccacgaatacctcgtatctggatatcgacctgctggcgcagggcatccggcagccgggccgcttcctggggctgcatttcttcgcgcccgcccatgtgatgaagctgctggagatcgtgcgcggcgaggcgacgtcggaccagaccctgggcgcggcgttccggctggcgcggaagcttggcaaggtgccggtgctggcgggcgtctgcgacgggttcatcggcaaccgcatccttgcccgctatcgccacgcggccgacatcctgctgctggaaggcgcgctgccggcccaggtcgatgccgccatgcgcggctttggcatggcgatggggccatatgaggcgcaggacatgtcggggctggacatcgcatacgccaaccgccgccgccagaacctgcgcgaccgcgctgaccaccgctacgtcccgatcgccgatcatctggtcgagcgttgcaggcgcctgggccgcaagtccggcgcggggtggtatgactatgatgccgaaggccgcgcgcagccttcggacgaggtgacgcaggcgatcctgtcggccagccgcgatgccggcatcacccgcgtggcgctgcccgccgagggcatcgccgaacggctggtcctggcgatgatcgccgaggcgacccggatcctggccgagggcatcgccgccgcgccgcgcgacatcgacctggtgctggtccatggctacggctttccgcgctggcgcggcgggctgatgcaccatgccgaccggctgacgcccgcgcgcatcctgtcccggatcgaggccctggccaaggacgacccgctgtcctggtccgtcccgccgcttctgcgccagcttgcggatgaagggcgcgatttcaccagcctgaacccatccgcctga

SEQ ID NO: 49 - protein, 773 aa, Paracoccus denitrificans
MAGKDKKPEVEAGAGDAAKPRKARGTGAKARSAAKVAKQDPAPADSEAPARGRRAKAAPAAKAAQEPVTLAKPKAAAGSGAGKPAAEPVAKSGARKPVSRARAASRSDPRTGTRRKPSAKAAAKAQAEAASLSAVDEALRPLGAVGPGAAPPMAAPAAASPTPAGTERPAAPAAEPSSAAAFAEAAFGIGSRLPEQLAQNIERIESLTQRLISALAQRRPHSPGVELPGPELFATATGAWIKLLTEQPERVLSQQVSYWGETLRHFAEAQAALARGTLKPPPSEGLRDRRFSNPLWEAHPFFNFIKRQYQINAQALEEAASALDLPEMTDRRRIEWFTRQMIDMMAPTNFLATNPDALEKALETEGESLVKGLENLVRDVEQNNGELIVSLADRDAFRVGENIGTTEGTVVARTKLYELIQYKPTTAQVHEIPLVIFPPWINKFYILDLKPQNSLIKWIVDQGYTLFVVAWKNPDPSYGDTGMDDYVTAYLEVMDRVLDLTDQKKLNVVGYCIAGTTLALTLSILKQRGDDRVNSATFFTALTDFADQGEFTAYLQEDFVSGIEEEAARTGVLGAQLMTRTFSFLRANDLVWGPAIRSYMLGEMPPAFDLLFWNGDGTNLPGRMAVEYLRGLCQQNRFVKEGFDLLGHRLHVGDVTVPLCAIACETDHIAPWRDSWRGVAQMGSKDKTFILSESGHIAGIVNPPSKKKYGHYTSDAGFDQGEQHWLDKARHHEGSWWGRWGEWLARRAGNMVEARDPGEGFGPAPGLYVHERA

SEQ ID NO: 50 - DNA, 2322 bp, Paracoccus denitrificans
  source 1..2322: mol_type = other DNA
atggcgggcaaggacaaaaaaccggaagtcgaagccggggccggcgatgcggccaaaccgcgcaaggctcgcgggactggggcaaaggcccgatccgcggccaaggtcgcgaaacaggacccggcccccgccgattcggaagcgccggcgcgcggccgccgggcgaaggctgcccccgctgccaaggccgcgcaggagcccgtgacgctggccaagcccaaggcggccgccggatccggggccggcaagcctgccgccgagccggttgccaagtccggcgcccgcaagcccgtgtcgcgtgcgcgggcggccagccgcagcgatccccgcaccggcacgcgccgcaagccctctgccaaggccgccgcgaaggcgcaggccgaagccgcatcgctgagcgcggtcgacgaggcgctgcgcccgcttggcgccgtcgggccgggtgccgcgccgcccatggccgcgcccgctgccgcgagcccgacccccgccgggaccgaaagacccgccgcccccgctgccgagccgtccagcgccgccgccttcgccgaagccgccttcggcatcggcagccgcctgcccgagcagctggcccagaacatcgaacgtatcgaatcgctgacccagcgcctgatcagcgcgctggcgcagcgccgcccacacagccccggcgtcgaactgcccggtcccgaactgttcgccaccgcgaccggcgcctggatcaagctgctgaccgagcagcccgagcgcgtgctcagccagcaggtcagctattggggcgagacgctgcgccatttcgccgaggcccaggccgcccttgcccgcggcacgctgaagccgccgcccagcgagggactccgggaccggcgcttctcgaacccgctctgggaggcgcatccgttcttcaacttcatcaagcggcaataccagatcaacgcccaggcgctggaagaggcggccagcgcgctcgacctgcccgagatgaccgaccggcgccggatcgaatggttcacccgccagatgatcgacatgatggcgccgacgaatttcctggcgaccaatcccgacgcgctggaaaaggcgctggagaccgagggcgaaagcctggtcaagggccttgagaacctggtgcgtgacgtcgagcagaacaatggcgagctgatcgtctcgctggccgaccgcgacgccttccgcgtgggcgagaacatcggcaccaccgagggcacggtggtggcgcgcaccaagctttacgagctgatccagtacaagcccaccaccgcccaggtgcacgagatcccgctggtaatctttccgccctggatcaacaagttctacatcctcgacctcaagccgcagaacagcctgatcaaatggatcgtcgatcagggctatacgctgttcgtggtggcctggaagaaccccgatcccagctatggcgataccggcatggacgattacgtcaccgcctatctggaggtgatggaccgggtgctggacctgaccgaccagaaaaagctgaacgtcgtcggctattgcatcgccgggacgacgctggcgctgacgctgtcgatcctcaagcagcgcggcgacgaccgggtgaattcggccaccttcttcactgcgctgaccgatttcgcggatcagggcgagttcaccgcctatctgcaagaggatttcgtctcgggcatcgaggaggaggcggcgcggaccggcgtgcttggcgcgcagctgatgacgcgcaccttcagcttcctgcgcgccaacgacctggtctgggggccggcgattcgcagctatatgctgggcgagatgccgcccgcgttcgacctgctgttctggaacggcgacggcaccaacctgcccgggcgcatggcggtggaatacctgcggggcctgtgccagcagaaccgcttcgtcaaggaggggttcgacctgctgggccaccgactgcatgtcggtgacgtcaccgtgccgctttgcgccatcgcctgcgagacggatcatatcgcgccctggcgcgacagctggcgcggcgtggcgcagatgggctcgaaggacaagaccttcatcctgtccgaatcgggccatatcgccggcatcgtcaatccgcccagcaagaagaaatacggccattacacctcggatgccggtttcgatcagggcgagcagcactggctggacaaggccaggcatcacgagggcagctggtggggccgctggggcgagtggctggcccgccgggcggggaacatggtcgaggcccgcgatccgggcgagggcttcggccccgcgcccgggctctacgttcacgagcgggcataa